(19) United States
(12) Patent Application Publication
(10) Pub. No.: US 2024/0309090 A1
(43) Pub. Date: Sep. 19, 2024

(54) MODULATION OF ANDROGEN SIGNALING TO INNATE IMMUNE KILLING OF PROSTATE CANCER

(71) Applicants: Brown University, Providence, RI (US); Rhode Island Hospital, Providence, RI (US)

(72) Inventors: Wafik S. El-Deiry, Providence, RI (US); Maximilian Pinho Schwermann, Providence, RI (US); Benedito A. Carneiro, Providence, RI (US)

(21) Appl. No.: 18/607,955

(22) Filed: Mar. 18, 2024

Related U.S. Application Data
(60) Provisional application No. 63/452,663, filed on Mar. 16, 2023.
(60) Provisional application No. 63/455,159, filed on Mar. 28, 2023.

(51) Int. Cl. C07K 16/28; A61K 31/4155; A61K 31/4166; A61K 31/519; A61K 39/00; A61K 45/06; A61P 35/04; A61P 37/04

(52) U.S. Cl. CPC C07K 16/2803; A61K 31/4155; A61K 31/4166; A61K 31/519; A61K 45/06; A61P 35/04; A61P 37/04; A61K 2039/505

(57) ABSTRACT

Described are improved therapies for the treatment of cancer including prostate cancer.

### CROSS REFERENCE TO RELATED APPLICATIONS

This application claims the benefit of U.S. Provisional Application No. 63/452,663 filed Mar. 16, 2023, and U.S. Provisional Application No. 63/455, 159 filed Mar. 28, 2023, which are incorporated herein by reference in their entirety.

### FIELD

The present disclosure relates to therapeutics for the treatment of cancer.

### BACKGROUND

Prostate cancer (PC) remains the most common cancer in men and is the second cause of cancer-related death in the United States after lung cancer. Although most patients present with localized disease, progression to metastatic disease and its management remains a major challenge. The androgen pathway plays a significant role in prostate cancer initiation and progression, and the blockade of this pathway is pivotal for the treatment of prostate cancer. Patients with advanced prostate cancer obtain initial benefits from androgen deprivation therapy (ADT) but, invariably, most patients progress to lethal metastatic castration-resistant prostate cancer (mCRPC) state.

mCRPC is associated with a median overall survival (OS) of 4-5 years. Standard treatments for mCRPC include AR targeted agents (e.g., enzalutamide, darolutamide, apalutamide), the CYP17 inhibitor abiraterone, chemotherapy (docetaxel, cabazitaxel), Lutetium 177-PSMA-617, and PARP inhibitors. Although the first therapeutic cancer vaccine approved more than a decade ago targeted prostate tumors (sipuleucel-T), the clinical efficacy of immunotherapy for treating PCa remains limited, with poor responses to checkpoint inhibitors as monotherapy. PCa tumors display an immunosuppressive tumor microenvironment mediated by reduced expression of human leucocyte antigen (HLA) surface expression, decreased neoantigen expression, phosphatase and tensin homolog (PTEN) protein loss, and dysfunction of interferon (IFN) type I signaling. These factors contribute to low response rates to checkpoint inhibitors in most cases of mCRPC, except for MSI-H tumors and those with CDK12 mutations. Novel immunotherapy strategies represent an active area of research to overcome immunologically cold PCa.

Emerging results suggest that AR signaling modulates the immune response by inhibiting CD8 and CD4 T cells. AR blockade enhances CD8+ T cell function and sensitizes tumors to PD-1 checkpoint inhibitors in animal models of prostate cancer. Androgen signaling in T cells suppresses IFN-γ secretion in vitro and contributes to T cell exhaustion. These effects were reversed with AR blockade and contributed to PSA and tumor responses observed among patients with mCRPC treated with pembrolizumab (anti-PD-1) and enzalutamide in a clinical trial. In addition, gene expression analysis of CD4+ T cells isolated from castrated mice identified protein tyrosine phosphatase non-receptor type 1 (Ptpn1) as a mediator of androgen-induced suppression of CD4+ T-cell differentiation. Up-regulation of interferon regulatory factor-1, 3, and 7 (IRF-1,3 and 7) and STAT4 in CD4+T cells from mice were also observed, suggesting that androgens could alter the IL-12-induced STAT4 phosphorylation and impair CD4+ T cells.

Though the role of AR signaling in CD8 and CD4 T cells is being elucidated, but the impact on NK cells has yet to be elucidated. The landscape of effective immunotherapy strategies in prostate cancer remains limited. While some metastatic cancers such as melanoma, lung cancer, and renal cell carcinoma have shown dramatic responses to immunotherapy with checkpoint inhibitors, 97% of prostate cancers generally fail to respond to these strategies. There is an unmet need for immunotherapy treatments for prostate cancer.

### SUMMARY

Provided herein are methods for treating prostate cancer. In some embodiments the methods provided herein comprise administering a combination of an androgen receptor inhibitor (ARi) and an agent that activates NK cells to a patient in need thereof.

### DESCRIPTION OF DRAWINGS

FIG. 1A shows effect of second generation androgen pathway inhibitors darolutamide and enzalutamide in prostate cancer cells-AR sensitive LNCaP-on the expression of non-classical human leukocyte antigen E (HLA-E).

FIG. 1B shows effect of second generation androgen pathway inhibitors darolutamide and enzalutamide in prostate cancer cells-AR sensitive LNCaP-on the expression of Programmed Cell Death Ligand 1 (PD-L1).

FIG. 1C shows effect of second generation androgen pathway inhibitors darolutamide and enzalutamide in prostate cancer cells-AR resistant 22Rv1-on the expression of HLA-E.

FIG. 1D shows effect of second generation androgen pathway inhibitors darolutamide and enzalutamide in prostate cancer cells-AR resistant 22Rv1-on the expression of PD-L1.

FIG. 2A shows that second generation androgen pathway inhibitors darolutamide and enzalutamide modulate the surface expression of NKG2A. Effect is reversed by DHT treatment.

FIG. 2B shows that second generation androgen pathway inhibitors modulate the surface expression of PD-1. Effect is reversed by DHT treatment. The mean fluorescence intensity (MFI) measures brightness and is a relative measure of antigen abundance.

FIG. 3A shows effect of androgen pathway inhibitors with or without (+/−) anti-NKG2A in co-culture with patient derived NK cells and PC3 cells. Graph is representative from 48 h.

FIG. 3B shows effect of androgen pathway inhibitors with or without (+/−) anti-NKG2A in co-culture with patient derived NK cells and LNCaP cells. Graph is representative from 48 h.

FIG. 3C shows representative images from GFP-transduced cell lines co cultured with patient-NK cells in a 1:1 ratio.

FIG. 4A shows tumor cells death for LNCaP cell line incubated with NK cells in a 1:1 ratio and treatment conditions quantified after 48 h. Co cultures were done in the presence of androgen pathway inhibitor (API or ARi herein) with and without TRAIL blockade (RIK-2) and IFN-γ blocking mAb.

FIG. 4B shows representative images from GFP-transduced cell lines co cultured with patient-NK cells in a 1:1 ratio.

FIG. 5A shows effect of second generation androgen pathway inhibitors darolutamide and enzalutamide on the NK mediated immune enhancement of cell death for LNCaP cell line incubated with NK-92 cells in a 1:1 ratio and treatment conditions quantified after 24 h. Experiments were also performed in the presence and absence (+/−) of IFN-γ blocking mAb (10 μg/mL).

FIG. 5B shows effect of second generation androgen pathway inhibitors on the NK mediated immune enhancement of cell death for 22Rv1 cell line incubated with NK-92 cells in a 1:1 ratio and treatment conditions quantified after 24 h. Experiments were also performed in the presence and absence (+/−) of IFN-γ blocking mAb (10 μg/mL). FIG. 5C shows representative images from GFP-transduced cell lines.

FIG. 6A shows that co cultures of PC with NK-92 cells and androgen pathway inhibitors increased NK-92 secretion of IFN-γ.

FIG. 6B shows that co cultures of PC with NK-92 cells and androgen pathway inhibitors increased NK-92 secretion of granzyme B.

FIG. 7A shows effect of androgen pathway inhibitors on NK cell infiltration in nude mice not treated with darolutamide or enzalutamide (PC3 xenograft control).

FIG. 7B shows effect of androgen pathway inhibitors on NK cell infiltration in nude mice not treated with darolutamide or enzalutamide (22Rv1 xenograft control).

FIG. 7C shows effect of androgen pathway inhibitors on NK cell infiltration in nude mice treated with darolutamide or enzalutamide for 4 weeks (PC3 xenograft).

FIG. 7D shows effect of androgen pathway inhibitors on NK cell infiltration in nude mice treated with darolutamide or enzalutamide for 4 weeks (22Rv1 xenograft).

FIG. 8A shows effect of androgen pathway inhibitors on NK cell viability in nude mice treated with darolutamide or enzolutamide for 4 weeks (PC3 xenograft).

FIG. 8B shows effect of androgen pathway inhibitors on NK cell viability in nude mice treated with darolutamide or enzalutamide for 4 weeks (22Rv1 xenograft).

FIG. 9A shows relative viability of NK-92 cells in the presence of varying concentrations of darolutamide or enzalutamine.

FIG. 9B shows relative viability of NK-92 cells in the presence of varying concentrations of darolutamide or enzalutamine as measured in a fluorimetric assay.

FIG. 10A shows that expression of NK-92 cell activation marker CD69 is increased in the presence of androgen pathway inhibitors.

FIG. 10B shows that expression of NK-92 cell activation marker CD139 is increased in the presence of androgen pathway inhibitors.

FIG. 11A shows expression of CD69 in control NK-92 cells.

FIG. 11B shows that expression of NK-92 cell activation marker CD69 is increased in the presence of enzalutamide.

FIG. 11C shows that expression of NK-92 cell activation marker CD69 is increased in the presence of darolutamide.

FIG. 11D shows expression of CD137 in control NK-92 cells.

FIG. 11E shows that expression of NK-92 cell activation marker CD137 is increased in the presence of enzalutamide.

FIG. 11F shows that expression of NK-92 cell activation marker CD137 is increased in the presence of darolutamide.

FIG. 12A shows that ARi enhances NK-cell killing of PCa cell line in vitro with the PCa cell line LNCaP and the NK-92 cell line with or without enzalutamide (10 μM).

FIG. 12B shows that ARi enhances NK-cell killing of PCa cell line in vitro with the PCa cell line PC3 and the NK-92 cell line with or without enzalutamide (10 μM).

FIG. 12C shows that ARi enhances NK-cell killing of PCa cell line in vitro with the PCa cell line DU145 and the NK-92 cell line with or without enzalutamide (10 μM).

FIG. 12D shows that ARi enhances NK-cell killing of PCa cell line in vitro with the PCa cell line 22Rv1 and the NK-92 cell line with or without enzalutamide (10 μM). Cells were cultured in a 1:1 ratio over 24 h. Tumor cells were dyed with CMAC blue dye, and the NK-92 cell line was dyed with CMFDA green dye. The ethidium homodimer red dye (1 μM) was used to assess live dead cells. Experiments were performed with tumor cells alone and in the presence of enzalutamide to account for drug-induced cell death alone. (p-value legends as follows: ns p>0.05, *p≤0.05, **P≤0.01, P≤0.001, and ****P≤0.0001).

FIG. 13A shows ARi immune enhancement killing of PCa cell lines by NK-92 cells is dependent on IFN-γ and TRAIL. Enza and daro significantly increase the secretion of cytotoxic cytokines from NK-92 cells (A1, A2). Enza and daro also reduced the secretion of immunosuppressive cytokines from the NK-92 cell line (A4 and A5). NK-92 cell line reduces que T cell chemokine CCL5 when treated with enza or daro (A6).

FIG. 13B shows that IFN-γ blockade reduced the immune enhancement killing of PCa LNCaP when treated with enza. The TRAIL blocking mAb (RIK-2) partially reduced the enza immune enhancement effect.

FIG. 13C shows that the dual blockade of TRAIL and IFN-γ reduces considerably the NK cell cytotoxic killing of PCa cell lines 22Rv1.

FIG. 13D shows that the immune activation marker CD69 is upregulated when NK-92 cells are treated with ARi (48 h).

FIG. 13E shows that the immune activation marker CD137 is upregulated when NK-92 cells are treated with ARi (48h). p-value legend for FIGS. 13A-E as follows: ns p>0.05, *p≤0.05, **P≤0.01, P≤0.001 and ****P≤0.0001).

FIG. 14A shows ARi modulates apoptotic and antiapoptotic proteins as well as pro and antitumorigenic cytokines in PCa cell lines.

FIG. 14B shows ARi modulates apoptotic and antiapoptotic proteins as well as pro and antitumorigenic cytokines in PCa cell lines. FIG. 15A shows blocking the NK cell receptor NKG2A with monalizumab potentiates the ARi-induced NK cell activation and killing of PCa cell line LNCaP. Cells were treated with enzalutamide (10 μM) and monalizumab in the presence of NK-92 cells (1:1) ratio. Monotherapy with monalizumab did not significantly enhance the NK cell killing of PCa cell line LNCaP. ARi and monalizumab significantly improved the NK cell killing of PCa cell line LNCaP.

FIG. 15B shows blocking the NK cell receptor NKG2A with monalizumab potentiates the ARi-induced NK cell activation and killing of PCa cell line 22Rv1. Cells were treated with enzalutamide (10 μM) and monalizumab in the presence of NK-92 cells (1:1) ratio. Monotherapy with monalizumab did not significantly enhance the NK cell killing of Pca cell line 22Rv1. ARi and monalizumab significantly improved the NK cell killing of Pca cell line 22Rv1.

FIG. 15C shows that when GFP-GFPexpressing PCa cells were plated with metastatic castration-sensitive prostate cancer patient-derived NK cells (1:1) ratio, enza and darolutamide enhanced the killing of the PCa cell lines over 24h. The addition of monalizumab in combination with enza and daro potentiated the killing of PCa LNCaP.

FIG. 15D shows that when GFP-GFPexpressing PCa cells were plated with metastatic castration-sensitive prostate cancer patient-derived NK cells (1:1) ratio, enza and darolutamide enhanced the killing of the PCa cell lines over 24 h. The addition of monalizumab in combination with enza and daro potentiated the killing of PCa PC3.

FIG. 15E shows Representative images of the GFP-expressing LNCaP cell line co-cultured with patient-derived NK cells and treated with ARi and monalizumab. (P-value legends for FIG. 15A-E are as follows: ns p>0.05, *p≤0.05, **p≤0.01, p≤0.001, and ****p≤0.0001).

FIG. 16A shows Prostate cancer cells' expression of HLA-E, the ligand of NKG2A, is modulated by androgen signaling. HLA-E mean fluorescence intensity (MFI) on LNCaP cells treated with enza (15 μM) and daro (20 μM) for 48 h.

FIG. 16B shows HLA-E MFI of the AR blockade-resistant 22Rv1 cell line treated with enza (15 μM) and daro (20 μM) for 48 h.

FIG. 16C shows evaluation of AR-negative cell line (PC3) and the new AR-responsive PC3-AR cell line for surface expression of HLA-E upon DHT treatment and ARi with darolutamide. The AR-negative PC3 cell line did not express significant changes in HLA-E surface expression.

FIG. 16D shows evaluation of AR-negative cell line (PC3) and the new AR-responsive PC3-AR cell line for surface expression of HLA-E upon DHT treatment and ARi with darolutamide. The AR-responsive cell line (PC3-AR) displayed increased HLA-E upon ARi.

FIG. 16E shows evaluation of AR-negative cell line (PC3) and the new AR-responsive PC3-AR cell line for surface expression of HLA-E upon DHT treatment and ARi with darolutamide. The AR-negative PC3 cell line did not express significant changes in HLA-E surface expression.

FIG. 16F shows evaluation of AR-negative cell line (PC3) and the new AR-responsive PC3-AR cell line for surface expression of HLA-E upon DHT treatment and ARi with darolutamide. The DU145 (ARresponsive) cell lines displayed increased HLA-E upon ARi.

FIG. 16G shows AR signaling dependence on the surface expression of HLA-E. The AR negative cell lines (PC3 and DU145) were transduced with AR receptor.

FIG. 16H shows AR signaling dependence on the surface expression of HLA-E. The AR negative cell lines (PC3 and DU145) were transduced with AR reporter. The m-Cherry AR transduced reporter expressed on PC3 and DU145 cell lines was functionally assessed and exhibited a GFP signal when cells were treated with increasing DHT doses.

FIG. 17A shows that androgen modulates HLA-E expression through an epigenetic mechanism. The PC3 cell lines were kept in DHT (30 nM) supplemented media for at least five days before experiments. Surface expression of HLA-E (48 h) was evaluated on the AR-responsive cell lines LNCaP and the AR-non-responsive PC3 cell line after histone deacytelase inhibitor (HDACi) treatment. HLA-E was also assessed after DHT treatment with and without HDACi. The HDACi vorinostat (0.3 μM) and panobinostat (3 nM) were used in the experiments. (P-value legends are as follows: ns p>0.05, *p≤0.05, **p≤0.01, p≤0.001, and ****p≤0.0001).

FIG. 17B shows that androgen modulates HLA-E expression through an epigenetic mechanism. The LNCaP cell lines were kept in DHT (30 nM) supplemented media for at least five days before experiments. Surface expression of HLA-E (48 h) was evaluated on the AR-responsive cell lines LNCaP and the AR-non-responsive PC3 cell line after HDACi. HLA-E was also assessed after DHT treatment with and without HDACi. The HDACi vorinostat (0.3 μM) and panobinostat (3 nM) were used in the experiments. (P-value legends are as follows: ns p>0.05, *p≤0.05, **p≤0.01, p≤0.001, and ****p≤0.0001).

FIG. 18A shows patient characteristics. Metastatic castration-sensitive prostate cancer patients (n=6) had whole PBMC's and NK cell isolation performed before and after the start of androgen deprivation therapy (ADT).

FIG. 18B shows a representative matched patient. The activation profile of NK cells was evaluated by flow cytometry. NK Cells were gated on CD56+CD45 live PBMC population.

FIG. 18C shows increased expression of Granzyme after ADT. A paired t-test was performed, and the results indicate the matched patient cohort (n=6) pre-and post-ADT.

FIG. 18D shows increased expression of Perforin after ADT. A paired t-test was performed, and the results indicate the matched patient cohort (n=6) pre-and post-ADT.

FIG. 18E shows increased expression of Granzyme and Perforin after ADT in double positive patient population. A paired t-test was performed, and the results indicate the matched patient cohort (n=6) pre-and post-ADT.

FIG. 19A shows individual cytokine analysis from PCa cell line PC3 treated with enza (10 μM) and daro (15 μM) for 24 h. The supernatant was collected for cytokine profiling.

FIG. 19B shows individual cytokine analysis from PCa cell line DU145 treated with enza (10 μM) and daro (15 μM) for 24 h. The supernatant was collected for cytokine profiling.

FIG. 19C shows individual cytokine analysis from PCa cell line 22Rv1 treated with enza (10 μM) and daro (15 μM) for 24 h. The supernatant was collected for cytokine profiling.

FIG. 19D shows individual cytokine analysis from PCa cell line LNCaP treated with enza (10 μM) and daro (15 μM) for 24 h. The supernatant was collected for cytokine profiling.

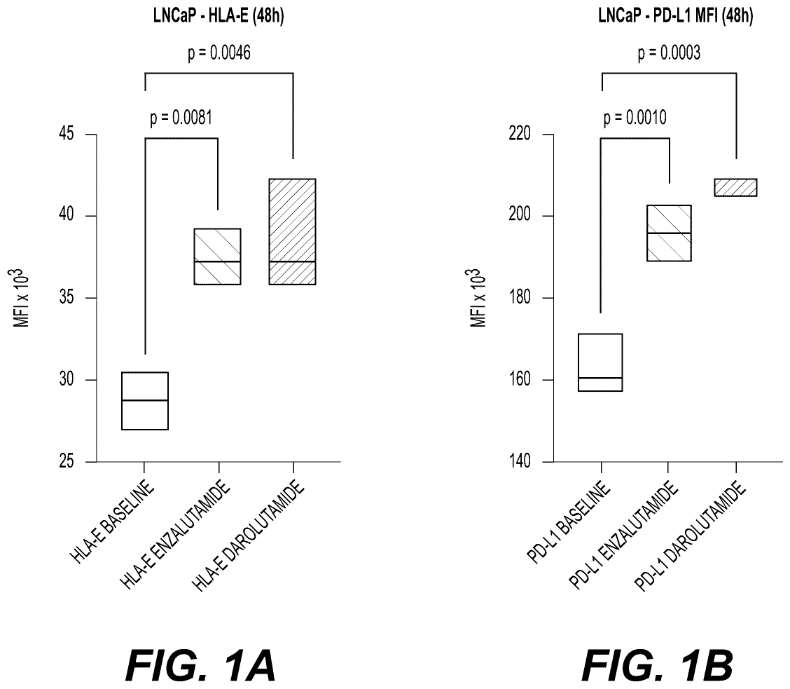

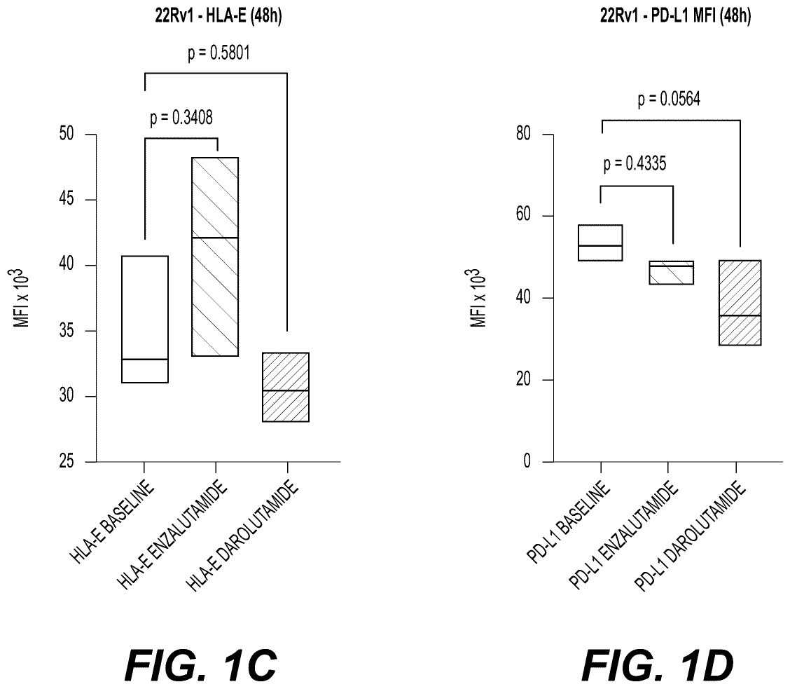

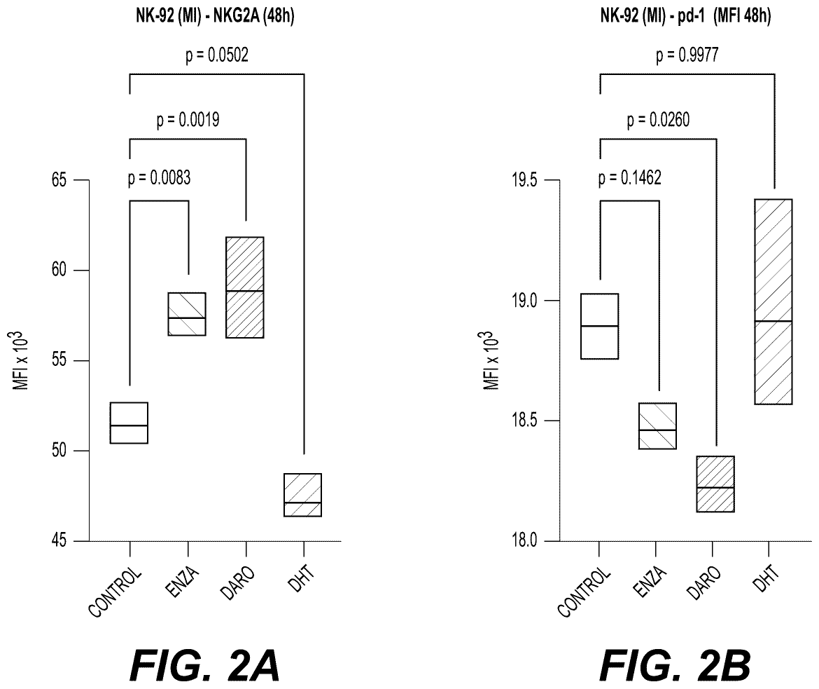

### DETAILED DESCRIPTION

Patients with metastatic prostate cancer have limited treatment options with median overall survival of approximately 5 years despite recent advances. Current standard of care treatments with androgen inhibitors, chemotherapy, and radiation have a relatively short duration of benefit because of the development of resistance. Immunotherapy with checkpoint inhibitors has limited efficacy in prostate cancer. The combination of androgen receptor inhibitors with treatments activating natural killer (NK) cells can provide an effective treatment strategy with potential to benefit patients with advanced and localized disease. NK cells are innate lymphocytes that play an important role in anti-tumour immune responses.

We investigated the effect of AR inhibitors enzalutamide (enza) and darolutamide (daro) on NK cell activation in vitro using co-culture experiments of immune cells, PCa cell lines, and patient-derived immune cells. The results revealed that AR inhibitors enhance the immune killing of prostate cancer cells by a mechanism mediated by IFN-γ and TRAIL with augmentation when combined with monalizumab, an inhibitor of the NK cell receptor NKG2A.

It has been found that androgen signaling upregulates HLA-E expression in prostate cancer cells. This could represent a novel resistance mechanism in prostate cancer cells and play a role in suppressing the immune system response to kill these cancer cells. Accordingly contemplated are approaches targeting HLA-E expression in prostate cancer cells and other tumors to develop novel therapies capable of overcoming treatment resistance.

The upregulation of HLA-E is a known immune evasion strategy in cancer cells. NKG2A, an inhibitory receptor expressed on NK cells and T cells, leads to immune evasion by binding to HLA-E expressed on cancer cells. The androgen-dependent modulation of HLA-E in PCa cells is a potential mechanism to suppress the innate immune response and protect PCa cells from NK cell killing. These results support the development of novel immunotherapy approaches in prostate cancer to promote the activation of NK cells with ARi and monalizumab, among other potential NK cell-based therapeutics.

In some embodiments, androgen receptor inhibitors include and are not limited to darolutamide and enzalutamide.

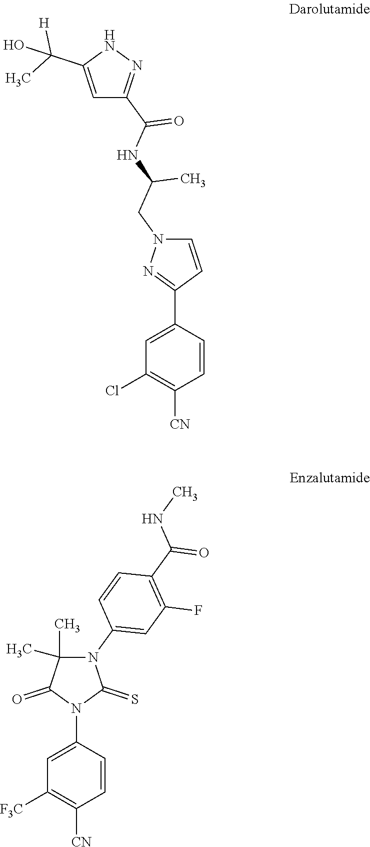 [SMILES: CC(O)c1cc(C(=O)N[C@@H](C)Cn2ccc(-c3ccc(C#N)c(Cl)c3)n2)n[nH]1 ; CNC(=O)c1ccc(N2C(=S)N(c3ccc(C#N)c(C(F)(F)F)c3)C(=O)C2(C)C)cc1F]

Other androgen receptor inhibitors contemplated within the scope of embodiments presented herein include and are not limited to flutamide, nilutamide, bicalutamide, apalutamide, proxalutamide, BMS-641988 (CAS No.: 573738-99-5) and TRC-253 (CAS No.: 2110428-64-1).

In some embodiments, NK cells may be activated by tumor necrosis factor-related apoptosis-inducing ligand (TRAIL)-inducing compounds such as ONC201.

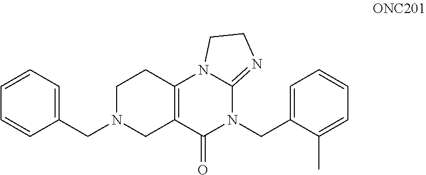 [SMILES: Cc1ccccc1CN1C(=O)C2=C(CCN(Cc3ccccc3)C2)N2CCN=C12]

In some embodiments, NK cells may be activated by NKG2A blockade (e.g., by the antibody monalizumab).

Recent findings suggest that the androgen receptor (AR) blockade sensitizes tumor-bearing hosts to effective checkpoint blockade by directly enhancing CD8+ T cell function. However, the interaction between AR signaling and the immune function of innate cells, especially NK cells, is unclear. We investigated the effect of AR inhibitors (ARi) enzalutamide (Enza) and darolutamide (Daro) on NK cell function, and the role of this blockade on immune enhancement killing of prostate cancer cell lines and their relationship to potential NK cell immune checkpoint targets.

Treatment strategies for prostate cancer based on activation of NK cells can lead to clinically relevant class of therapies for the treatment of localized and metastatic prostate cancer with the potential to increase the cure rates and outcomes of this disease. These NK-based therapies could be deployed either as monotherapies or in combination with standard of care options currently available such as surgery, radiation therapy, androgen receptor inhibitors, PARP inhibitors, checkpoint inhibitors, immunotherapy strategies, or chemotherapy.

### Definitions

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

The terms “effective amount” or “therapeutically effective amount” refer to an amount, i.e. a dosage, of therapeutic agent administered to a subject (e.g., a mammalian subject, i.e. a human subject), either as a single dose or as part of a series of doses, which is effective to produce a desired therapeutic effect (e.g., effective for influencing, reducing or inhibiting the activity of or preventing activation of a kinase, or effective at bringing about a desired in vivo effect in an animal, preferably, a human, such as reduction in intraocular pressure).

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

In one aspect, provided is a method of treating prostate cancer in a patient in need thereof comprising administering a therapeutically effective amount of an androgen receptor inhibitor and an agent that activates natural killer (NK) cells to the patient.

In some embodiments, the androgen receptor inhibitor is selected from darolutamide or enzalutamide.

In some embodiments, the androgen receptor inhibitor is selected from flutamide, nilutamide, bicalutamide, apalutamide, proxalutamide, BMS-641988 or TRC-253.

In some embodiments, the agent that activates NK cells is selected from a natural killer cell receptor NKG2A inhibitor, or a tumor necrosis factor-related apoptosis-inducing ligand (TRAIL)-inducing compound.

In some embodiments, the NKG2A inhibitor is monalizumab.

In some embodiments, the TRAIL-inducing compound is ONC201.

In some embodiments, the method comprises administration of one or more additional therapies selected from surgery, radiation therapy, poly (ADP-ribose) polymerase (PARP) inhibitors, checkpoint inhibitors, or chemotherapy.

In some embodiments, administering an androgen receptor inhibitor increases expression of non-classical human leukocyte antigen E (HLA-E) on NK cells. HLA-E inhibits NK lysis via the CD94/NKG2A receptor. In some embodiments, administering an androgen receptor inhibitor increases expression of programmed cell death ligand (PD-L1) on NK cells. PD-1 expression is rapid and transient on NK cells and occurs at a time needed to prevent excessive NK cell activation. In some embodiments, administering an androgen receptor inhibitor increases secretion of interferon gamma (IFN-γ) from NK cells. NK cell-mediated production of interferon gamma (IFN-γ) provides anti-tumor properties. In some embodiments, administering an androgen receptor inhibitor increases secretion of granzyme B from NK cells. NK cells eliminate tumor cells by releasing cytotoxic granules containing granzyme B or by engaging death receptors that initiate caspase cascades.

In some embodiments, the prostate cancer is metastatic castration-resistant prostate cancer (mCRPC).

In some embodiments, the patient has been previously treated with androgen deprivation therapy (ADT).

Further provided is a method for activating NK cells comprising contacting NK cells with an androgen receptor inhibitor.

In some embodiments, the androgen receptor inhibitor is selected from darolutamide or enzalutamide.

In some embodiments, the androgen receptor inhibitor is selected from flutamide, nilutamide, bicalutamide, apalutamide, proxalutamide, BMS-641988 or TRC-253.

Also provided herein is a method for treating any cancer associated with androgen receptor signaling in a patient in need thereof comprising administering a therapeutically effective amount of an androgen receptor inhibitor and an agent that activates natural killer (NK) cells to the patient. In some embodiments, the cancer is breast cancer. In some embodiments, the cancer is melanoma.

Further provided is a method for resensitizing treatment resistant prostate cancer cells, the method comprising modulating the expression of HLA-E in the prostate cancer cells. In some embodiments, the expression of HLA-E in prostate cancer cells is modulated by contacting the cancer cells with an androgen receptor inhibitor and monalizumab.

The results described herein support that androgen receptor inhibitors (ARi) can activate NK cells, enabling them to kill prostate cancer cells, ultimately leading to control of metastatic disease. The results show that effect of androgen receptor inhibitors on NK cells is enhanced by inhibitors of NKG2A receptor (an endogenous negative regulator of NK cell function) exemplified on these experiments by the monoclonal antibody monalizumab. These NK-activating effects of ARi are mediated by interferon-gamma (INFg) and TRAIL. Taken together, these results suggest the potential of NK cell targeting therapies in combination with ARi for treatment of prostate cancer. They also show the potential of TRAIL-inducing compounds such as ONC201 to provide additional therapeutic benefit and inform potential combinations. These findings could inform the development of combinations of treatment for patients with prostate cancer utilizing NK cell engagers or other molecules that activate NK cells in conjunction with androgen receptor blockage. Beyond prostate cancer, other tumor types could benefit from this strategy considering the importance of androgen receptor signaling in diseases such as breast cancer, and melanoma among others.

Androgen receptor inhibitors enhance NK cell killing of prostate cancer cells in vitro

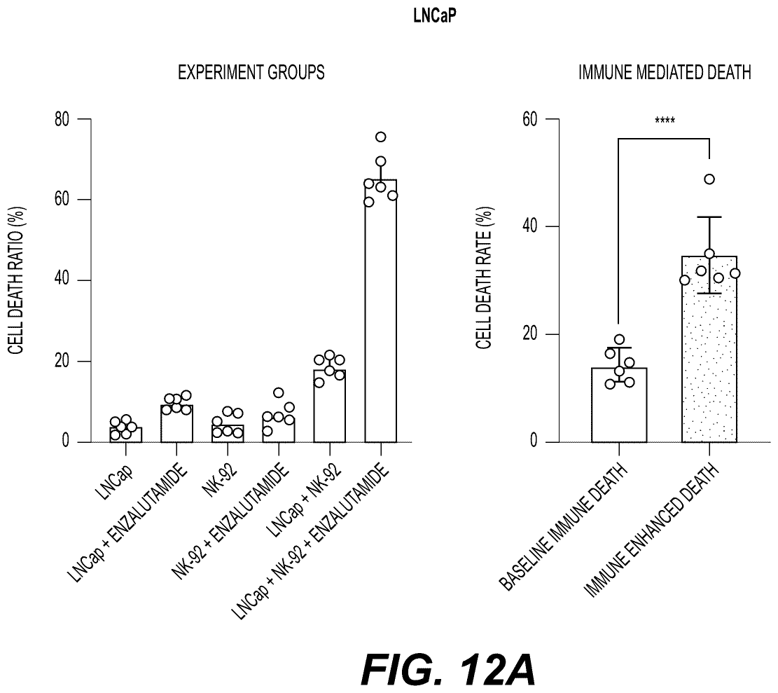

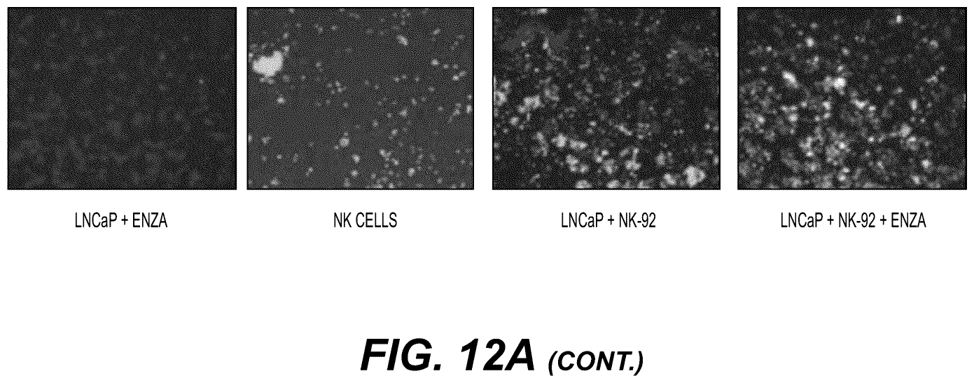

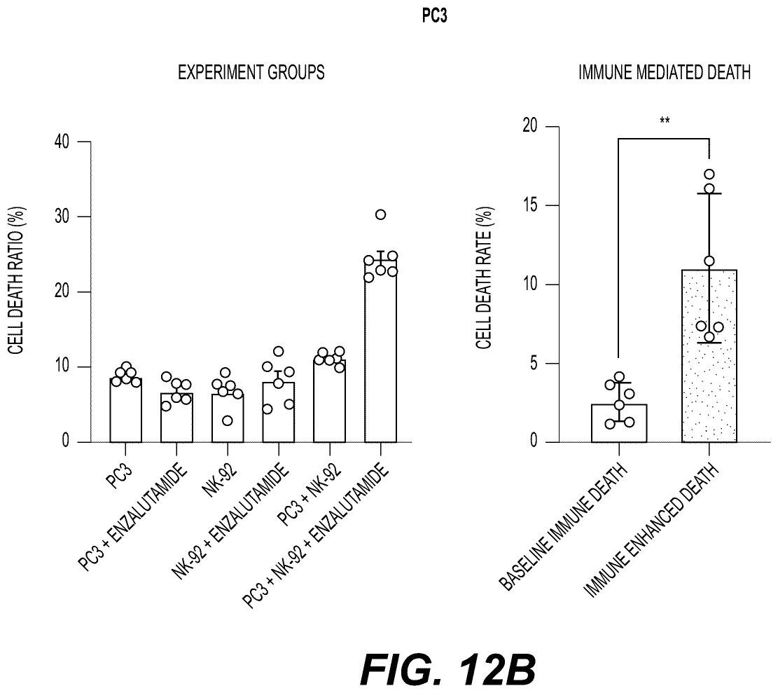

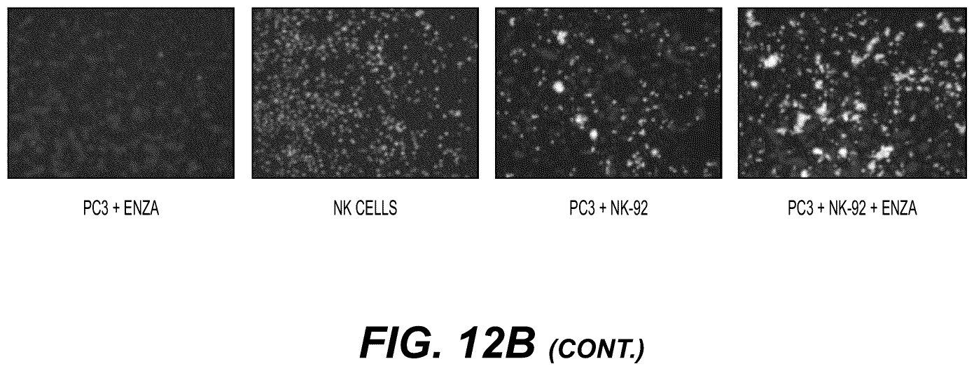

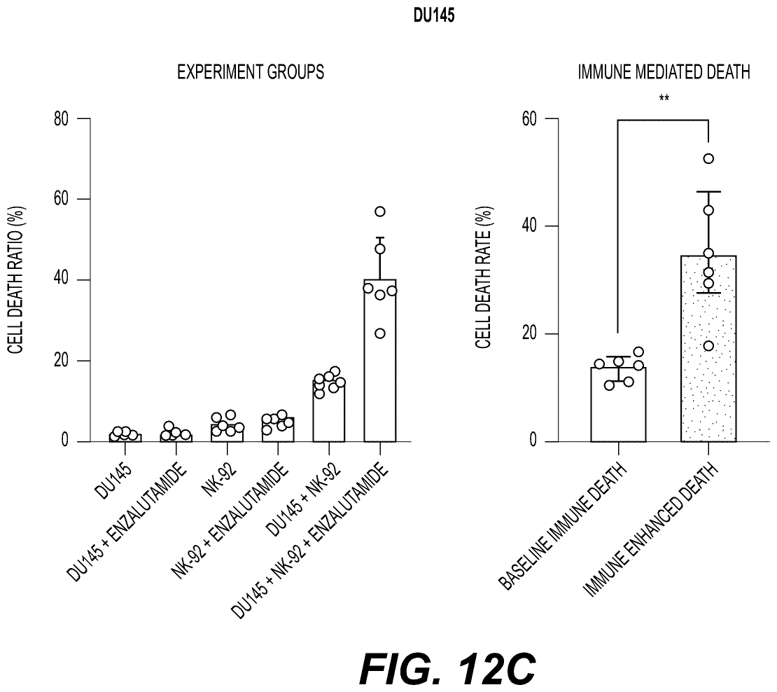

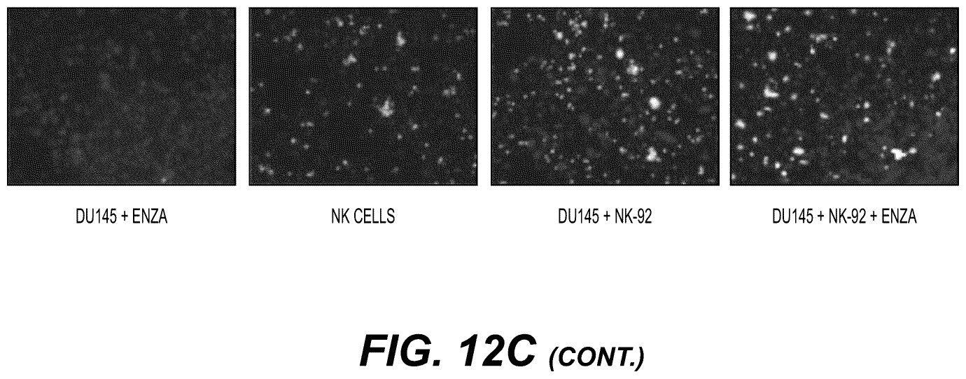

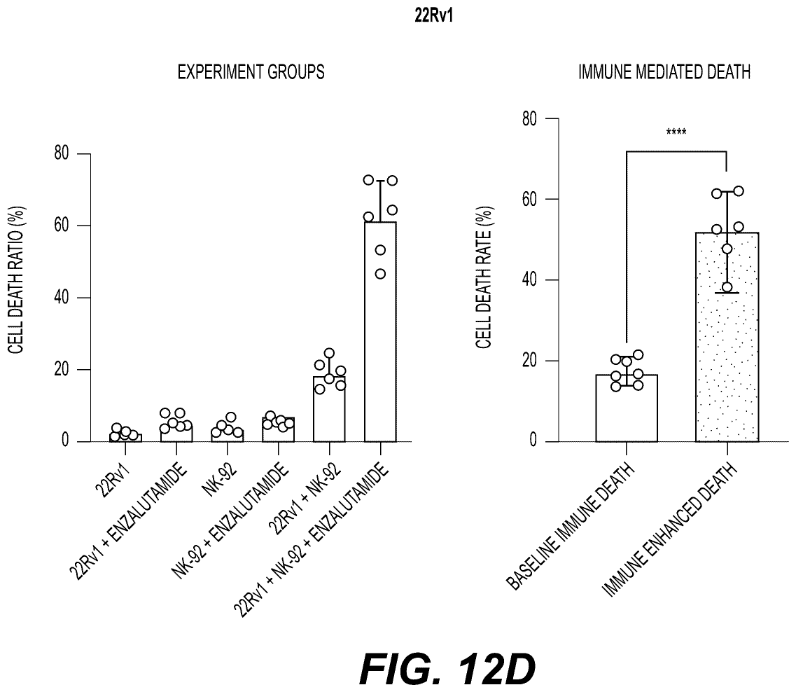

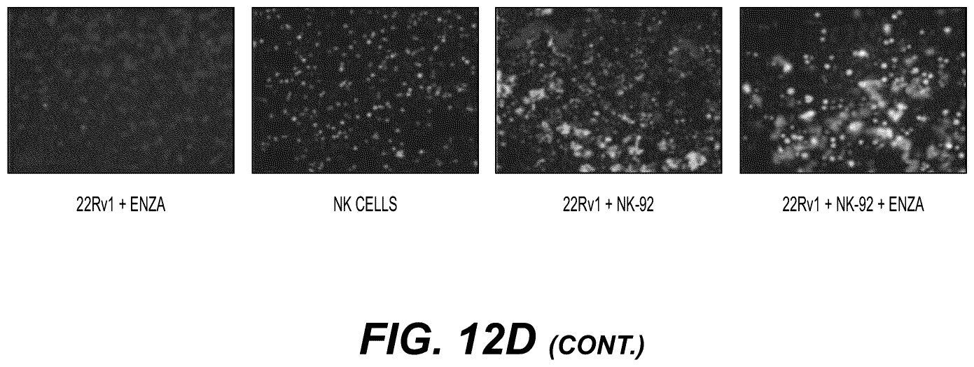

Prostate cancer cell lines (22Rv1, LNCaP, PC3, and DU145) were Ytreated with DHT (15-30 nM), and immune cells were added 24 hrs later at a 1:1 ratio. They were co-treated with enzalutamide (15 μM) or darolutamide (20 μM) for 48 h. Doses of AR inhibitors (ARi) used in the co-culture experiments were based on previous IC50 experiments. These doses did not impact NK or PCa cell viability (data not shown). Quantifications were performed using Image J. Every co-culture experiment had an NK-92 control condition alone to validate the nontoxicity of enzalutamide on the immune population (data not shown). The enzalutamide dose was noncytotoxic to the NK-92 cell line. The immune-mediated effect of NK-92 on the PCa cell lines in the presence of enzalutamide (immune enhancement death) was normalized to baseline immune and tumor cell killing in each condition [(tumor cells+immune cells)-tumor baseline death=baseline immune death]. The results display an increase in immune-mediated NK cell killing of PCa lines, irrespective of AR status or sensitivity to enzalutamide when treated with ARi. ARi treatment of co-cultures with PCa plus NK-92 cells significantly increased immune-mediated PCa killing within 24 hours independent of PCa cell line AR status and sensitivity to ARi (FIG. 12A-D). Enzalutamide significantly increased NK cell-mediated tumor killing of LNCaP in 24 hours (cell death rate: 15.6%±3.9 (control[C]) vs 48.3%±8.3 (enza); p<0.0001). This effect with enzalutamide was also observed in ARi-resistant cell lines PC3 (3.8%±1.27 (C) vs. 11.05±4.6 (enza); p<0.0017), 22Rv1 (17.07%±3.35 (C) vs 52.67±8.96 (enza); p<0.0001), and DU145 (13.45%±2.37 (C) vs 34.8±4.92 (enza); p<0.001). The enhancement of NK cell-mediated killing was also demonstrated with another ARi, darolutamide (daro;), and the maximal effect was observed within 24-30 h. Cytokine analysis was performed to investigate the activation phenotype of NK cells when treated with ARi.

The ARi immune enhancement and killing of PCa cell lines by NK cells is dependent on IFN-γ and TRAIL

The NK-92 cell line was treated with 10 nM of DHT for 72 h before treatment with enza or daro, and cytokines were measured in the culture supernatant. The secretion profile was assessed in monoculture conditions without the PCa cell lines.

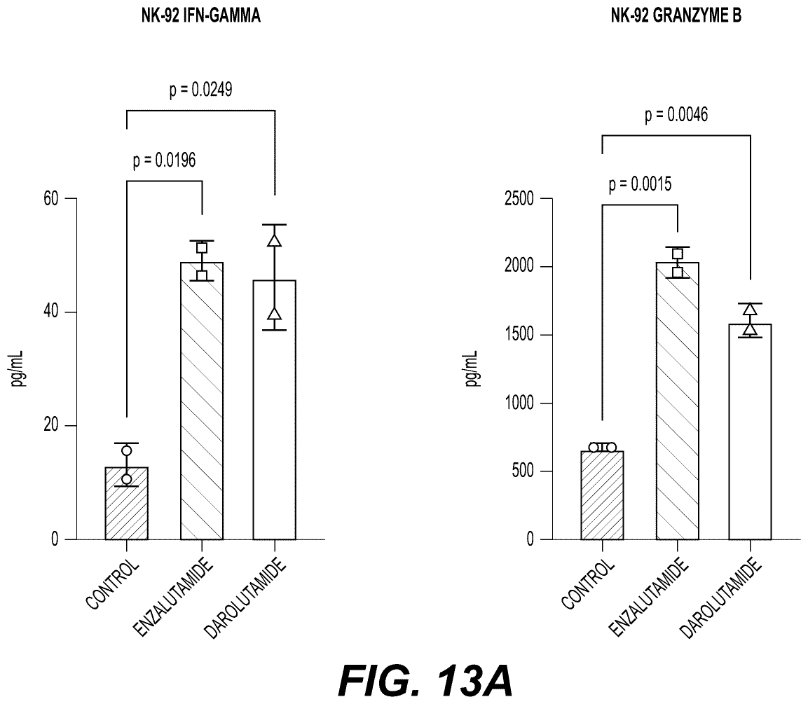

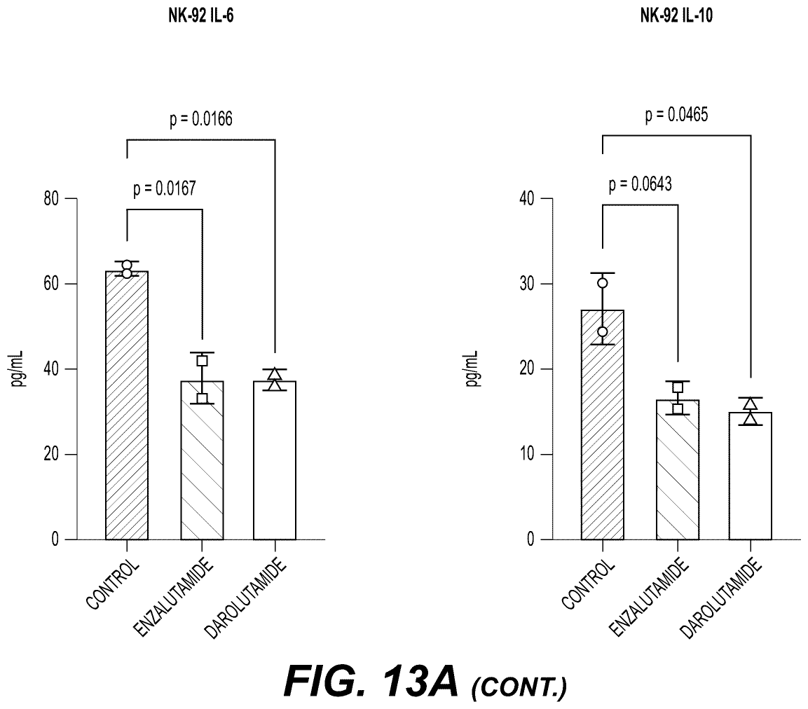

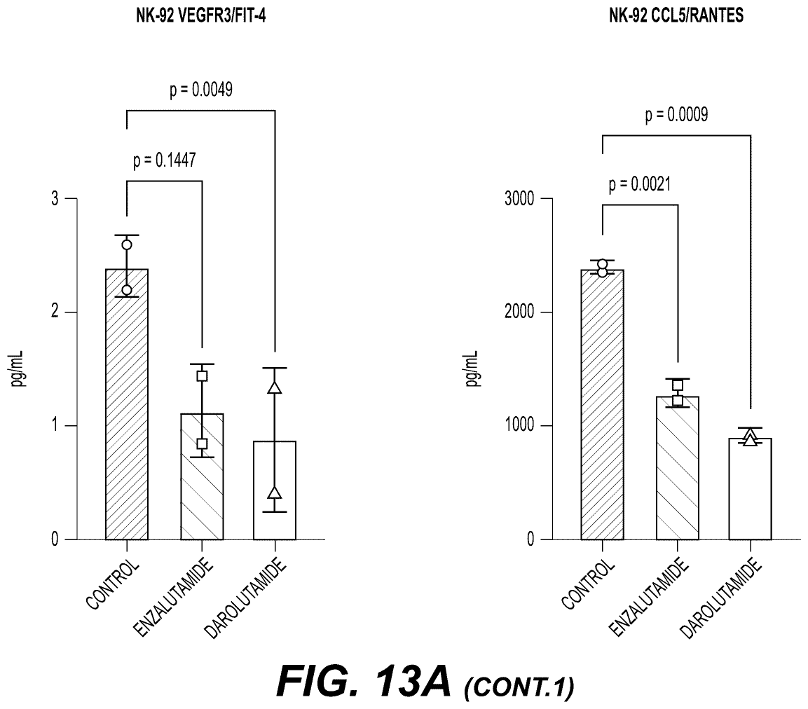

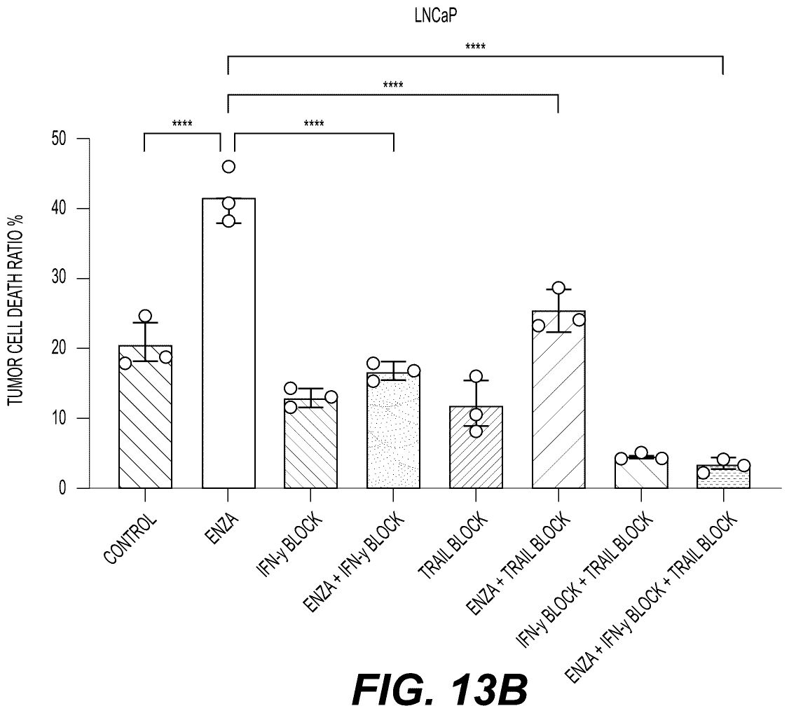

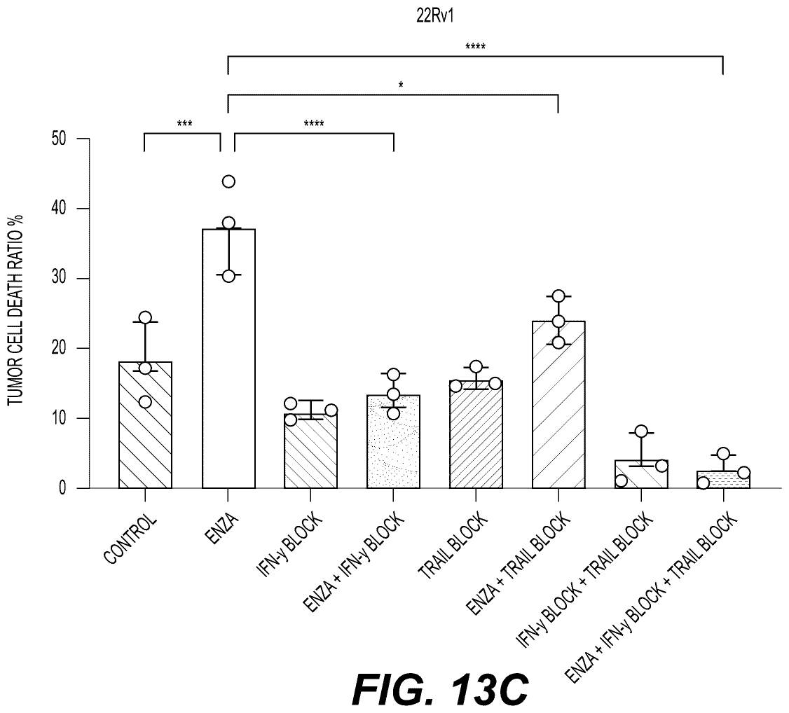

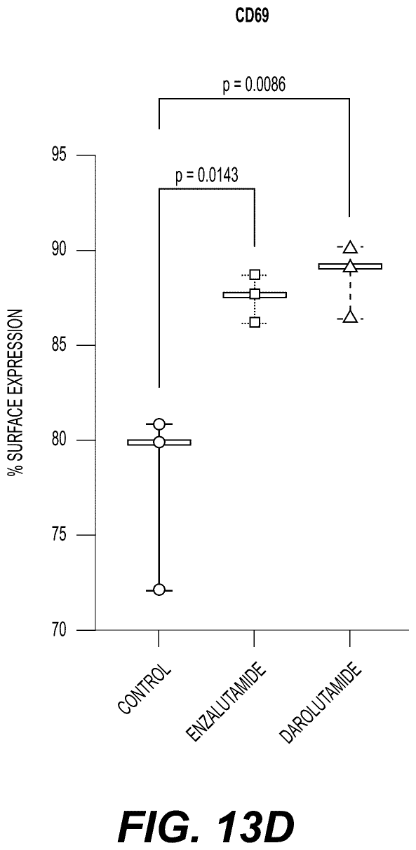

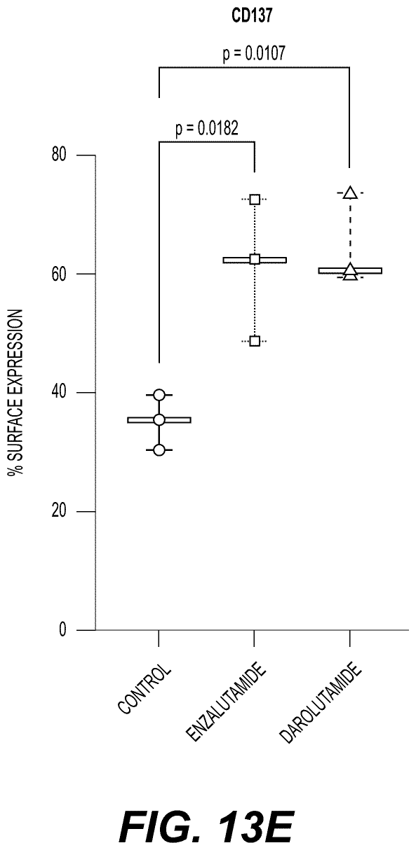

Daro and enza increased NK-92 cell secretion of IFN-γ ([C]: 12.98 pg/ml±3.4; Enza: 48.88 pg/ml±3.18; Daro: 45.6 pg/ml±9.4, p=0.01) and granzyme B ([C]: 676.6pg/ml+36.2; Enza: 1027 pg/ml±105.5; Daro: 1599 pg/ml±118.6, p<0.001), and decreased the secretion of the immunosuppressive cytokine IL-10 ([C]: 27.3 pg/ml±4.16; Enza: 16.6 pg/ml±2.01; Daro: 15.24 pg/ml±1.48, p=0.04) (FIG. 13A 1-6). These results supported the role of ARi inducing NK activation and enhancing cytotoxic function through IFN-γ secretion.

To investigate the role of IFN-γ in mediating this ARi-induced enhancement of NK cell killing, IFN-γ-neutralizing monoclonal antibody (mAb) (10 μg/ml) was combined with ARi in co-culture experiments. Doses of the α-IFN-γ mAb were based on previous experiments and co-culture assays. Blocking IFN-γ reversed the immune enhancement effect of enzalutamide (FIG. 13B-C). Based on the upregulation of soluble ligands TRAIL-R2 and R3 by ARi on our cytokine analysis (data not shown), we investigated the dual blockade of IFN-γ and TRAIL with RIK-2 (TRAIL-CD253 neutralizing mAb).

Treatment of PCa and NK cells in the presence of enza, α-IFN-γ mAb, and RIK-2 reduced the NK cell-mediated killing of PCa cells (FIG. 13B-C). In the presence of enzalutamide treatment, the blockade of TRAIL diminished the immune enhancement killing, but not to the same degree as IFN-γ mAb blockade alone (FIG. 13B-C). The dual blockade, targeting TRAIL and IFN-γ, diminished NK cell PC cell killing almost entirely, even in the presence of ARi (FIG. 13B-C).

The surface expression of the NK cell activation markers CD69 and CD137 were also evaluated following the treatment of NK cell monocultures with ARi (FIG. 13D-E). Enza and daro increased the expression of CD69 and CD137 (CD69 [C]:77.7%±4.75, enza: 87.4%±1.4, daro: 88.63%±1.94, p=0.009; CD137 [C]:35.1%±4.77, enza: 61.54%±12, daro:64.87%±7.96, p=0.01).

ARi modulates apoptotic and antiapoptotic proteins as well as pro and antitumorigenic cytokines in PCa cell lines

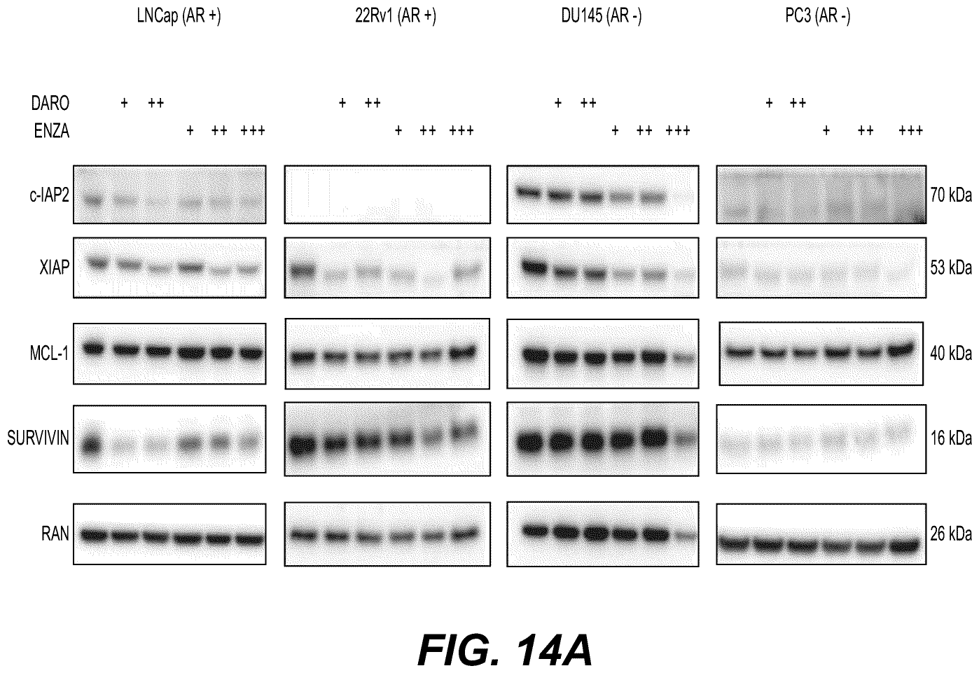

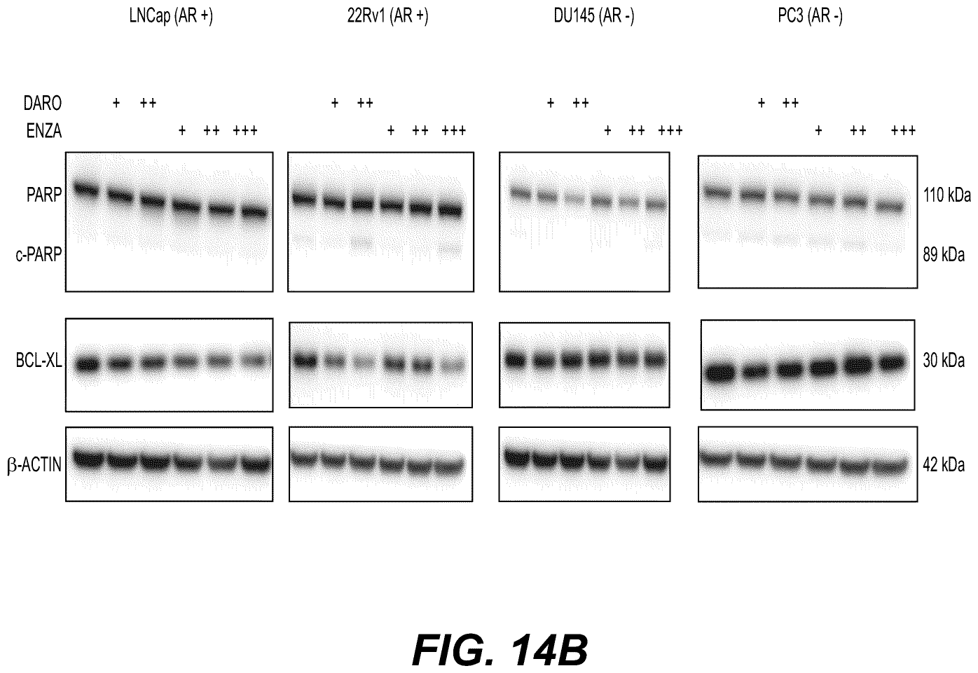

We investigated the effects of ARi on PCa cells' expression of Bcl-2 proteins and cytokine secretion to assess a possible priming effect of treatment, making these cells susceptible to NK cell killing. Enzalutamide (5, 10, and 15 μM) and darolutamide (10, 20 μM) reduced the expression of the X-linked inhibitor of apoptosis (XIAP) in DU145, 22Rv1, and LNCaP cell lines (FIG. 14A-B). Western Blot analysis of apoptotic and antiapoptotic proteins on the PCa cell lines with darolutamide (10 and 20 μM) and enzalutamide (5, 10, 20 μM) for 48 h. Treatment with ARi on the PCa cell lines evidenced changes in both pro and antiapoptotic proteins, irrespective of ARi sensitivity. The changes in protein expression were not similar in the PCa cell lines and cannot explain a single mechanism of tumor cell priming for NK cell killing.

The PC3 cell line expressed lower levels of XIAP at baseline, which did not change with treatment. The reduced expression of XIAP was more pronounced with darolutamide than enzalutamide. Both ARi also reduced the expression of c-IAP2 (DU145 cells and LNCaP) and Bcl-XL (22Rv1). Survivin levels decreased with treatment only in LNCaP. There was no significant change in the expression of the antiapoptotic protein MCL-1 in any cell lines with treatment (FIG. 14A-B). These results suggest that ARi modulate anti-apoptotic proteins in both androgen-sensitive and resistant prostate cancer cell lines, potentially increasing their susceptibility to immune-mediated killing as previously shown for AR-sensitive tumor cells. Nevertheless, these changes were not consistent in all PCa cell lines, and some were of limited magnitude, making the interpretation of their functional impact on susceptibility to NK cell killing difficult.

We then investigated the cytokine profiles of prostate cancer cell lines after treatment with ARi. Each cell line was treated with 10 nM of DHT for at least 72 hours before treatment with enzalutamide or darolutamide. Treatment of PCa cell lines with enzalutamide and darolutamide for 24 h increased the secretion of immune-activating cytokines such as IL-2 and reduced the secretion of pro-tumorigenic and immunosuppressive cytokines such as VEGF, M-CSF, IL-8, CXCL10 and GDF-15. Additionally, DU145, 22Rv1, and PC3 cell lines had upregulation of IFN-γ. ARi downregulated soluble FasL and increased TRAIL-R2 and R3. Upregulation of GDF-15 by ARi was limited to ARi-resistant prostate cells (PC3, DU145, and 22Rv1). GDF-15 is implicated in the immunosuppressive prostate cancer TME and the progression of benign prostate hyperplasia to adenocarcinoma.

Blocking the NK cell receptor NKG2A with monalizumab potentiates the ARi-induced immune-mediated killing of PCa cells

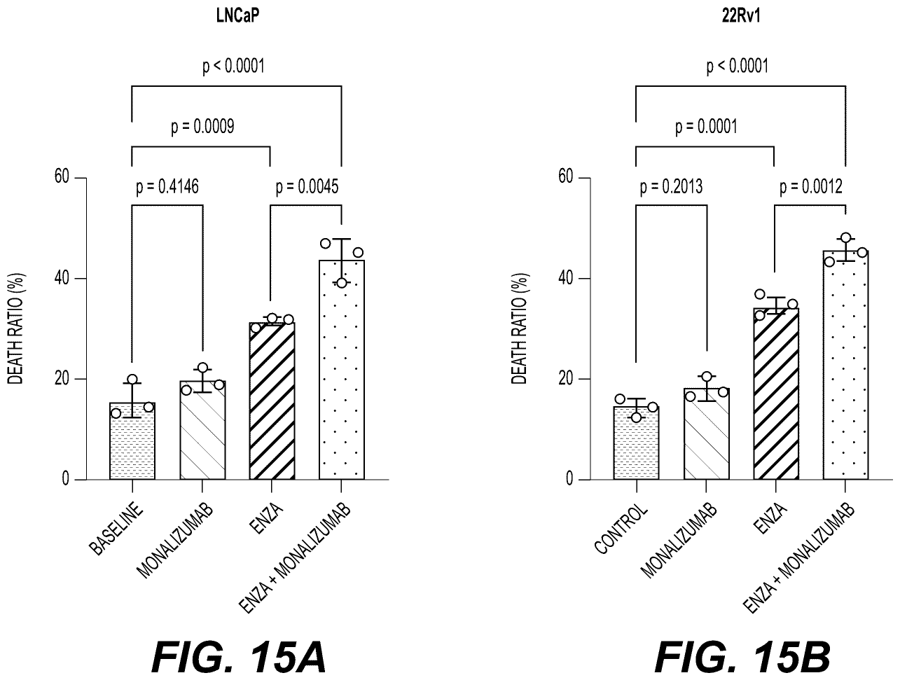

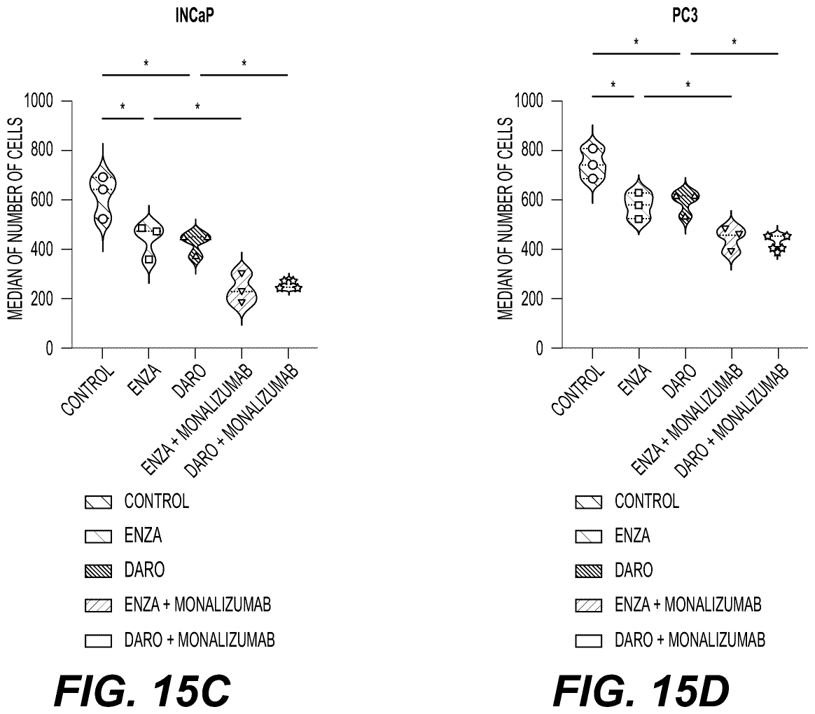

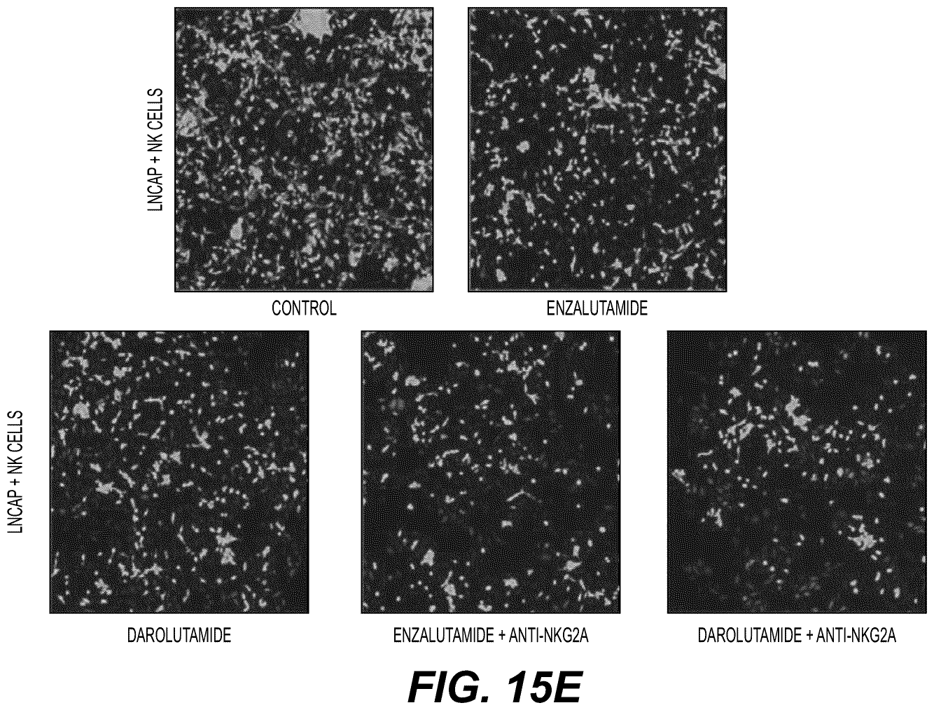

To evaluate a strategy to potentiate NK cell killing induced by ARi, co-culture experiments of PCa and NK cells were performed with enzalutamide combined with monalizumab, an NKG2A blocking mAb. Monalizumab significantly increased enzalutamide-induced immune enhancement irrespective of the ARi sensitivity of PCa cell lines. (FIG. 15A-B; LNCaP: [C] 15.89%±3.56, enza: 35.32%±1.09, monalizumab: 19.89%±2.4, enza+monalizumab: 45.97%±4.04, p=0.001; 22Rv1: [C]:14.23%±2.03, enza:34.84%±1.96, monalizumab: 18.29%±2.5, enza+monalizumab: 46.916%±2.4, p=0.001). Monalizumab as a single agent did not enhance NK cell killing (FIG. 15AB-or impact PCa or NK cell viability (data not shown).

To validate an additive effect of ARi and monalizumab enhancing NK cell cytotoxicity, we performed a co-culture assay with GFP-labeled PCa cell lines (including ARi-sensitive LNCaP and ARi-resistant PC3) and patient-derived NK cells. The NK cells were negatively selected from whole blood from patients with metastatic prostate cancer before initiation of androgen deprivation therapy. The addition of enzalutamide or darolutamide enhanced the cytotoxicity of NK cells, and the combination of ARi with monalizumab potentiated immune killing (FIG. 15C-D). Even though this effect was evident irrespective of the AR status of the cell line, the ARi-sensitive cell line, LNCaP, displayed greater sensitivity to the addition of monalizumab in combination with enzalutamide or darolutamide (FIG. 15C-D).

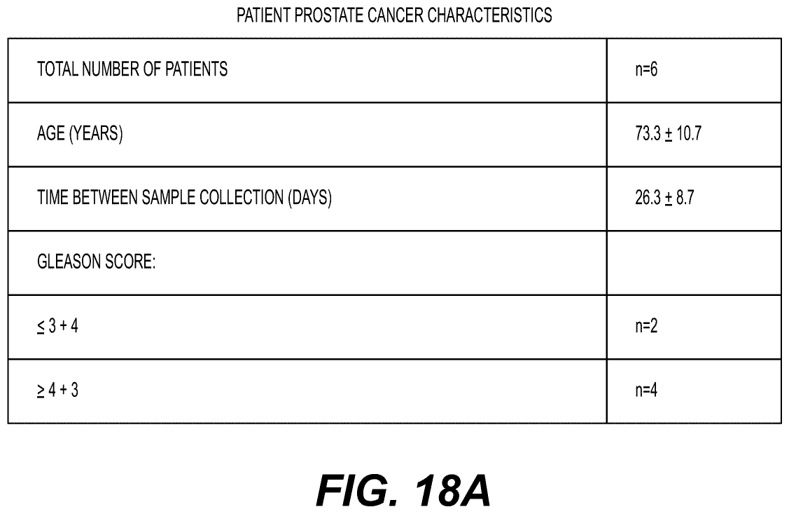

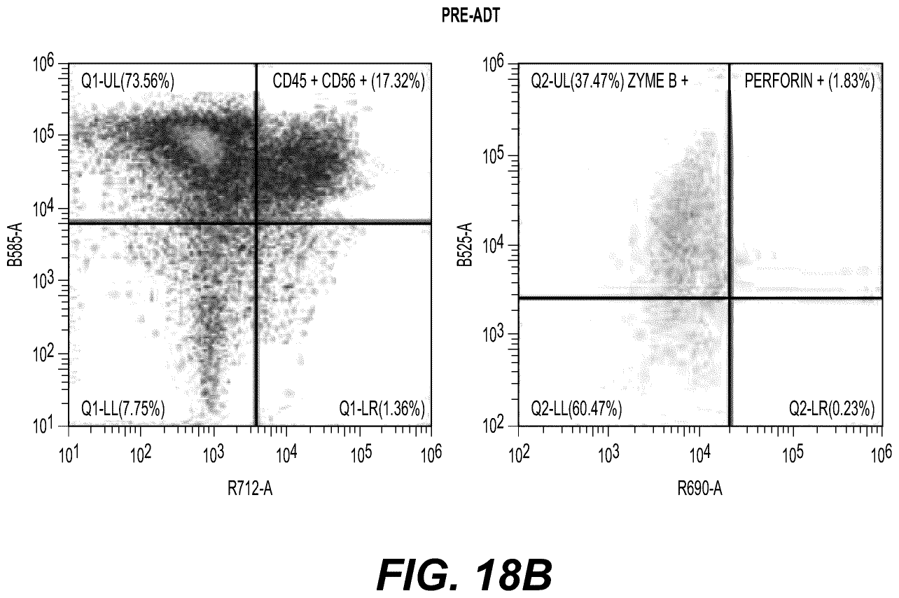

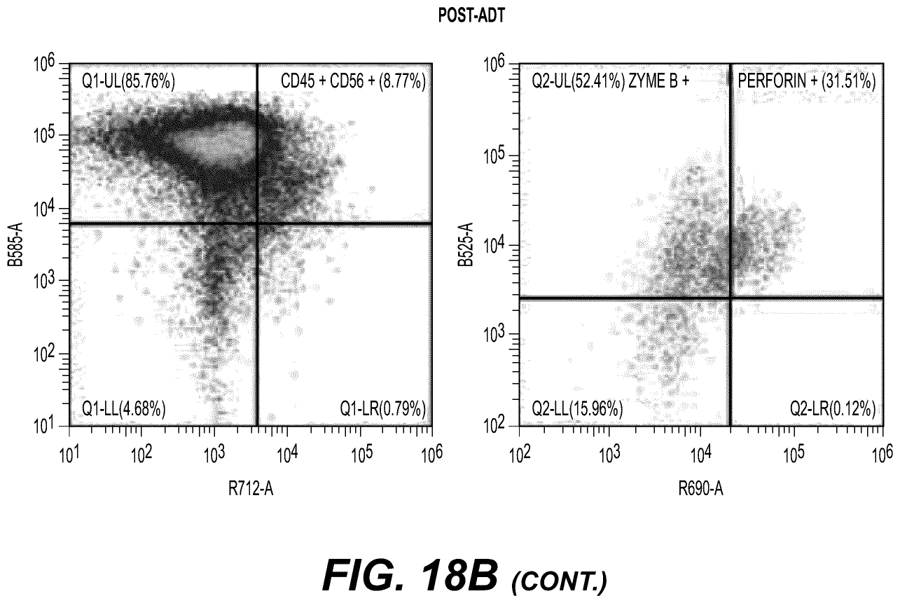

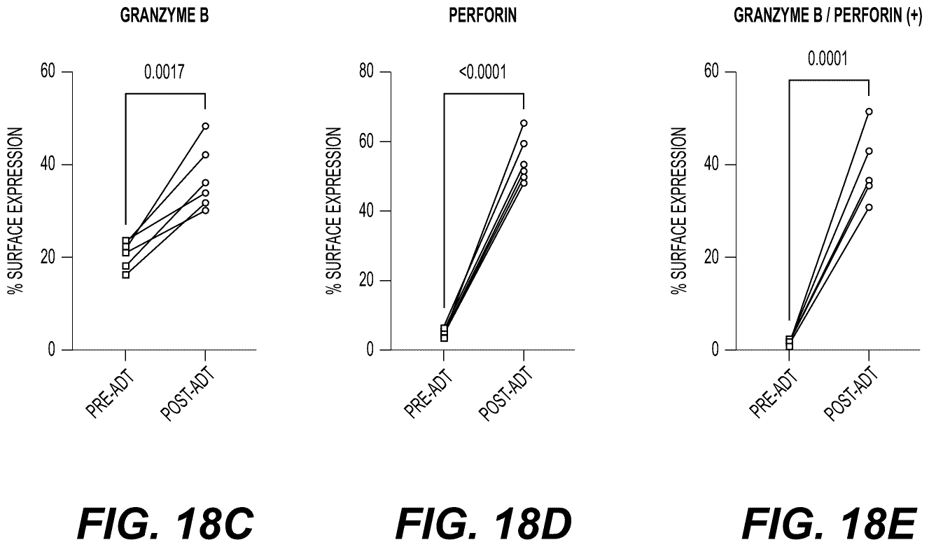

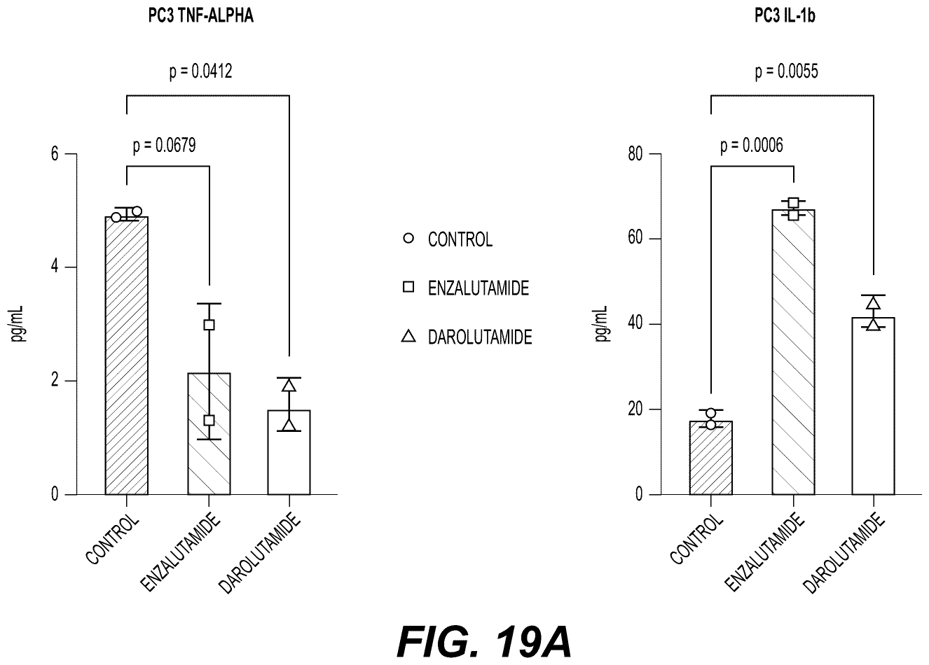

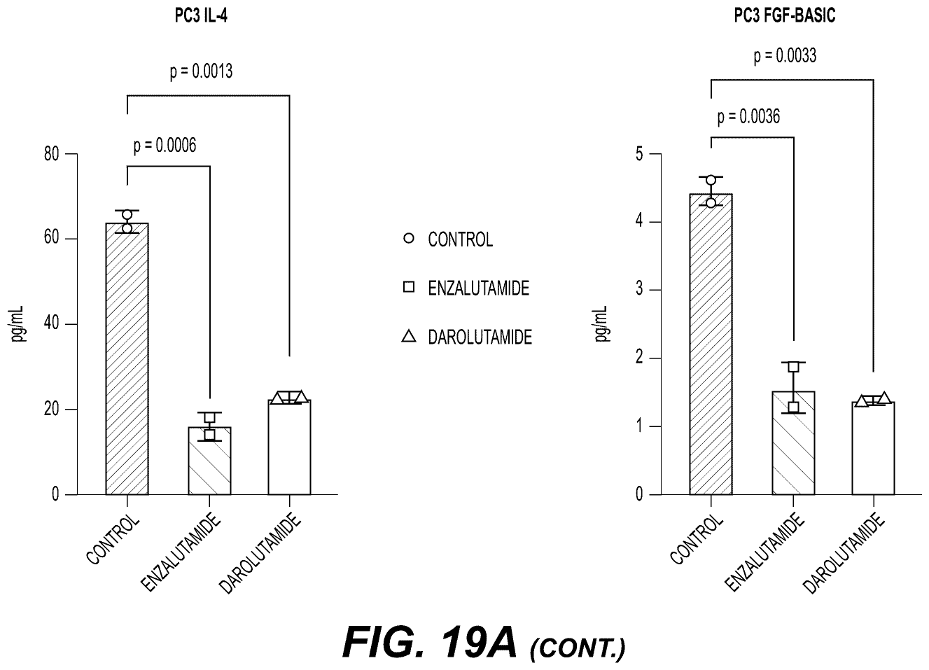

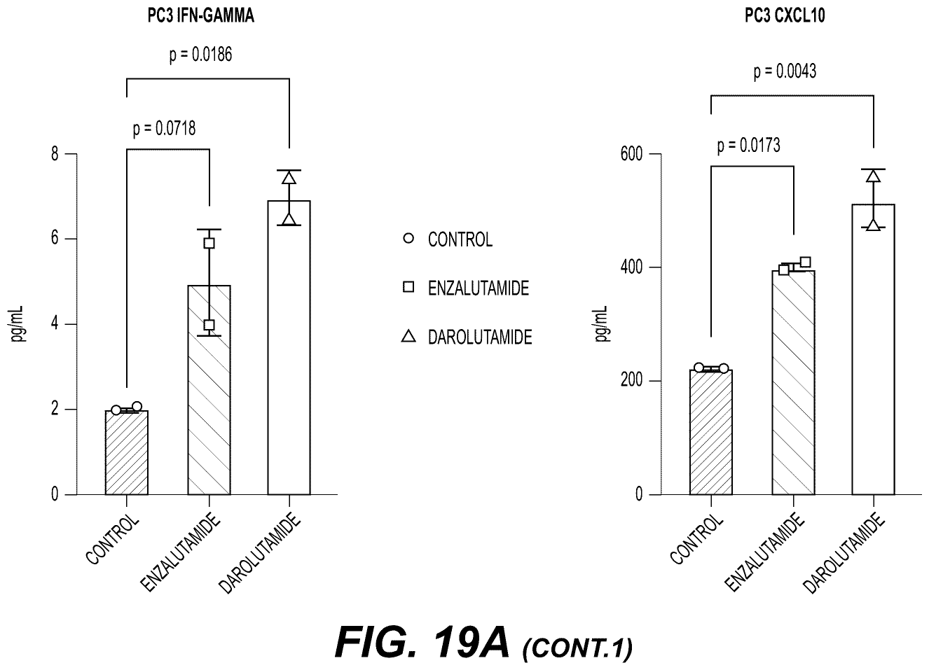

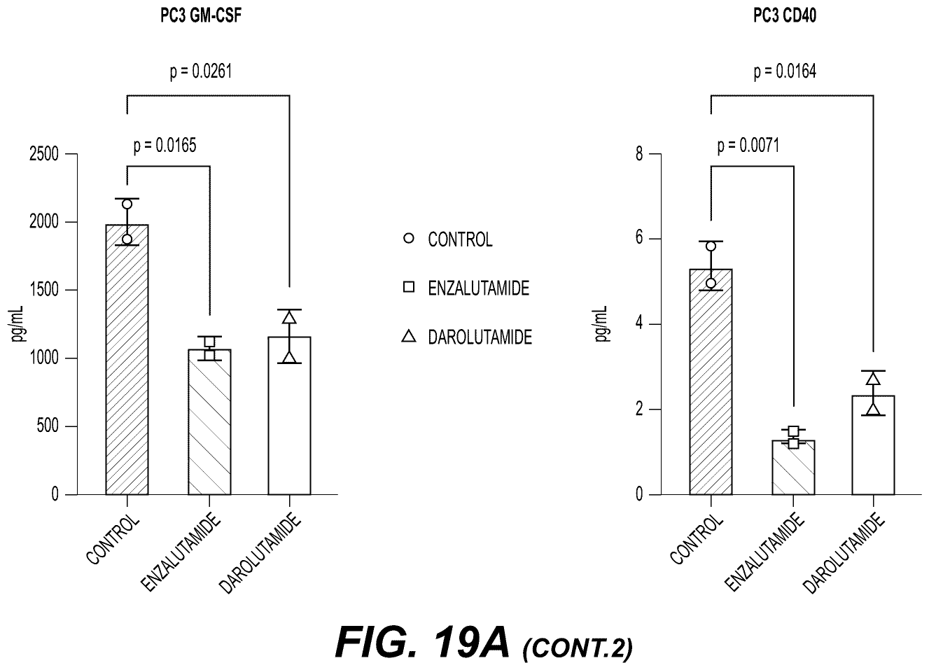

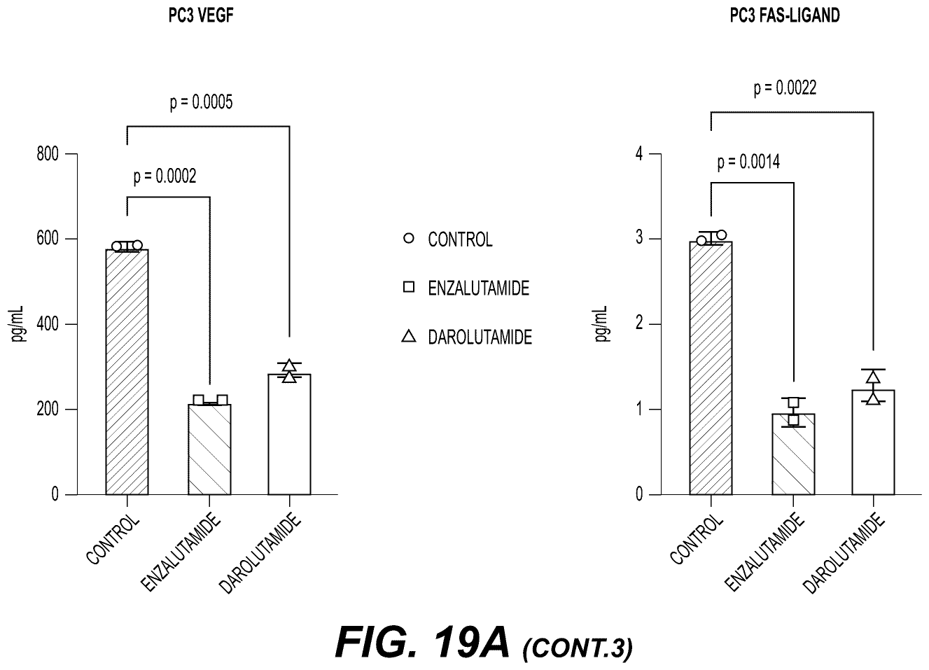

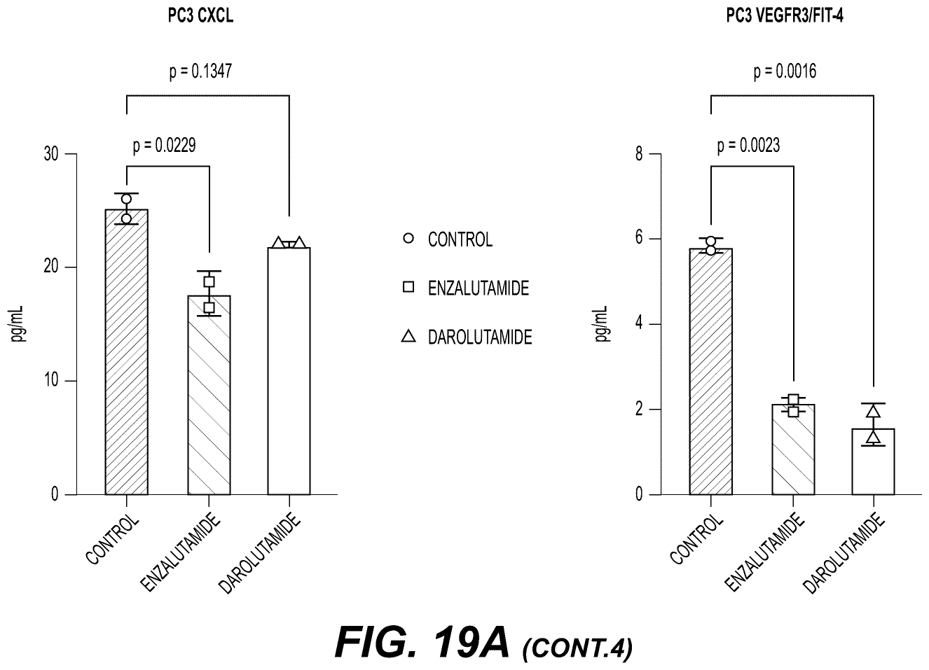

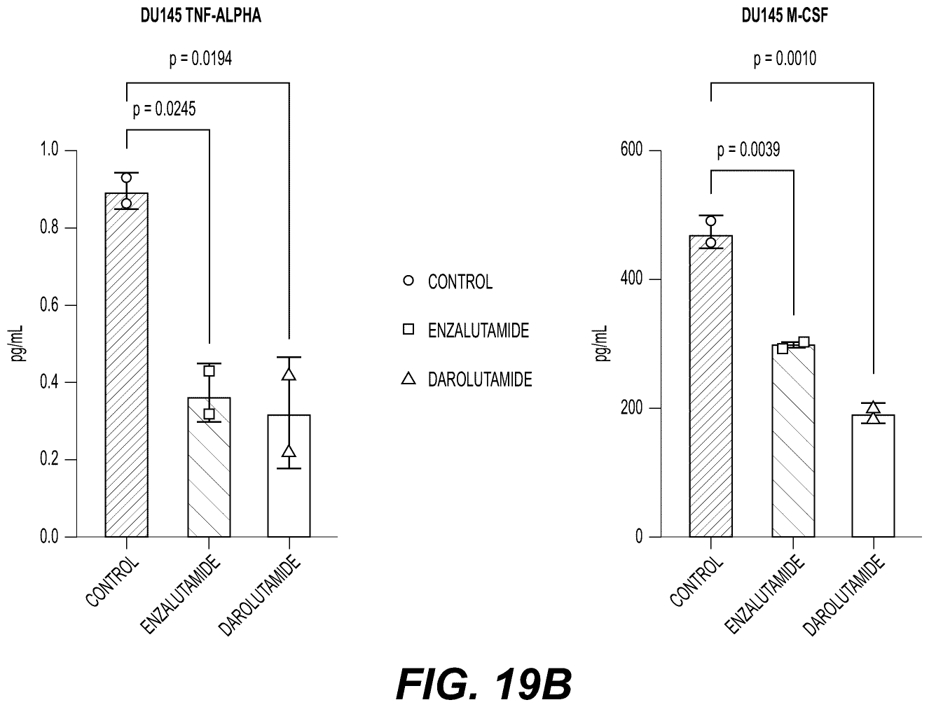

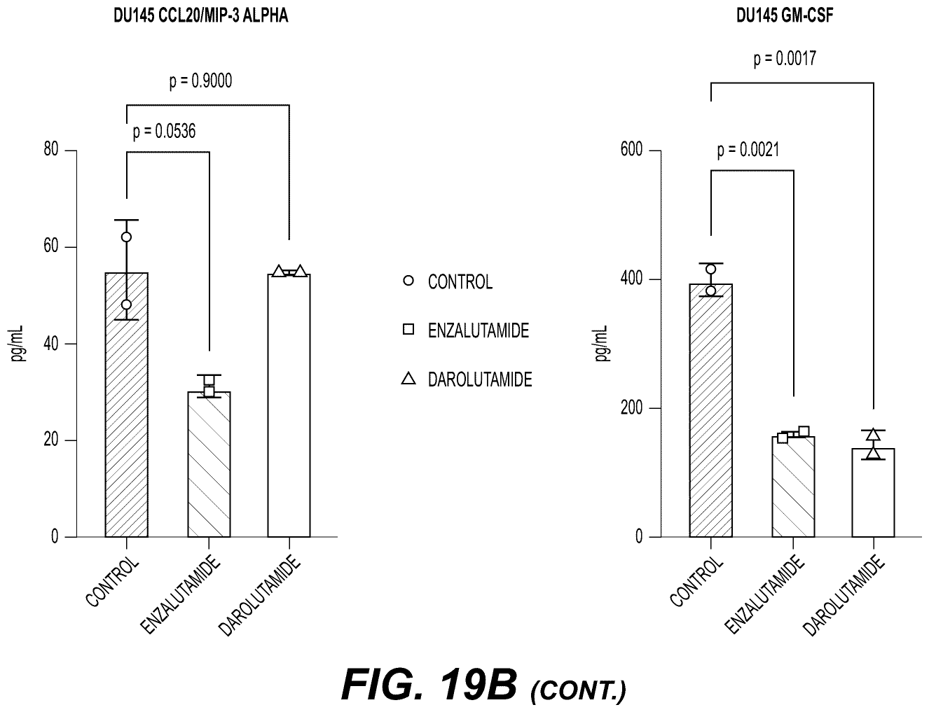

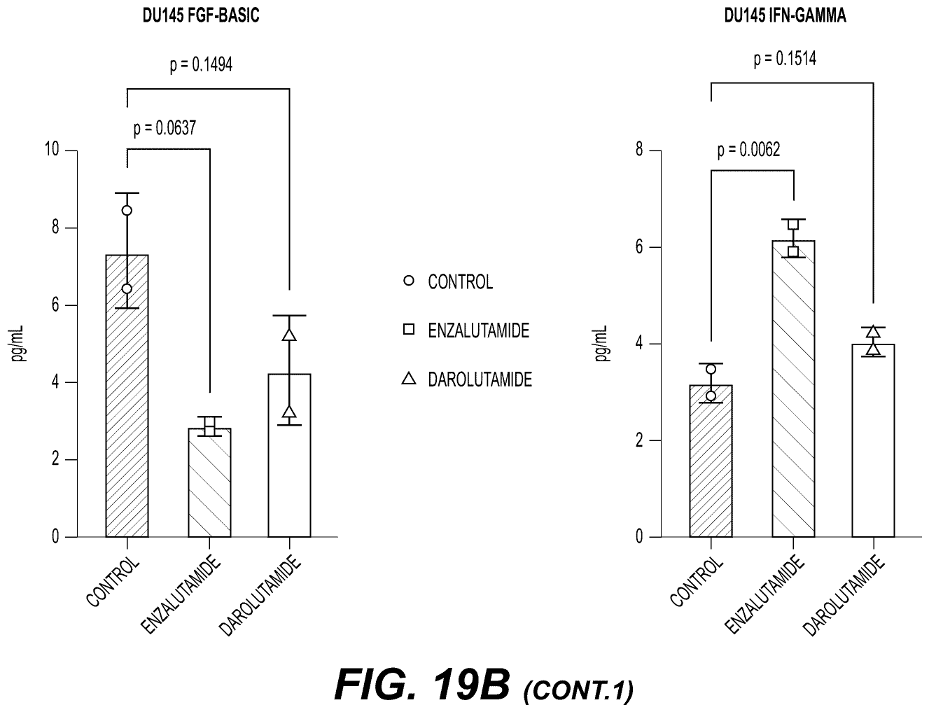

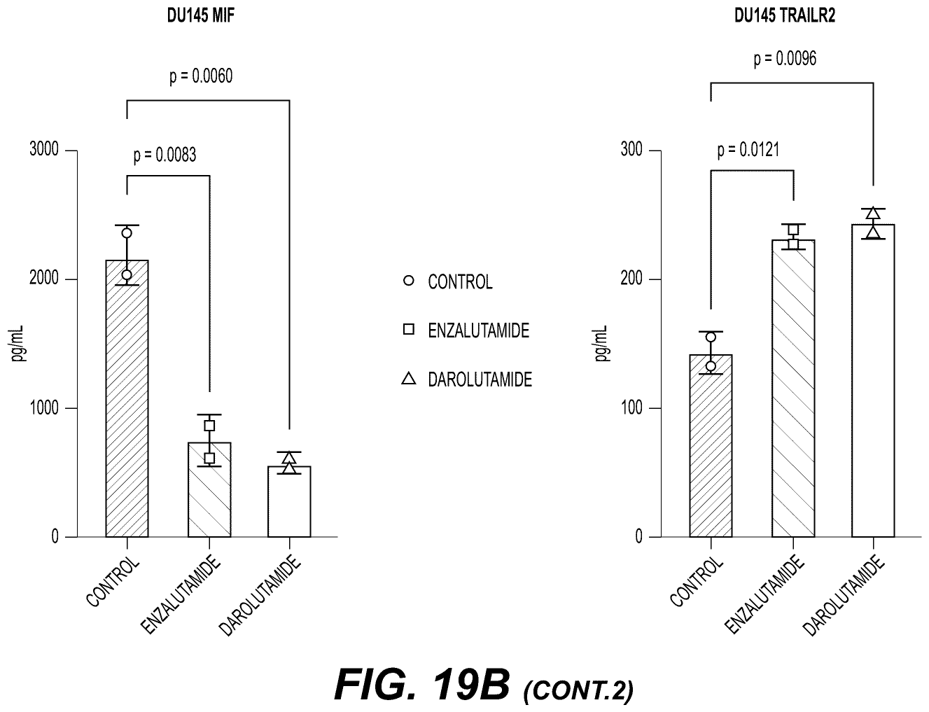

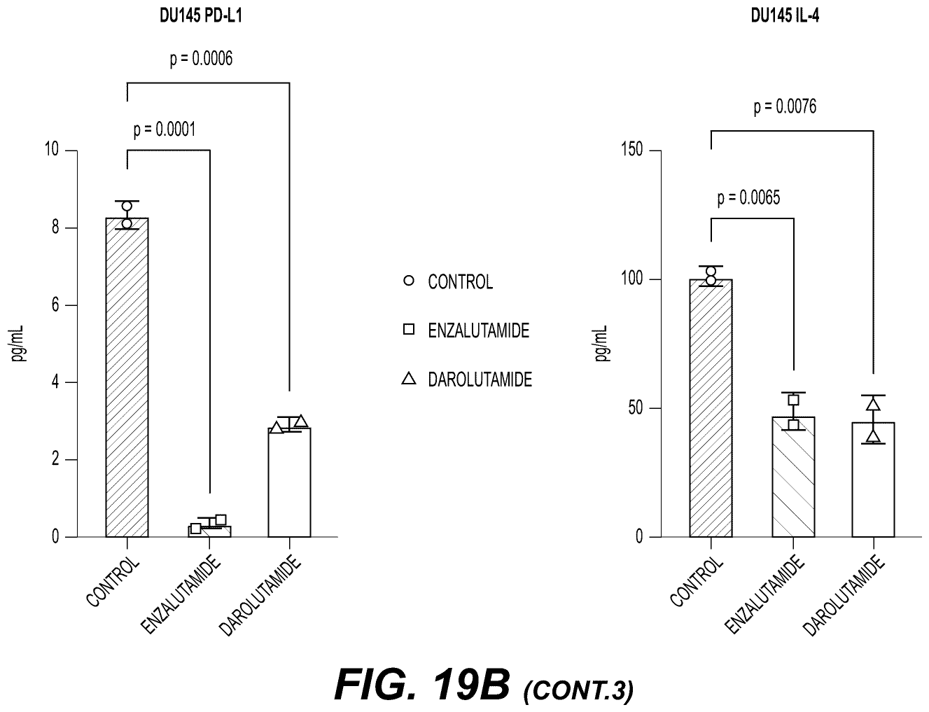

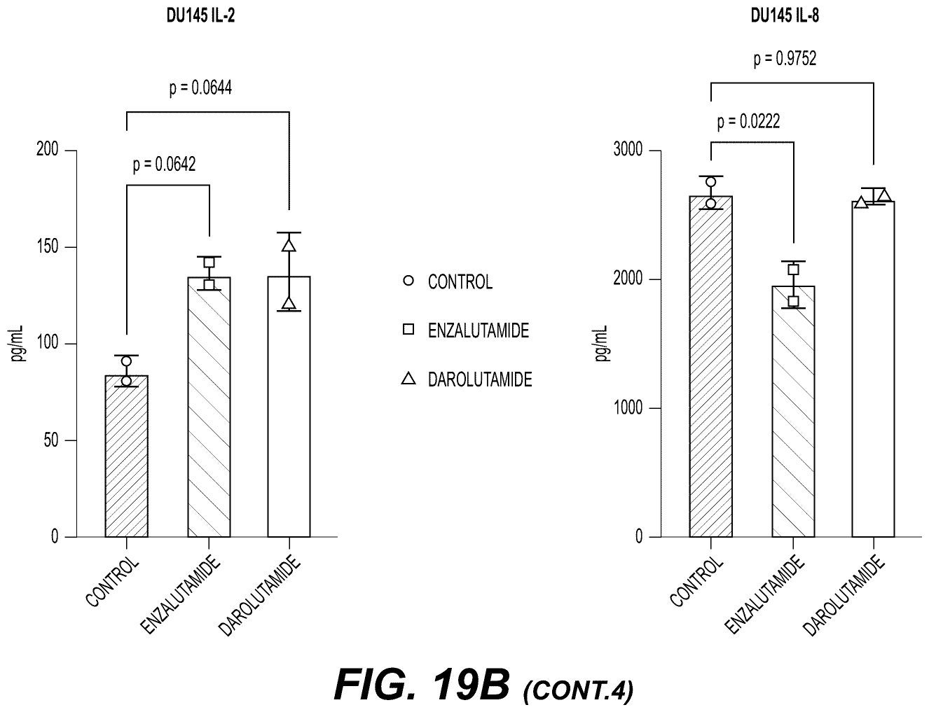

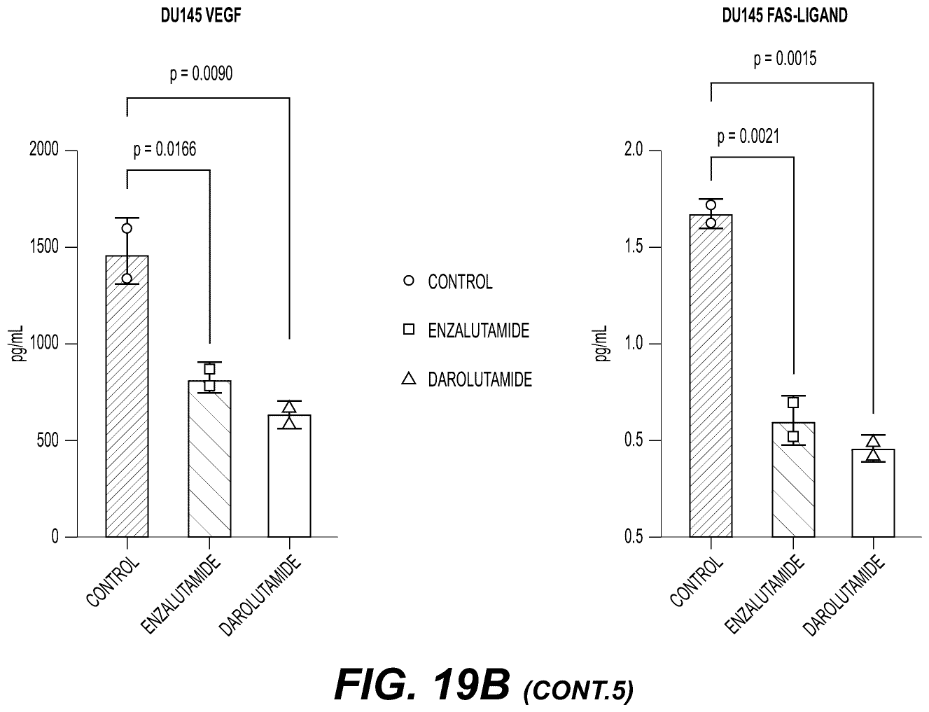

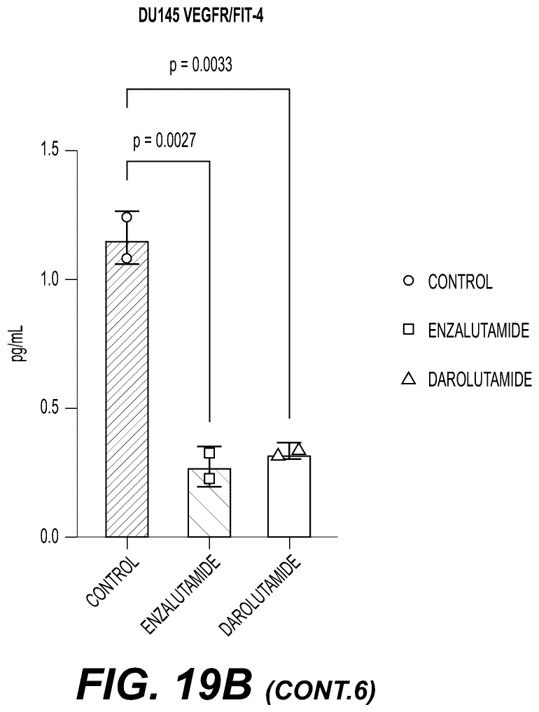

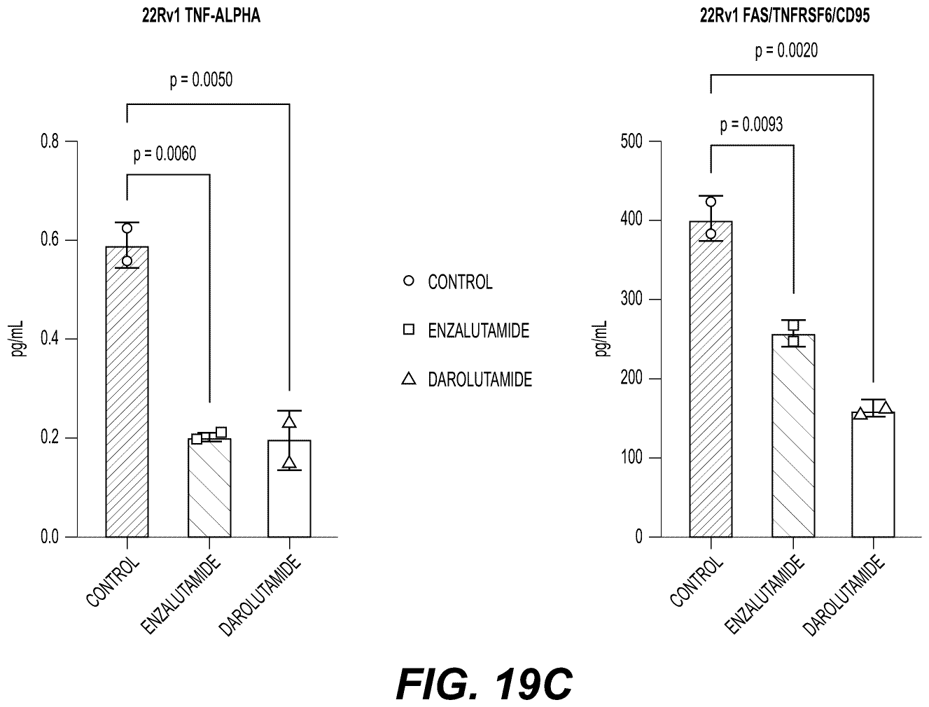

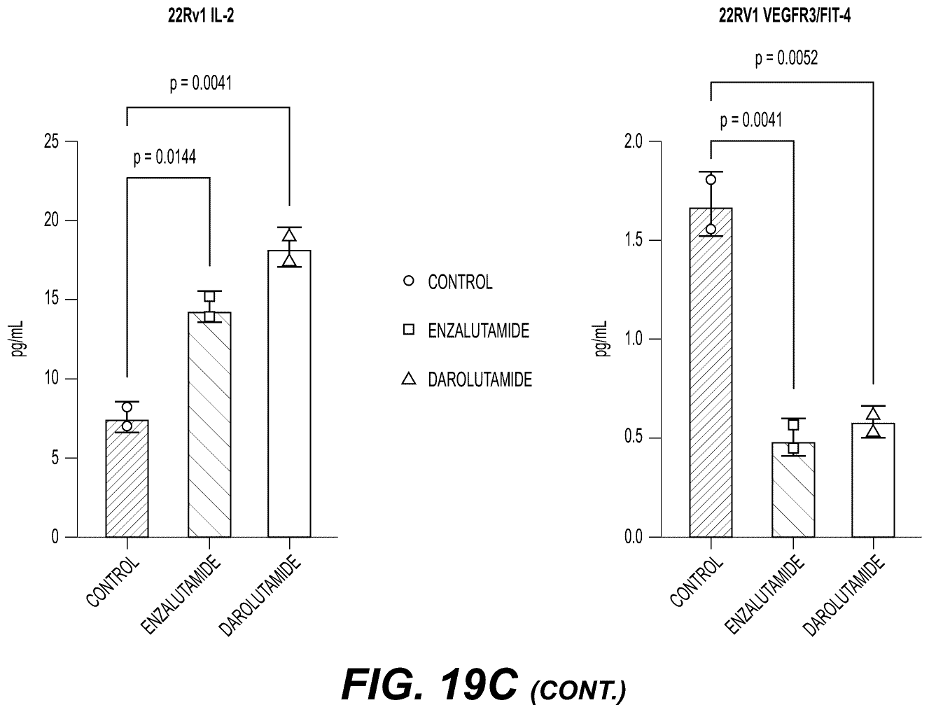

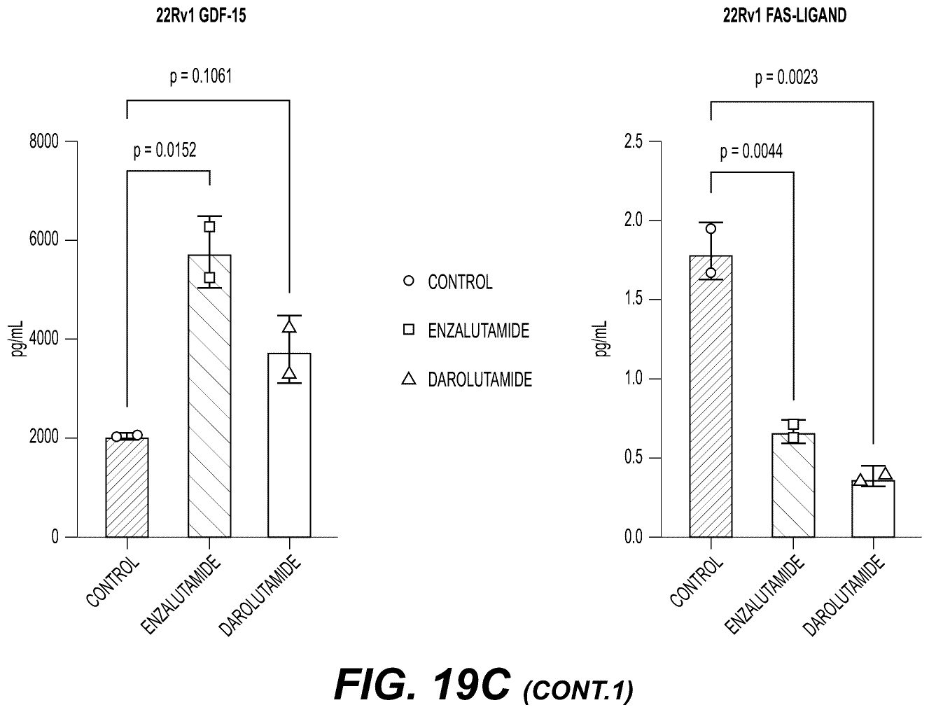

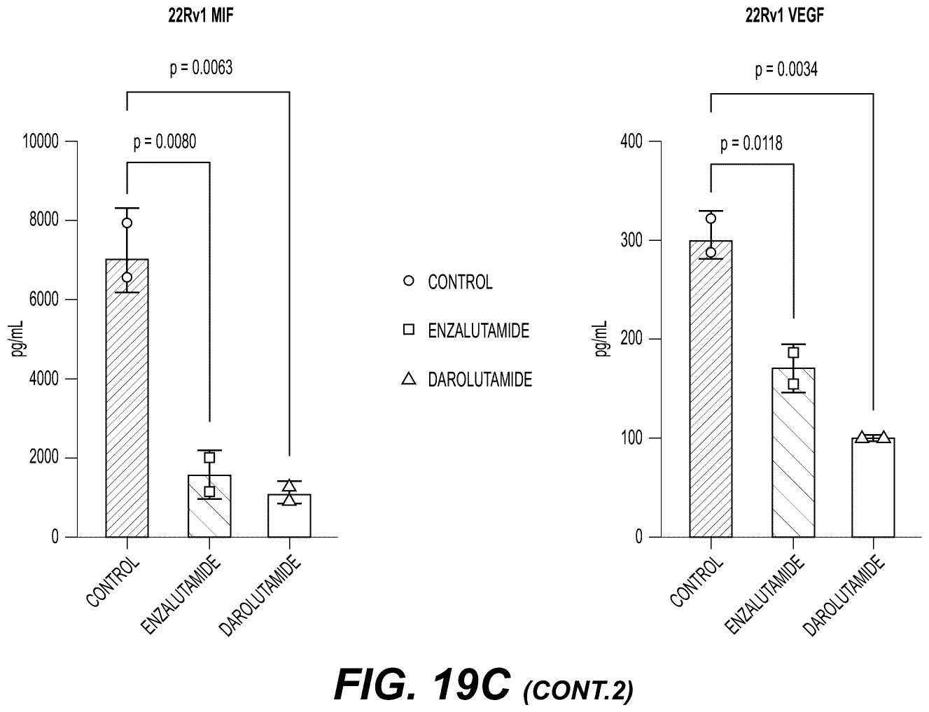

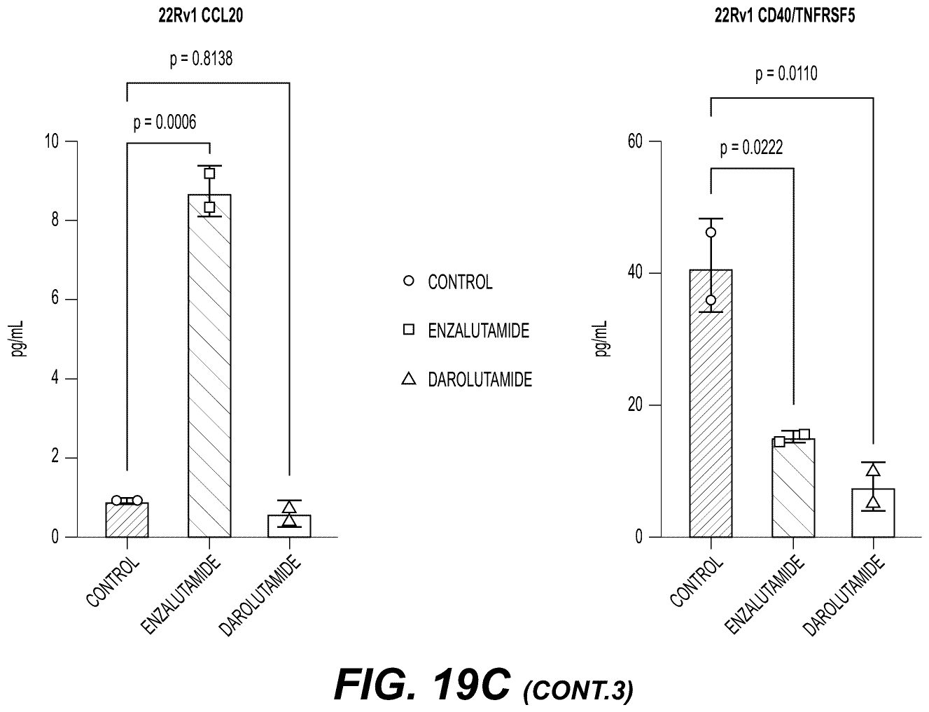

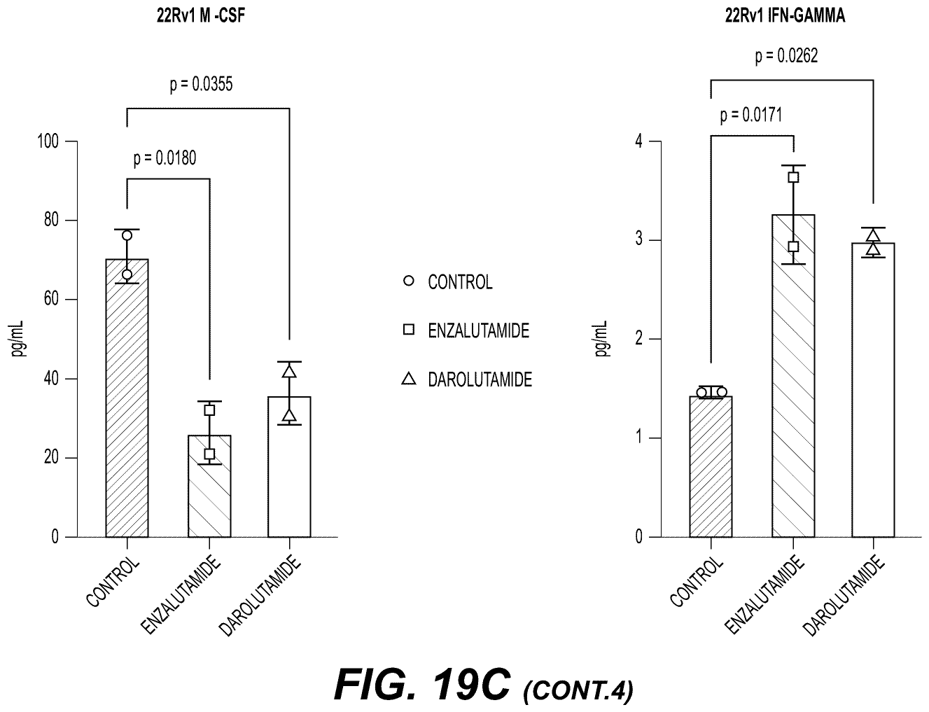

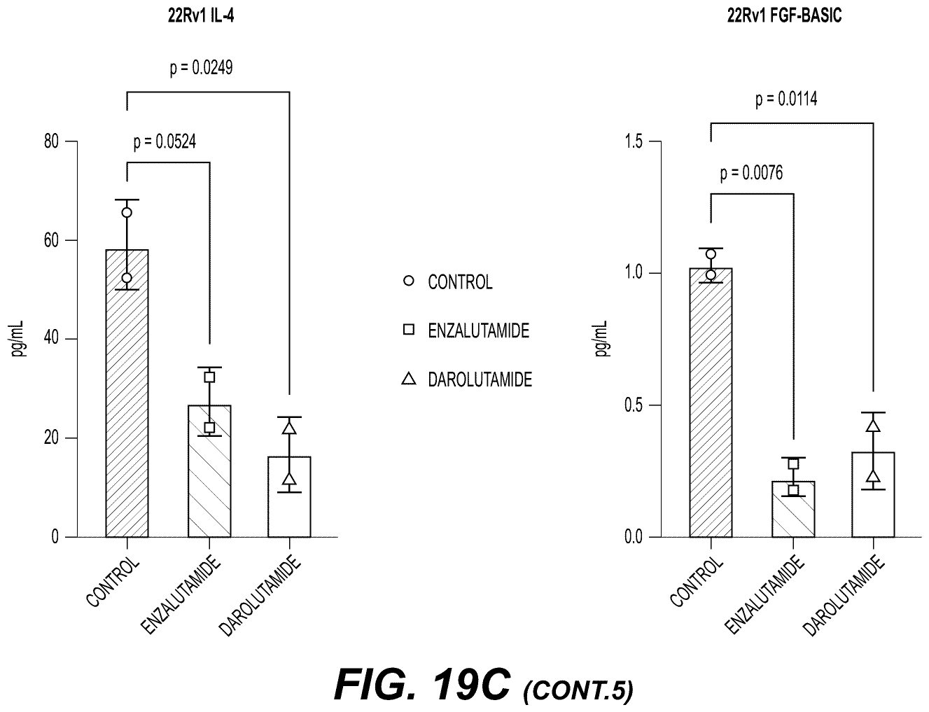

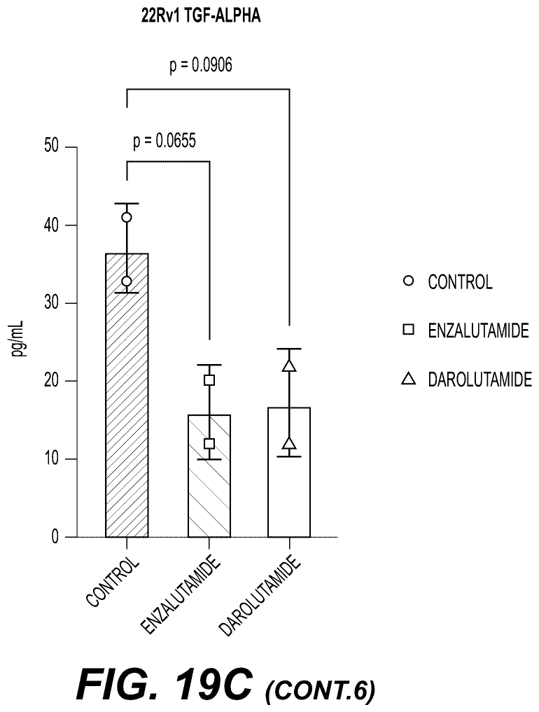

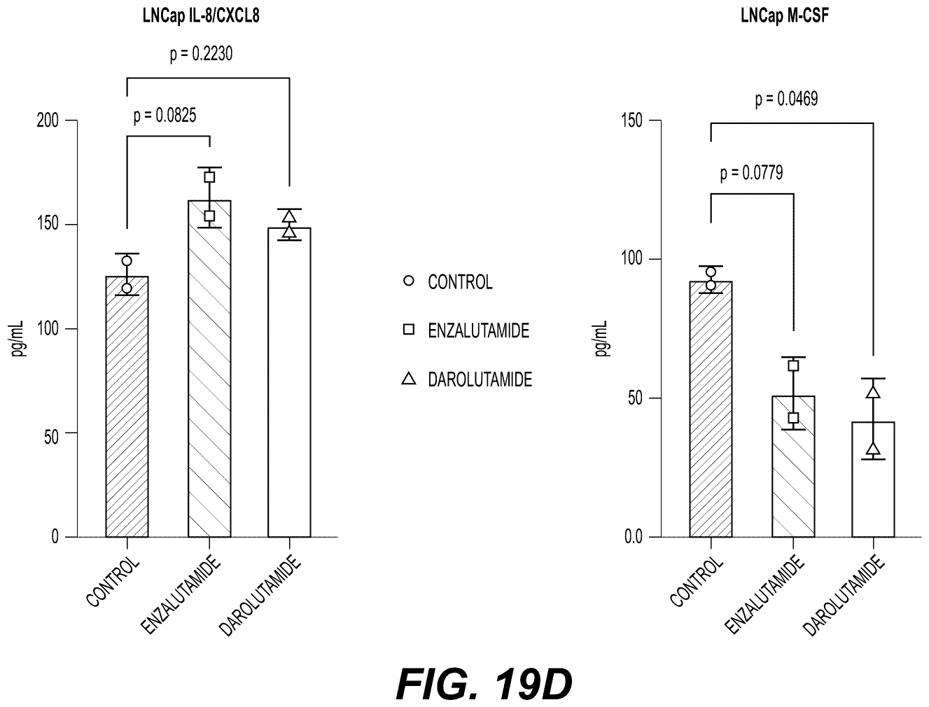

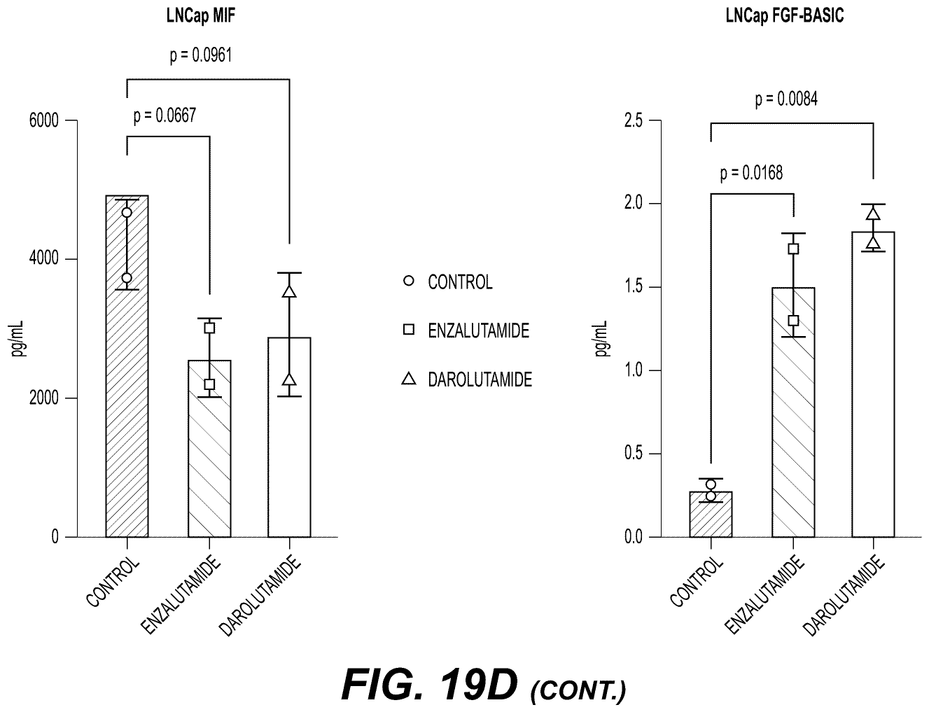

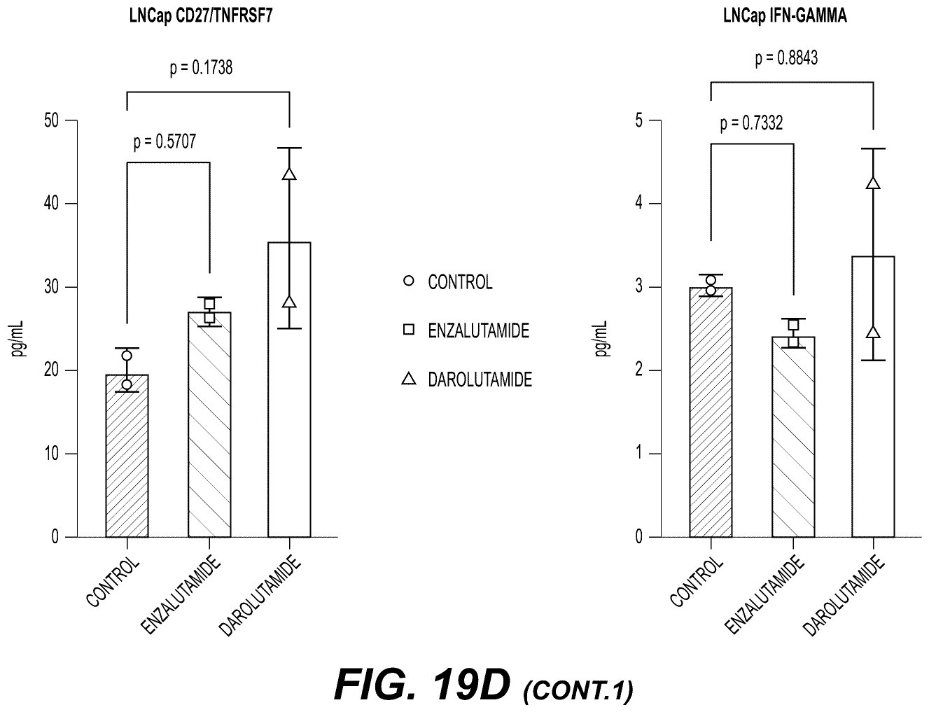

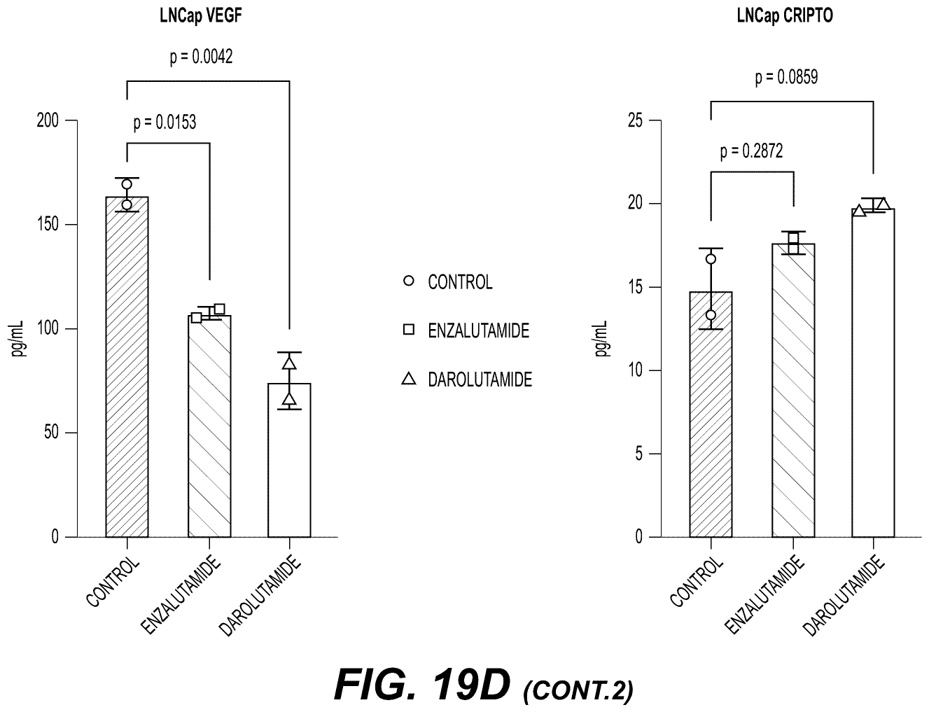

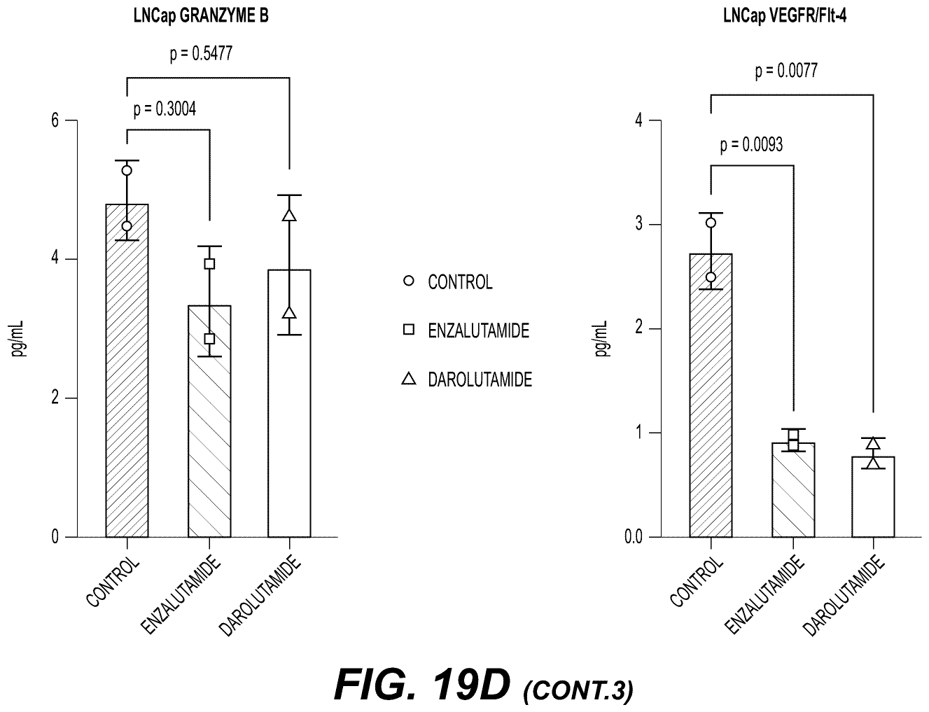

To investigate the effects of androgen deprivation therapy (ADT) on the activation of peripheral blood NK cells, blood samples were collected from patients diagnosed with castration-sensitive metastatic prostate cancer before initiation of ADT and approximately 26 days later when patients had achieved castration levels of testosterone (median time between sample collections: 26.3±8.7 days). Patient characteristics are summarized in FIG. 18A. Per standard of care, ADT was implemented with the administration of luteinizing hormone-releasing hormone (LHRH) agonists (e.g., leuprolide) or antagonists (e.g., degarelix). Patient-derived NK cells were co-cultured with PCa cells in a 1:1 ratio for 48 h and then analyzed by flow cytometry. After ADT, the patients displayed a greater number of granzyme B+ NK cells (pre-ADT: 20.53%±2.77; post-ADT: 37.24%±6.9; p=0.0017), as well as perforin+ NK cells (pre-ADT: 4.26%±0.92; post-ADT: 54.47%±6.47; p<0.001). The double-positive NK cell population (positive for granzyme B and perforin) also increased after ADT (pre-ADT: 3.54%±0.42; post-ADT: 38.15%±7.96; p=0.001) (FIG. 18C-E).

Androgen receptor blockade upregulates HLA-E in androgen-signaling sensitive PCa cells but not in androgen-independent ones.

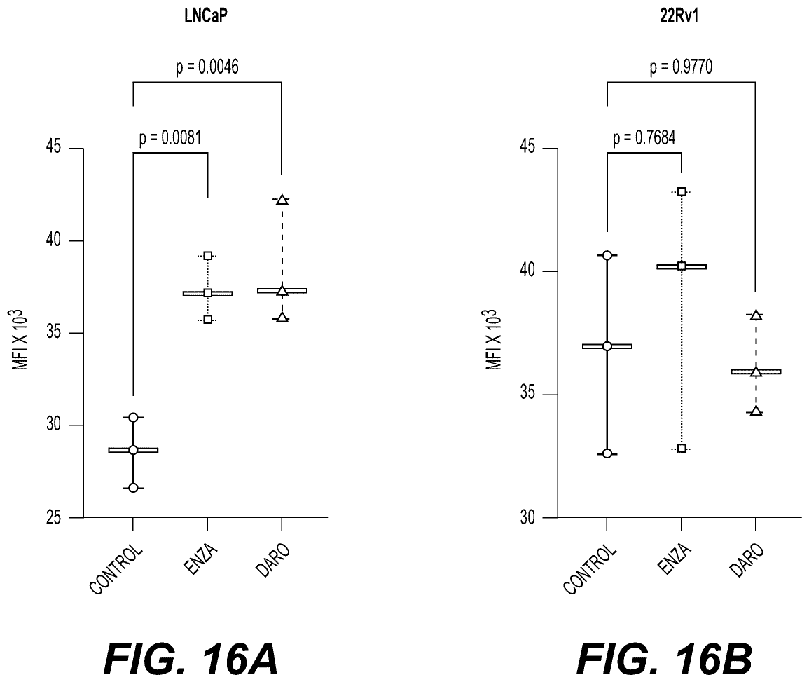

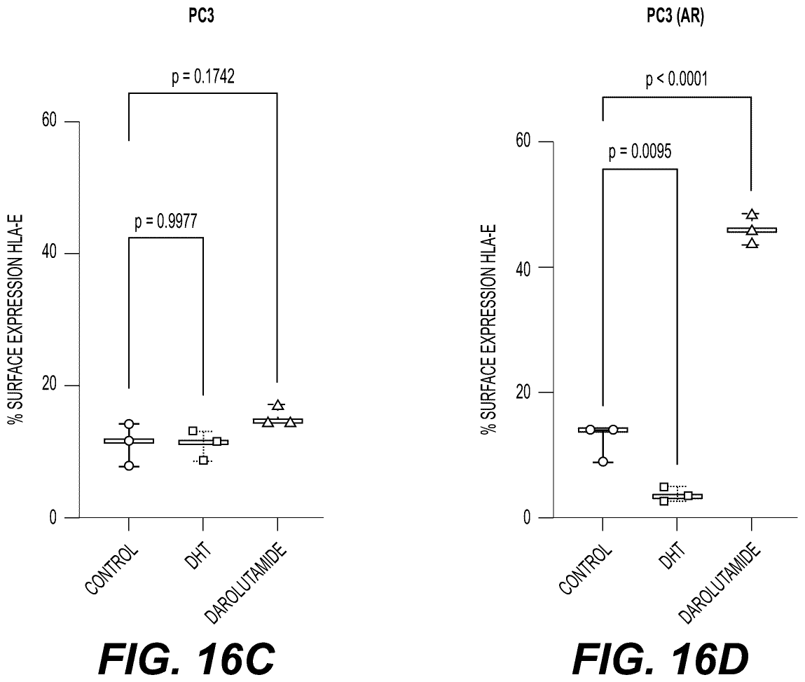

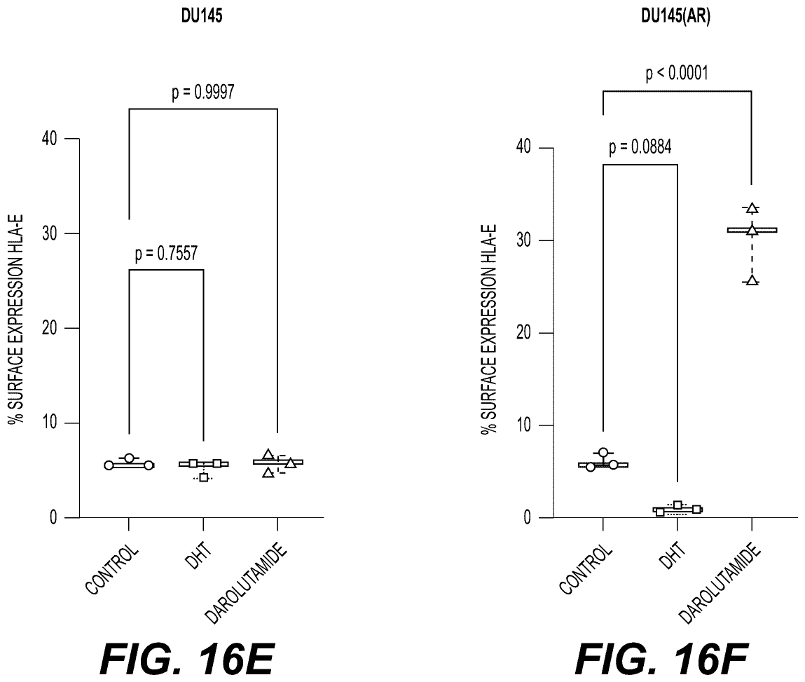

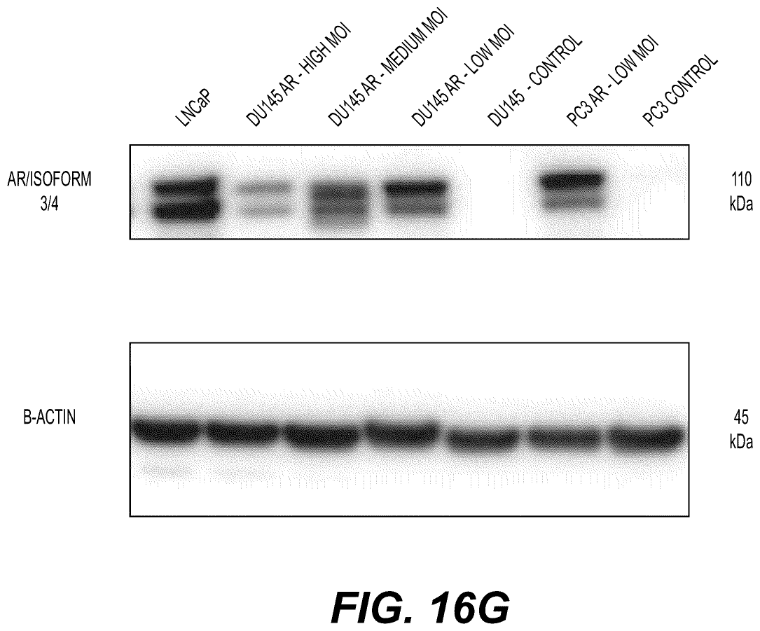

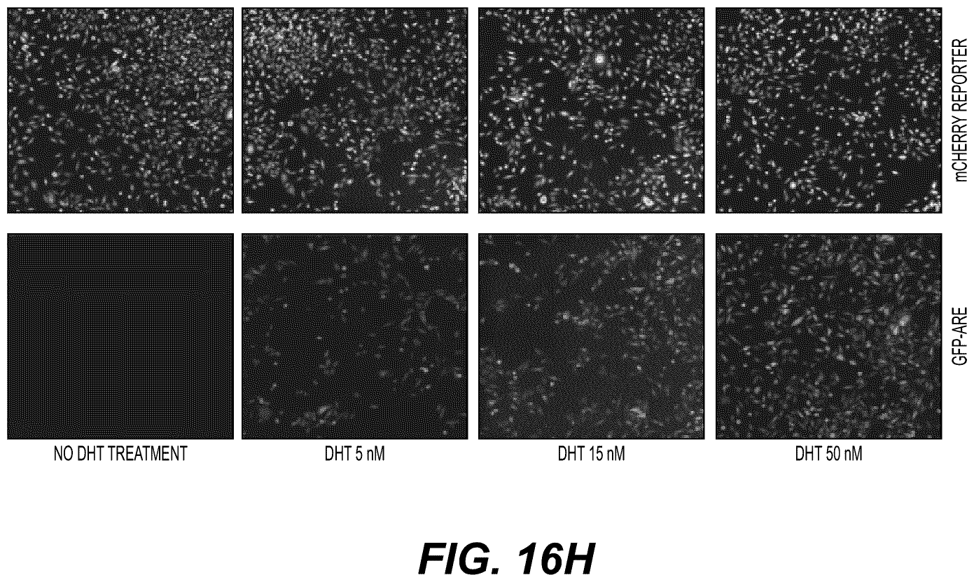

The effect of ARi on the NKG2A ligand, HLA-E, was investigated by flow cytometry on PCa cells treated with enza and daro. ARis increased the surface expression of HLA-E in LNCaP ([C]: 28.57±2.4×10³, enza: 37.38±1.7×10³ and daro: 39.47±3.39×10³, p=0.004) (FIG. 16A), and did not affect HLA-E expression of ARi-resistant cell line 22Rv1 ([C]: 34.76±5.1×10³, enza: 41.14+7.6×10³ and daro: 30.5±2.69×10³, p=0.14) (FIG. 16B).

To evaluate the modulation of the androgen receptor and tumor HLA-E expression, we astably transduced AR negative cell lines PC3 and DU145 with an Androgen Response Element (ARE) GFP reporter (FIG. 16H). After stably transducing these reporter-containing cells with an AR receptor (FIG. 16H), the transduced AR activity was confirmed by treating these AR+-constructed cell lines with DHT in different concentrations for 72 h. The expression and targeting of ARE were confirmed by increased expression of GFP+ cells (FIG. 16H). This system allowed us to evaluate AR-dependent signaling pathways. Using AR+ constructed cell lines, PC3 and DU145, we sought to determine the dependence of HLA-E expression and possible modulation by AR. As seen previously by the ARi resistant 22Rv1 cell line, non-AR-transduced cell lines did not alter their HLA-E expression (PC3 [C]:11.41%±3.16, DHT: 11.28%±2.19, Daro:15.47%±1.5, p=0.12, DU145[C]:2.06%±0.61, DHT: 1.84%±0.22, Daro:1.23%±0.6, p=0.11). Nevertheless, the AR-transduced cells showed decreased HLA-E expression upon ARi treatment, similar to what was seen with the AR-sensitive LNCaP cell line described previously (FIG. 16C-F). This could be a possible AR-dependent mechanism by which the treatment with ARi in combination with an NKG2A blocking strategy yielded a better killing profile in the presence of NK cells than the AR-insensitive ones. Although potential therapies targeting immune enhancement are being explored, the expression of exhaustion markers such as PD-1, NKG2A, and potential engaging ligands that diminish their cytotoxic function limits their application. Exploring these factors with new combination approaches, such as ARi and anti-NKG2A, and understanding the modulation of its targets upon treatment is key in overcoming immune-resistant scenarios, especially in prostate cancer.

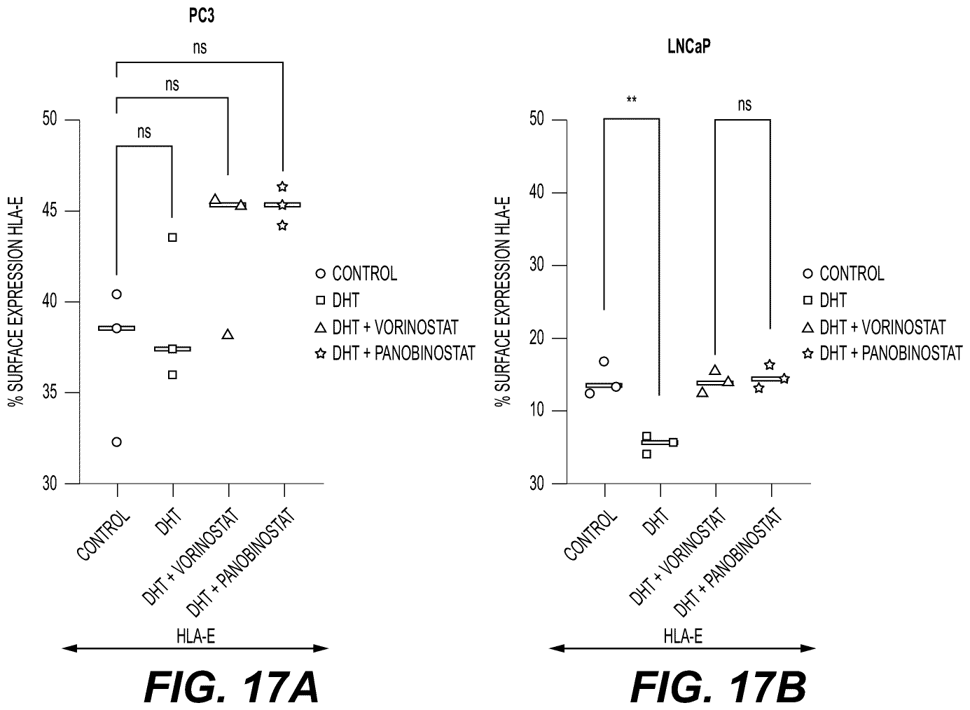

To explore a potential epigenetic regulation of HLA-E surface expression by androgen regulation, the HLA-E expression was evaluated on the PC3 and the AR-responsive cell line (LNCaP) after DHT treatment with and without pan-HDAC inhibitors vorinostat and panobinostat. Both cell lines were kept in culture for at least five days in the presence of 30 nM of DHT before the experiments (FIG. 17). The AR negative cell line, PC3, did not alter its surface expression of HLA-E upon treatment of DHT. The addition of HDACi did not change its surface expression (FIG. 17A). Experiments were conducted with noncytotoxic doses of vorinostat (0.3 μM) and panobinostat (3 nM). Interestingly, the expression of HLA-E on the AR responsive cell line (LNCaP) did not change significantly upon DHT treatment if cells were further treated with HDACi for 48 h (FIG. 17B). These results suggest that HLA-E regulation in the ARi-sensitive cell lines partly depends on epigenetic regulation.

Our results demonstrate that blockage of androgen signaling activates NK cells in vitro and enhances the killing of PCa cells in co-culture experiments. The AR inhibitors enzalutamide and darolutamide increased NK cell secretion of cytokines IFN-γ and TRAIL that mediated PCa killing. The blockade of IFN-γ inhibited this effect significantly, and the dual blockade of IFN-γ and TRAIL had further additive effects. Monalizumab, a monoclonal antibody blocking the immune inhibitory receptor NKG2A on NK cells, potentiated the NK cell cytotoxicity induced by ARi. The AR inhibitors also modulated PCa cells by decreasing the secretion of pro-tumorigenic factors (i.e., IL-4, TNF-a, VEGF, MIF, M-CSF, and GM-CSF) and downregulating anti-apoptotic proteins (XIAP, c-IAP2, and Bcl-XL). Our results also reveal a novel androgen-driven regulation of HLA-E expression in PCa cells. HLA-E is a nonclassical HLA class I molecule and the main ligand of the NKG2A receptor. HLA-E is frequently overexpressed in tumors and contributes to immune escape in the TME by inhibiting NK cells and NKG2A-expressing CD8 T cells. ARi-induced HLA-E expression in prostate cancer cells can protect them from NK lysis, contribute to a cold immune environment, and might represent a mechanism of resistance enabling the persistence of prostate cancer cells during treatment with AR inhibitors as well as ADT, a finding with significant clinical importance for the treatment of prostate cancer.

Our findings showing the immune-modulatory role of androgen signaling on NK cells and complement results describing the effect of ADT and AR blockade on T cell function. Building upon a clinical trial investigating the combination of anti-PD1 (pembrolizumab) with enzalutamide for treatment or mCRPC, Guan and colleagues showed that enzalutamide plus ADT significantly enhanced the anti-tumor effect of anti-PD-L1 in animal models compared to enzalutamide plus ADT. These effects were mediated by a direct effect of enzalutamide increasing T cell secretion of IFN-γ and granzyme B. The group demonstrated that the androgen receptor interacts with IFN-γ and granzyme B genes in open chromatin regions (OCRs) of memory CD8 T cells, blocking the rapid production of IFN-γ and Granzyme B enabled by these OCRs upon TCR stimulation. Blocking the androgen-mediated suppression of IFN-γ production in T cells with enzalutamide improved the anti-tumor response elicited by PD-L1 inhibition, helping overcome the relative resistance of prostate cancer to checkpoint inhibitors in vivo. Other results corroborate the androgen immune suppressive effects on T cells. ADT increased tumor infiltration of IFN-γ expressing T cells in the prostate TME. Enzalutamide activates IFN-γ signaling pathways and decreases the frequency of immunosuppressive cells in peripheral blood mononuclear cells isolated from patients with mCRPC. Our results show a direct effect of AR inhibitors increasing NK cell activation and secretion of IFN-γ, which can also impact T cell function through the functional crosstalk between these two cell lines, enabling an effective anti-tumor response. IFN-γ produced by NK cells promotes an anti-tumor T helper cell type 1 (Th1) polarization in CD4+ T cells. Activated NK cells stimulate CD4+ T cell proliferation by interacting with OX40/OX40L and indirectly influence T cell response by regulating differentiation of dendritic cells. NK cells can induce a type 17 polarization in CD8+ T cells, characterized by the ability to produce IFN-γ and IL-17A through priming of dendritic cells [30]. These findings suggest that therapeutic strategies targeting the activation of CD8 T cells and NK cells could overcome the immune suppressive TME in prostate cancer and lead to meaningful anti-tumor effects. One such strategy could be the combination of AR inhibitors plus monalizumab and anti-PD1/PD-L1 agents. Monalizumab is in clinical development for the treatment of lung cancer and other solid tumors in combination with durvalumab (anti-PD-L1) as a novel strategy to harness the innate immune response and activate NK cells, leading to clinically meaningful tumor response and immune infiltration. The importance of NK function in PCa and the therapeutic potential of targeting these cells is highlighted by results showing their impact on the clinical outcomes of patients. In a recent pan-cancer analysis, natural killer cell infiltration in PCa tumor specimens was associated with improved OS (HR 0.46, 95% CI 0.38-0.56, p=0.0001). The study also found that NK cell infiltration was associated with a 1.4-2.1-fold increased expression of immunomodulatory receptors LAG3 and TIGIT (p=0.0001). Another analysis of a large cohort of tumor specimens of patients with mCRPC showed that tumor infiltration by cytotoxic NK cells and higher expression of activating receptors NKp30 and NKp46 in NK cells isolated from the peripheral blood was associated with improved overall survival and a longer interval to development of castration resistance. Cytokine signaling in the TME, especially IL-6, was associated with resistance to NK cell-mediated cytotoxicity via modulating PD-L1 and NKG2D ligand levels in PCa cells. Peripheral blood NK cell dysfunction was also associated with poor clinical outcomes of PCa and higher disease stage. These findings suggest that modulation of NK cell activation could help overcome and challenge the limited benefit of T cell-centric strategies deployed by PD-1/PD-L1 or CTLA-4 inhibitors for treating mCRPC.

Novel strategies blocking the inhibitory receptor NKG2A expressed on NK and CD8+ cells are under clinical investigation to overcome resistance to anti-PD-1/PD-L1 checkpoint inhibition. NKG2A is an inhibitory checkpoint expressed on the cell surface of CD8, nearly half of circulating NK cells, and can be induced by cytokines such as IL-15 and IL-12. It is a heterodimer, bound with CD94 in humans and mice, and recognizes the non-classical class | major histocompatibility complex (MHC-I) molecules of human leukocyte antigen (HLA)-E. HLA-E expression is present in normal tissue as a protective “self” signaling, and it is up-regulated in various malignancies, including prostate cancer. The inhibitory function on NK cells is mediated through the binding of HLA-E to NKG2A/CD94, leading to the recruitment of the SHP-1 tyrosine phosphatase to the tyrosine-phosphorylated form of the intracytoplasmic tyrosine-based inhibitory motifs (ITIM). When phosphorylated, these motifs recruit phosphatases (SHP-1/2 or SHIP) responsible for transmitting inhibitory signals to immune effector cells. To our knowledge, we describe for the first time that HLA-E expression in prostate cancer cells is modulated by androgen signaling. AR inhibitors enzalutamide and darolutamide increased the expression of HLA-E in androgen-sensitive PCa cell lines. The ARi-driven upregulation of HLA-E could suppress NK cells and the innate immune response and contribute to the cold tumor microenviroment. Even though strategies enhancing NK cell cytotoxicity might display promising results, blocking the immune inhibitory HLA-E/NKG2A axis could result in additional benefits.

These results highlight the potential therapeutic implication of NK-activating strategies for the treatment of prostate cancer. The additive effect of androgen receptor inhibitors and monalizumab (anti-NKG2A) activating NK cells and enhancing PCa cell killing support further investigation of this combination in vivo with the potential for immediate clinical translation. Encouraging synergy of durvalumab and monalizumab in ongoing clinical trials for the treatment of lung cancer supports the clinical feasibility of this combination that could be expanded with the addition of androgen receptor inhibitors in advanced prostate cancer as a potential novel platform to modulate the cold TME in prostate cancer.

### Compositions

The present disclosure also provides pharmaceutical compositions that include effective amounts of the compounds, and the pharmaceutically acceptable salts thereof described above, and a pharmaceutically acceptable carrier. In certain embodiments, the disclosure also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

As used herein, “pharmaceutically acceptable salts” refers to an ionizable therapeutic agent that has been combined with a counter-ion to form a neutral complex. Lists of suitable salts are found, for example, in Remington's Pharmaceutical Sciences, 17th ed., Mack Publishing Company, Easton, Pa., 1985, p. 1418 and Journal of Pharmaceutical Science, 66, 2 (1977).

The terms “pharmaceutical” and “pharmaceutically acceptable” may refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable carrier” means a carrier that is useful for the preparation of a pharmaceutical composition that is: generally compatible with the other ingredients of the composition, not deleterious to the recipient, and neither biologically nor otherwise undesirable. “A pharmaceutically acceptable carrier” includes both one and more than one carrier. Embodiments include carriers for topical, ocular, parenteral, intravenous, intraperitoneal intramuscular, sublingual, nasal, and oral administration. “Pharmaceutically acceptable carrier” also includes agents for preparation of aqueous dispersions and sterile powders for injection or dispersions.

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

The term “treatment” may refer to the application of one or more specific procedures used for the amelioration of a disease. In certain embodiments, the specific procedure is the administration of one or more pharmaceutical agents. “Treatment” of an individual (e.g. a mammal, such as a human) or a cell is any type of intervention used in an attempt to alter the natural course of the individual or cell. Treatment includes, but is not limited to, administration of a therapeutic agent or a pharmaceutical composition, and may be performed either prophylactically or subsequent to the initiation of a pathologic event or contact with an etiologic agent. Treatment includes any desirable effect on the symptoms or pathology of a disease or condition, and may include, for example, minimal changes or improvements in one or more measurable markers of the disease or condition being treated. Also included are “prophylactic” treatments, which can be directed to reducing the rate of progression of the disease or condition being treated, delaying the onset of that disease or condition, or reducing the severity of its onset.

### Routes of Administration and Dosage Forms

The pharmaceutical compositions of the present disclosure include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein can be administered orally. Compositions of the present disclosure suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats, and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono-or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

Pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (e.g., glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

The pharmaceutical compositions of the present disclosure may be administered in the form of suppositories for rectal administration. These compositions can be prepared by mixing a compound of the present disclosure with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present disclosure is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

Examples of useful dermatological compositions which can be used to deliver the compounds and therapeutic agents to the skin are known in the art.

The compounds and therapeutic agents of the present disclosure may be incorporated into compositions for coating an implantable medical device, such as prostheses, artificial valves, vascular grafts, stents, or catheters. Suitable coatings and the general preparation of coated implantable devices are known in the art. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present disclosure provides an implantable drug release device impregnated with or containing a compound or a therapeutic agent, or a composition comprising a compound of the present disclosure or a therapeutic agent, such that said compound or therapeutic agent is released from said device and is therapeutically active.

### Dosages and Regimens

In the pharmaceutical compositions of the present disclosure, a compound is present in an effective amount (e.g., a therapeutically effective amount).

Effective doses/amounts may vary, depending on the diseases treated, the severity of the disease, the route of administration, the sex, age and general health condition of the subject, excipient usage, and the possibility of co-usage with other therapeutic treatments such as use of other agents and the judgment of the treating physician.

In some embodiments, an effective amount of the compounds, nucleic acids and the pharmaceutically acceptable salts thereof described above, can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0. 1 mg/kg to about 200 mg/kg; from about 0. 1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg).

In some embodiments, an effective amount of the compounds, nucleic acids and the pharmaceutically acceptable salts thereof described above is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

### Kits

In some embodiments, provided herein are packaged dosage forms, comprising a container holding a therapeutically effective amount of the compounds, nucleic acids and the pharmaceutically acceptable salts thereof described above, and instructions for using the dosage form in accordance with one or more of the methods provided herein.

The present dosage forms and associated materials can be finished as a commercial product by the usual steps performed in the present field, for example by appropriate sterilization and packaging steps. For example, the material can be treated by UV/vis irradiation (200-500 nm), for example using photo-initiators with different absorption wavelengths (for example, Irgacure 184, 2959), preferably water-soluble initiators (for example, Irgacure 2959). Such irradiation is usually performed for an irradiation time of 1-60 min, but longer irradiation times may be applied, depending on the specific method. The material according to the present disclosure can be finally sterile-wrapped so as to retain sterility until use and packaged (for example, by the addition of specific product information leaflets) into suitable containers (boxes, etc.).

According to further embodiments, the described dosage forms can also be provided in kit form combined with other components necessary for administration of the material to the patient. For example, disclosed kits, such as for use in the treatments described herein, can further comprise, for example, administration materials.

The kits may be designed in various forms based on the specific deficiencies they are designed to treat.

The dosage forms provided herein may be prepared and placed in a container for storage at ambient or elevated temperature. This is beneficial because transportation of commercially viable dosage forms may benefit from stability at temperatures greater than those requiring refrigeration or sub-freezing environments during transportation and storage at the site of use.

When the dosage forms provided herein are stored in a polyolefin plastic container as compared to, for example, a polyvinyl chloride plastic container, discoloration of the dosage form may be reduced. Without wishing to be bound by theory, the container may reduce exposure of the container's contents to electromagnetic radiation, whether visible light (for example, having a wavelength of about 380-780 nm) or ultraviolet (UV) light (for example, having a wavelength of about 190-320 nm (UV B light) or about 320-380 nm (UV A light)). Some containers also include the capacity to reduce adherence or adsorption of the active ingredient to the surface of the container, which could effectively dilute the concentration of active ingredient in the contained solution. Some containers also include the capacity to reduce exposure of the container's contents to infrared light, or a second component with such a capacity. Some containers further include the capacity to reduce the exposure of the container's contents to heat or humidity. The containers that may be used include those made from a polyolefin such as polyethylene, polypropylene, polyethylene terephthalate, polycarbonate, polymethylpentene, polybutene, or a combination thereof, especially polyethylene, polypropylene, or a combination thereof. In some embodiments, the container is a glass container. The container may further be disposed within a second container, for example, a paper container, cardboard container, paperboard container, metallic film container, or foil container, or a combination thereof, to further reduce exposure of the container's contents to UV, visible, or infrared light. Articles of manufacture benefiting from reduced discoloration, decomposition, or both during storage, include dosage forms that include compounds, nucleic acids and the pharmaceutically acceptable salts thereof. The dosage forms provided herein may need storage lasting up to, or longer than, three months; in some cases up to, or longer than one year. The containers may be in any form suitable to contain the contents—for example, a bag, a bottle, or a box.

### EXAMPLES

### Materials and Methods

### Cell Lines and Culture Conditions

Prostate cancer cell lines (obtained from ATCC) included PC3, DU145, LNCaP, and 22Rv1. Cells were grown in RPMI 1640 medium (Cytiva HyClone SH30027LS) supplemented with 10% fetal bovine serum, 1% sodium pyruvate, 1% GlutaMAX, and 1% penicillin/streptomycin at 37° C., 5% CO₂. Cells were trypsinized (0.25% trypsin) upon flask confluency and tested for Mycoplasma every 6 months. Even though 22Rv1 constitutively expresses the androgen receptor, it is resistant to androgen blockade due to a splice-site mutation (AR-V7) [14].

NK cell line NK-92 was obtained from ATCC (RRID: CVCL_2142). The cell line was grown in alpha-MEM medium supplemented with 10% FBS, 1% sodium pyruvate, 1% Glutamax, 1% penicillin/streptomycin, 0.1 mM of 2-mercaptoethanol, 12.5% of heat-inactivated horse serum, 1% Non-Essential Amino Acids (NEAA), 1% folic acid and 20 mM myoinositol at 37° C., 5% CO₂. NK-92 cells were kept at 4-5×10⁵ cells/ml seeding concentration and supplemented with 100 IU/ml of recombinant human IL-2 every two days. Cells were passaged using Ficoll PM400, sodium diatrizoate, and disodium calcium EDTA solution (Ficoll-Paque PLUS Cytiva 17-1440-02) every 10-14 days and up to 72 h before plating/experiments.

### Western Blot Analysis

Anti-apoptotic proteins and androgen receptor expression in prostate cancer cell lines and immune cells (NK-92) were examined using a western blot. A total of 1×10⁵ cells were plated and incubated in a 6 or 12-well plate (CELLTREAT 229111 and 229105) for 24 hours to allow the cells to adhere (tumor cells). Treatment conditions were added after cell adhesion or in conjunction with NK cells when these were analyzed. Cells were harvested and lysed using RIPA buffer containing protease inhibitor (Cell Signaling Technology 9806S) and phosphatase inhibitor (PHOSS-RO Roche 4906845001). Denaturing sample buffer was added, and samples were boiled at 95° C. for 10 minutes and an equal amount of protein lysate (10-20 ug) was electrophoresed through 4-12% SDS-PAGE gels (Invitrogen) and then transferred to PVDF membranes. The membrane was blocked with 5% milk in 1×TTBS and incubated overnight with the appropriate primary antibody (Cell Signaling c-IAP2 58C7 Rabbit mAb #3130; Cell Signaling XIAP Antibody #2042; Cell Signaling Mcl-1 (D2W9E) Rabbit mAb #94296; Cell Signaling Survivin (71G4B7) Rabbit mAb; Cell Signaling Ran Antibody #4462; Cell Signaling PARP 9542T mAb; Cell Signaling Bcl-2 Antibody #2876; Cell Signaling Bcl-xL (54H6) Rabbit mAb; Sigma Aldrich Monoclonal Anti-B-Actin antibody #A5441; Cell Signaling Androgen Receptor (E3S4N) Rabbit mAb (Carboxy-terminal Antigen) #70317). The primary antibody membranes were incubated in the appropriate HRP-conjugated secondary antibody (either mouse, Thermo Scientific #31430, or rabbit, Thermo Scientific #31460) for two hours. The levels of antibody binding were detected using ECL western blotting detection reagent and the Syngene imaging system.

### Establishing IC50 Doses

PCa cells were plated at a density of 5×10³ cells per well of a 96-well plate. Cells were treated with doses ranging from 0-250 μM of anti-androgens enzalutamide and darolutamide. For NK cell viability assays, 5×10³ cells were plated in a 96-well plate, and viability (72h) was measured using a CellTiterGlo assay. Bioluminescence imaging was measured using the Xenogen IVIS imager. IC50 doses were determined based on the dose-response curve collected from the data. Concentrations used in the experiments were correlated with those findings, matched with literature reports, and clinical correlation when possible.

### Co-Culture Assays

Prostate cancer cell lines were dyed with the blue CMAC live-cell dye (Thermo Fisher Scientific #C2110, per manufacturer protocol). Blue-fluorescent cancer cells were plated at a density of 10,000 cells per well of a 48-well plate. NK-92 cells were dyed with green CMFDA (Cayman Chemical Company #19583, per manufacturer protocol). Green-fluorescent NK-92 cells were added to the blue-fluorescent cancer cells at a 1:1 ratio. Enzalutamide (10 μM), darolutamide (20 μM), and 1 μM ethidium homodimer (EthD-1) were added at the same time as the NK cells. After 24 hours of co-culture, images of live cancer cells, live NK cells, and dead cells were taken using a fluorescent microscope. The co-culture experiments were run with internal control conditions and as a single treatment. Normalization was performed after analysis to take baseline death conditions into each experiment run. To optimize the high output of these experiments, a subset of co-cultures were performed using the ImageXpress® Micro Confocal High-Content Imaging. This analysis maintained the experiment conditions, drug concentrations, and the dyeing process. When GFP-labeled tumor cells were used for culture assays, they were added in a 1:1 ratio. Cell adhered over 24 h, and when patient-derived NK cells were added, a control well was trypsinized, and cells resuspended and counted to adjust a 1:1 ratio before treatment addition. The experiments with blocking antibodies were performed using the ImageXpress system. TRAIL blocking ab was purchased from Santa Cruz TRAIL Antibody (RIK-2): sc-56246, and IFN gamma blocking Monoclonal Antibody (NIB42, #6-7318-81) was purchased from Thermo Fisher. The blocking mAb was added when NK cells were combined with tumor cells.

### Statistical Analysis

Live cancer cells, NK cells, and dead cells were quantified using FIJI (Fiji Is Just ImageJ) software for images derived from fluorescent co-culture images. Images from the ImageXpress system were quantified using MetaXpress cytoplasmic/nuclear staining software. The percent of dead tumor cells in each well was quantified, and this percentage was normalized by subtracting baseline death from cancer cell-only wells, NK cell-only wells, or both. A two-way ANOVA was used to calculate the interaction effect between drug treatment and NK cells.

In groups with a significant interaction effect, data was further normalized in the cancer cell+NK cells+drug treatment well by subtracting out death in the cancer cell+drug well. A one-way ANOVA and multiple comparison analysis with subsequent t-tests were used to calculate the statistical significance of the difference between this group and the cancer cell+NK cell well. GraphPad Prism Version 9.5.1 was used for statistical analysis.

Generation of stably expressing GFP cell lines, Androgen Receptor, and Reporter inducible system.

### GFP Positive Cell Line Production

The prostate cancer cell lines (PC3, DU145, LNCaP, and 22Rv1) were seeded at 50% confluence in a 12-well tissue culture plate and adhered overnight. Then, they were transduced with lentivirus containing pLenti_CMV_GFP_Hygro [pLenti CMV GFP Hygro (656-4) was a gift from Eric Campeau & Paul Kaufman (Addgene viral prep #17446-LV)] at a multiplicity of infection of 10 for 48 hours before washing with PBS and replacing with fresh medium. The cells were then sorted for GFP-positivity using a BD FACSAria™ III Cell Sorter (RRID: SCR_016695).

### Plasmid Handling

Agar stabs with plasmid-containing bacteria were obtained from Addgene. Bacteria were streaked onto LB agar dishes with 100 μg/mL ampicillin and grown at 30° C. for 24 hours. Single colonies were then Midi-prepped (QIAGEN Plasmid Plus Midi Kit, 12943) according to the manufacturer's protocol. The plasmid sequence was verified by whole-plasmid sequencing (Plasmidsaurus) using Oxford Nanopore technology.

### Lentivirus Production

Five million HEK293T cells (ATCC CRL-3216) were seeded in a 10 cm tissue culture dish with 6 mL of antibiotic-free DMEM with 10% FBS (ATCC 30-2020) and adhered overnight. They were then transfected using 50 μL of Lipofectamine 2000 (Thermo Fisher 11668019), 10 μg of transfer plasmid [either pLENTI6.3/AR-GC-E2325 (Addgene 85128, pLENTI6.3/AR-GC-E2325 was a gift from Karl-Henning Kalland) or ARR3tk-eGFP/SV40-mCherry (Addgene 132360, ARR3tk-eGFP/SV40-mCherry was a gift from Charles Sawyers)], 5 μg of pMDLg/pRRE (Addgene 12251, pMDLg/pRRE was a gift from Didier Trono), 5 μg pRSV-Rev (Addgene 12253, pRSV-Rev was a gift from Didier Trono), and 2.5 μg of pMD2.G (Addgene 12259, pMD2.G was a gift from Didier Trono). After 16 hours, the medium was exchanged. Forty-eight hours after transfection, the medium was harvested, centrifuged at 500 g for 5 minutes, and the supernatant was sterile-filtered through a 0.45 μm polyethersulfone syringe filter (Millipore SLHPR33RS). The supernatant was then stored at −80° C. for future use.

### Lentivirus Transduction for pLENTI6.3/AR-GC-E2325 and ARR3tk-eGFP/SV40-mCherry

PC3 and DU145 cell lines were seeded at 50% confluence in a 12-well tissue culture plate and allowed to adhere overnight. Varying volumes of viral supernatant were added to each well for 48 hours. Wells were then washed with PBS and replaced with fresh medium. For pLENTI6.3/AR-GC-E2325, cells were selected with 2.5 μg/ml (PC3) and 5 μg/m (DU145) of blasticidin (InvivoGen ant-bl-1) for 7-9 days. For ARR3tk-eGFP/SV40-mCherry, cells were sorted for mCherry-positivity using a BD FACSAria™ III Cell Sorter (RRID: SCR_016695).

### Isolation of Human NK Cells

NK cells were isolated from patients harboring metastatic castration-sensitive prostate cancer using the MojoSort™ Human NK Cell Isolation Kit (#480053). Patients signed informed consent for IRB-approved research protocol number 2055-13, allowing the collection of peripheral blood samples. Cells were isolated from patients using a density-dependent methodology, Ficoll PM400, sodium diatrizoate, and disodium calcium EDTA solution (Ficoll-Paque PLUS Cytiva 17-1440-02). Following the suggested protocol from the manufacturer of negative selection sorting, non-natural killer cells were depleted by incubating the patient's whole PBMCs sample with the biotin antibody cocktail (BioLegend 480053) followed by incubation with magnetic Streptavidin Nanobeads. The magnetically labeled fraction (non-NK cell population) was retained using a magnetic separator (BioLegend 480019). The untouched NK cells (CD56₊, CD3₋) were collected, and the purity of isolation was determined by flow cytometry using anti-CD56 (BioLegend 362503). NK isolated cell populations were only kept in NK cell medium (see methods above) and further plated for experiments if the purity of the sample for CD56₊ was above 98%.

### Cytokine Analysis

A total of 3×10⁵ PC3, LNCaP, 22Rv1, and DU145 cells were plated per well of a 24-well plate and treated for 5-7 days with 15 nM of DHT (dihydrotestosterone) before ARi were aded. A total of 2×10⁵ of NK-92 were incubated in 15 nM of DHT for 72h before enza or darolutamide treatment. The medium was collected 24 hours after treatment and stored at −80° C. until readout. Samples were shipped to Brown University to run them in biological duplicate on a Luminex 200 Instrument (R&D LX200-XPON-RUO), which captures cytokines on magnetic antibody-coated beads and measures cytokine levels using a system based on the principles of flow cytometry (see https:/www.luminexcorp.com/luminex-100200/#overviewfor more information). A custom 52-52-cytokine panel was split into 34-plex and 18-plex assays (R&D LXSAHM) and was run on the Luminex 200 instrument according to the manufacturer's protocol.

### Flow Cytometry

PCa cells were plated at a density of 3×10⁵ cells per well of a 6-well plate and adhered overnight. In experiments where the treatment conditions included enza and darolutamide, cells were kept in culture in the presence of DHT (20-30 nM) for at least 72 h. After treatment, cells were trypsinized, stained, and incubated (Cell Staining Buffer SouthernBiotech #0225-01S) with the corresponding antibody for 45-60 min at 4° C. Viability dye was added with each run. NK-92 cells and patient-derived cells were stained similarly. Markers used for experiments included: Thermofisher HLA-E Monoclonal Antibody (3D12HLA-E), Alexa Fluor™ 488, eBioscience™, CD274 (PD-L1, B7-H1) Monoclonal Antibody (MIH1), eFluor™ 450, eBioscience™, Cell Signaling PD-1 (D4W2J) XPR Rabbit mAb, SYTOX™ Orange Dead Cell Stain, for flow cytometry, BioLegend APC anti-human CD159a (NKG2A) Antibody #375108, BioLegend APC anti-human CD69 Antibody #310909, BioLegend Alexa Fluor® 647 anti-human CD137 (4-1BB) [4B4-1] #309823. For staining of the patient-derived PBMCs and subsequent NK cell characterization, the following antibodies were used: BioLegend Anti-Perforin Mouse Monoclonal Antibody (PerCP Cy5.50, clone: dG9) #308113, BioLegend Anti-Granzyme B Mouse Monoclonal Antibody (FITC, clone: GB11) #515403, BioLegend Anti-CD56 Mouse Monoclonal Antibody (Alexa Fluor® 700, clone: 5.1H1) #362521, and BioLegend (PE anti-human CD45 Antibody) #304008.

### Example 1

This study aimed to investigate the effect of AR inhibitors (ARi) enzalutamide (Enza) and darolutamide (Daro) on the function of NK cells.

We conducted cell viability assays to investigate the effect of Enza and Daro on PC cells (LNCap, 22Rv1 [ARv7 mutation], DU145, PC3 [AR-]) and NK-92 cells. We performed co-culture experiments with PC and NK cells in a 1:1 ratio and analyzed immune cell-mediated tumor cell killing (ImageXpress Confocal HT). Cytokine profiling (Luminex 200) of culture supernatants was performed following treatment with ARi−/+ and anti-IFN-γ antibodies. In vivo studies were performed in NCr Nude mice harboring subcutaneous PC tumors treated orally with Daro (50 mg/kg/bid) and Enza (30 mg/kg/bid) for 4 weeks.

Treatment of co-cultures of PC with NK-92 cells with Ari significantly increased immune-mediated PC killing within 24 hours (control: 19%±2.7; Enza: 36%±3; Daro: 41%±4.2). Treatment of NK cells alone with ARi did not impact their viability. ARi increased the secretion of cytokines such as IL-2 (7.6 pg/ml+1.3 vs. 18 pg/ml±2.4, p=0.004), IL-1β (17.8 pg/ml±2.8 vs. 67 pg/ml±12.6, p=0.005), CXCL10 (224.2 pg/ml±16.6 vs. 504 pg/ml±42.4, p=0.017) and GDF-15 (member of the TGFβ superfamily; 8.4 ng/ml±0.43 vs. 14.3 ng/ml±3.2, p=0.042) and decreased VEGF (302.5 pg/ml±62.1 vs. 120 pg/ml±50.4, p=0.01), FGF-basic (7.4 pg/ml±1.6 vs. 3.6 pg/ml±2.1, p=0.007), M-CSF (70.9 pg/ml±14.2 vs. 25.3 pg/ml±4.5, p=0.01) and IL-8 (2645 pg/ml±201 vs. 1821 pg/ml±62.3, p=0.02). ARi increased NK-92 secretion of IFN-γ (15.2 pg/ml±5.4 and 48.2 pg/ml±8.2, p=0.01) and granzyme B (630 pg/ml±32.2 vs. 1024 pg/ml±102.4, p=0.001). The experiment suggests that ARi modulate NK cell function directly.

IFN-γ upregulation induced by ARi was blocked with IFN-γ mAb in the co-culture experiments and inhibited the NK cell-mediated killing of PCs. Analysis of PC tumors from NCr Nude Mice treated with Daro or Enza showed increased NK cell infiltration in the tumor microenvironment.

The results show that ARi promotes the activation of NK cells mediated by IFN-γ, leading to increased killing of PCs.

### Example 2

This study investigated the mechanisms of AR inhibitors (ARi) enzalutamide (Enza) and darolutamide (Daro) on the antitumor function of NK cells and potential enhancement with novel NK cell targeting checkpoint inhibitors.

We performed ATP-based (CellTiter-Glo) viability assays to investigate the effect of Enzalutamide and Darolutamide on PC cell lines (LNCap, 22Rv1 [ARv7 mutation], DU145, PC3 [AR-]) and NK-92 cells. We performed co-culture experiments with PC and NK cells at a 1:1 ratio throughout 4 h, 6 h, 12 h, 24 h, and 48 h periods. We analyzed immune cell-mediated tumor cell killing in the presence of Enza and Darolutamide (ImageXpress Confocal HT). Western Blotting was performed to evaluate different protein expression levels and their regulation related to sensitivity in mediating immune-mediated cell killing (apoptotic family). The immune-mediated killing of prostate cancer cell lines was further evaluated with cytokine profiling (Luminex 200) of culture supernatants. The signaling cytokines (without the presence of immune cells) produced and secreted by the prostate cancer cell lines and NK-92 cells alone were also analyzed following treatment with ARi−/+. We further investigated the immune-mediated killing of PC and NK cells by performing co-culture experiments with PC, NK, and T cells, in a 1:1 ratio, using IFN-γ blocking mAb (10 ug/ml) and RIK-2 (TRAIL mAb-10 ug/ml) in the presence of enzalutamide (Enza) and darolutamide (Daro). Immune cell-mediated tumor cell killing was analyzed (ImageXpress Confocal HT). The NK cell immune checkpoint inhibitor monalizumab (targeting NKG2A) was combined with ARi.

The effect of androgen pathway inhibitors in the expression of HLA-E, a negative and key negative modulator of NK cell function, was also performed using Flow Cytometry. PD-L1 expression levels were also measured. In advance, The NK cell immune checkpoint inhibitor monalizumab (targeting NKG2A and its ligand, HLA-E) was also combined with ARi.

In vivo studies were performed in NCr Nude mice harboring subcutaneous PC tumors treated orally with Daro (50 mg/kg/bid) and Enza (30 mg/kg/bid) for 4 weeks.

Preliminary Results and Basis for NK cell immune checkpoint blockade in conjunction with androgen pathway inhibitors:

Treatment of co-cultures of PC plus NK-92 cells with ARi significantly increased immune-mediated PC killing within 24 hours (control[C]: 20%±1.9; Enza: 40%±4.3; Daro: 36%±3.72, p<0.0001). Treatment of NK cells alone with ARi did not impact their viability suggesting a potential role for their use in adjunction with ARi. To further investigate this immune enhancement effect, different expression levels of pro-apoptotic and antiapoptotic proteins were evaluated on the PC cell lines as well as their specific cytokine secretion profile when treated with Enza and Darolutamide.

The DU145 and 22Rv1 cell lines expressed reduced levels of X-linked inhibitor of apoptosis (XIAP) compared to controls when treated with Enzalutamide (5, 10, and 15 uM) and Darolutamide (10, 20 uM) over 48 h. The DU145 cell line also expressed reduced levels of c-IAP2 when treated under the same conditions. The 22Rv1 also evidenced lower levels of BCL-XL when treated with APi. The AR-sensitive cell line LNCaP also expressed lower levels of XIAP and Survivin compared to controls when treated under the same conditions. These findings suggested a possible role of androgen pathway inhibitors in modulating anti-apoptotic proteins and rendering cells more susceptible to immune-mediated killing.

To investigate the direct role of IFN-γ in mediating this immune enhancement killing, IFN-γ upregulation induced by ARi was blocked with IFN-γ mAb (10 ug/ml) in the co-culture experiments, and the presence of the blocking IFN-γ mAb reversed the immune-mediated killing (near-complete). To achieve further suppression and completely inhibit the degree of immune enhancement killing induced by APi, treatment of PC with NK cells in the presence of Enza, IFN-γ mAb, and RIK-2 (TRAIL inhibitor) significantly reduced the NK cell-mediated killing of PCs (control[C]: 20%±1.9, [T]: 3.2%±2.6, p=0.003).

To evaluate a possible mechanism to further increase the degree of immune enhancement killing in the presence of API, co-cultures of PC cell lines and NK cells with Enzalutamide were performed also in the presence of the NKG2A blocking mAb, monalizumab. ARi immune enhancement effect was increased by NKG2A blockade with monalizumab ([C]: API immune enhancement: 31.2%±3.1, [T] monalizumab+API: 43.92%±4.52, p=0.0045). Flow cytometry analysis of the LNCaP cell line treated with Enza and Daro for 48 h, evidenced a decrease of HLA-E surface expression ([C]: 2.13%, [Enza]: 1.1% and [Daro]: 0.8%, p=0.005) as well as an increase PD-L1 expression. Treatment with Enza and Daro did not change significantly the surface levels of HLA-E and PD-L1 on the 22Rv1 cell line. These preliminary data suggest a possible role of the androgen pathway in the modulation of the ligand of the pivotal NK checkpoint regulator NKG2A, not previously described. Co-cultures of PC with NK-92 cells and NK cells derived from naive treated metastatic prostate cancer patients with ARi significantly increased immune-mediated PC killing effect within 24 hrs (control[C]: 20%±1.9; Enza: 40%±4.3; Daro: 36%±3.72, p<0.0001). This immune enhancement effect was abolished by IFN-γ mAb. The combination of TRAIL inhibitory mAb with Enza decreased the immune-mediated killing by 16±2.4% ([C]: 40%±4.3; Enza.+RIK-2: 25.6%±2.1, p=0.002).

The treatment of PC with NK cells in the presence of Enza, IFN-γ mAb, and RIK-2 significantly reduced the NK cell-mediated killing of PCs (control[C]: 20%±1.9, [T]: 3.2% ±2.6, p=0.003). ARi immune enhancement effect was increased by NKG2A blockade with monalizumab ([C]: 31.2%±3.1, [T]: 43.92%±4.52, p=0.0045).

The experiments show that ARi promotes NK cell killing of PC mainly by IFN-γ gamma and TRAIL mediated NK cell activation. ARi immune enhancement effect was increased by NKG2A blockade with anti-NKG2A monalizumab. ARi modulate the expression of NKG2A on NK cells and possibly influence the expression of their target, HLA-E in an AR dependent manner. ARi may modulate the expression of PD-L1 on NK cells.

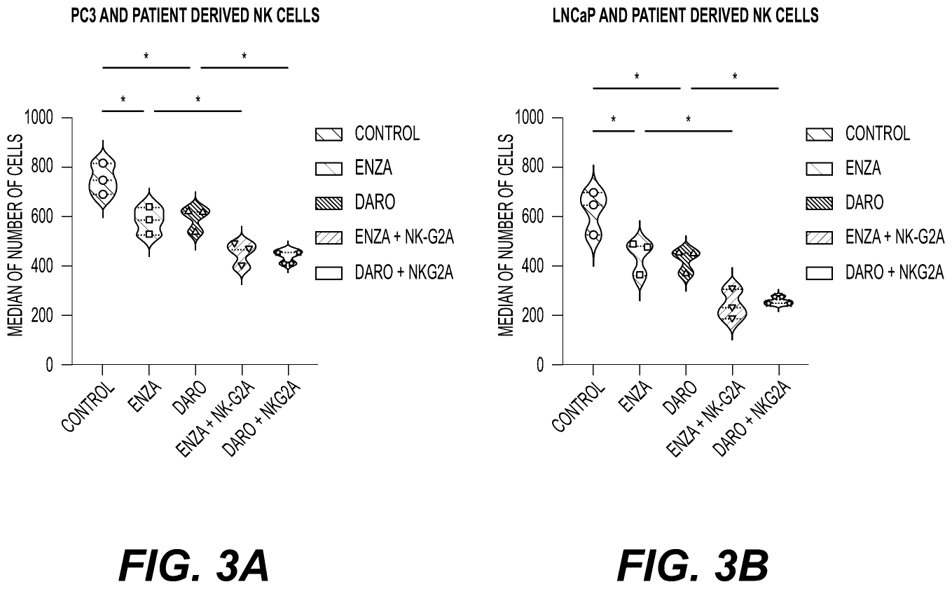

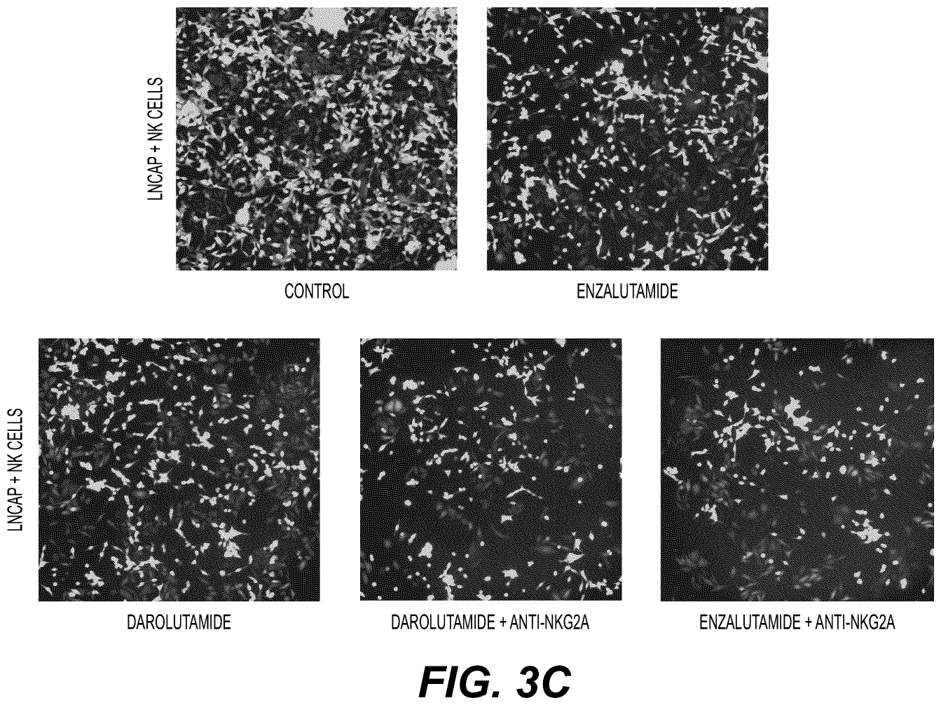

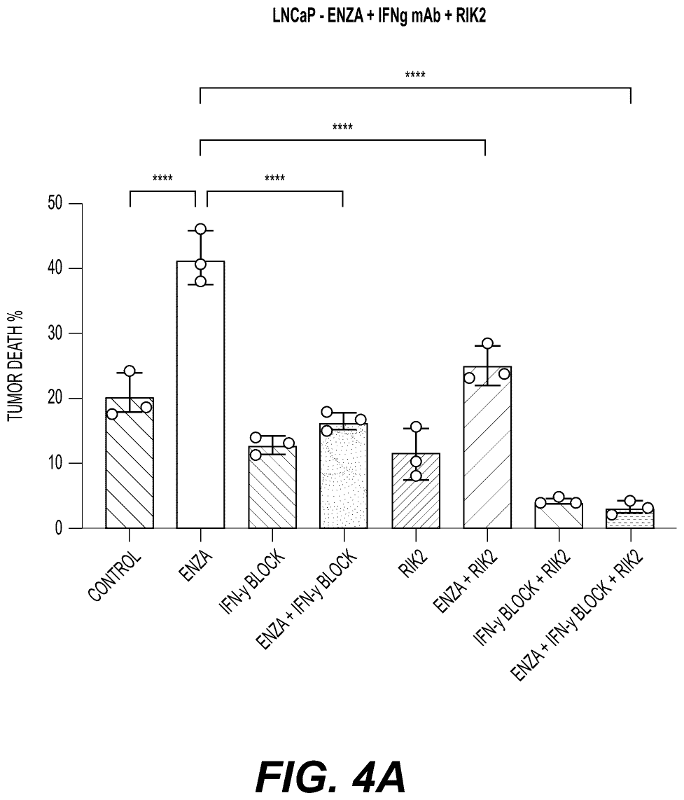

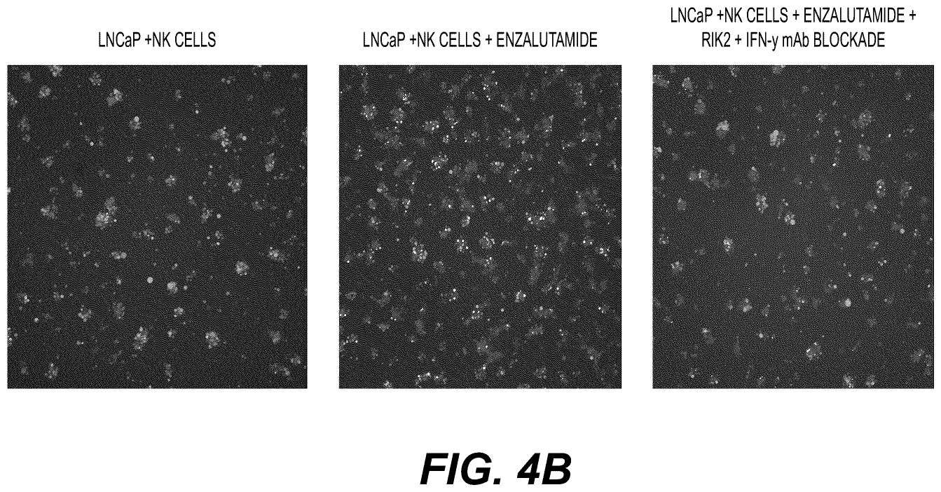

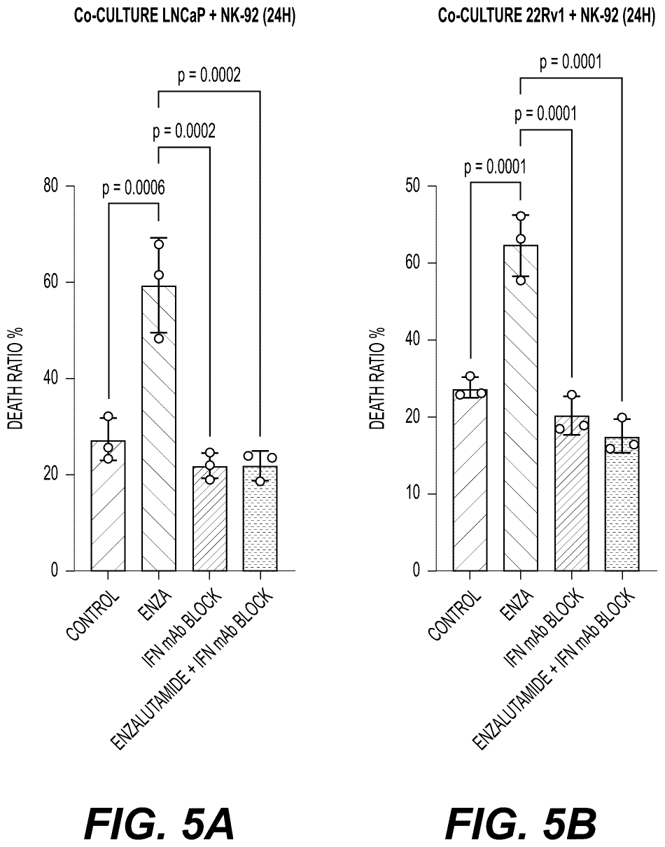

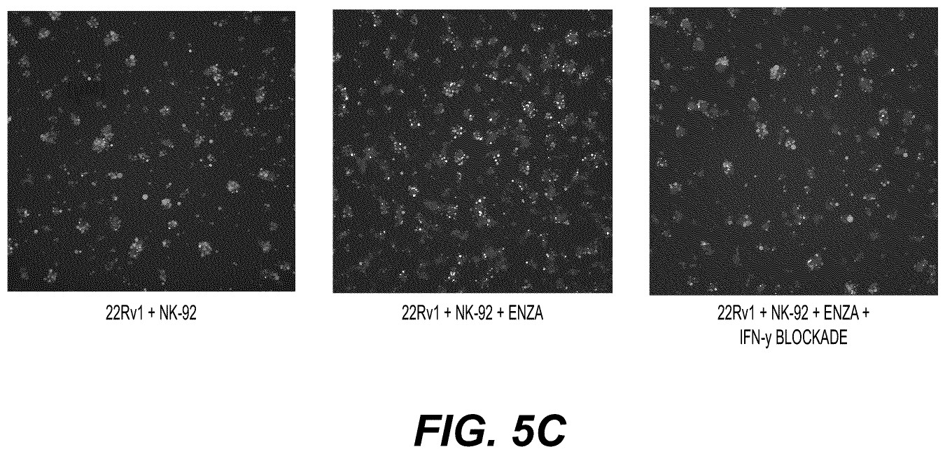

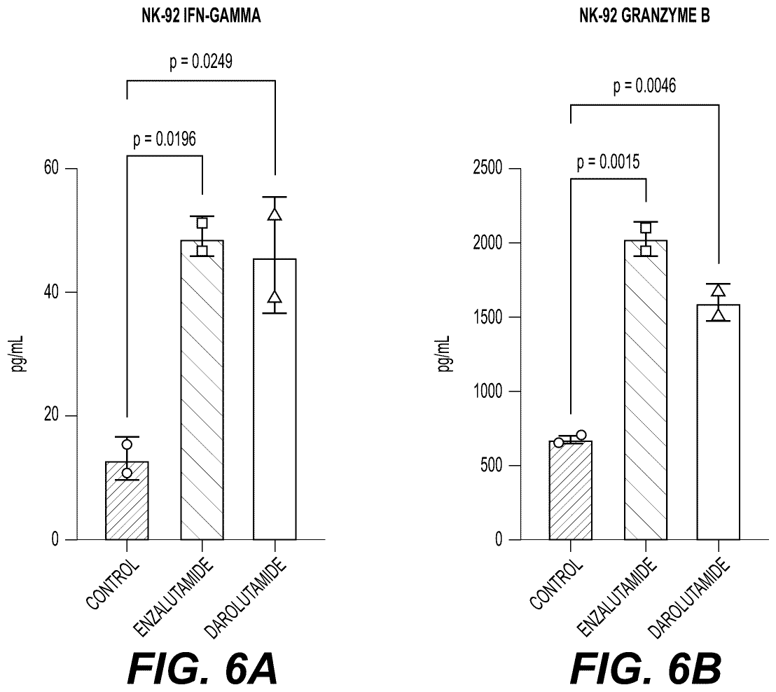

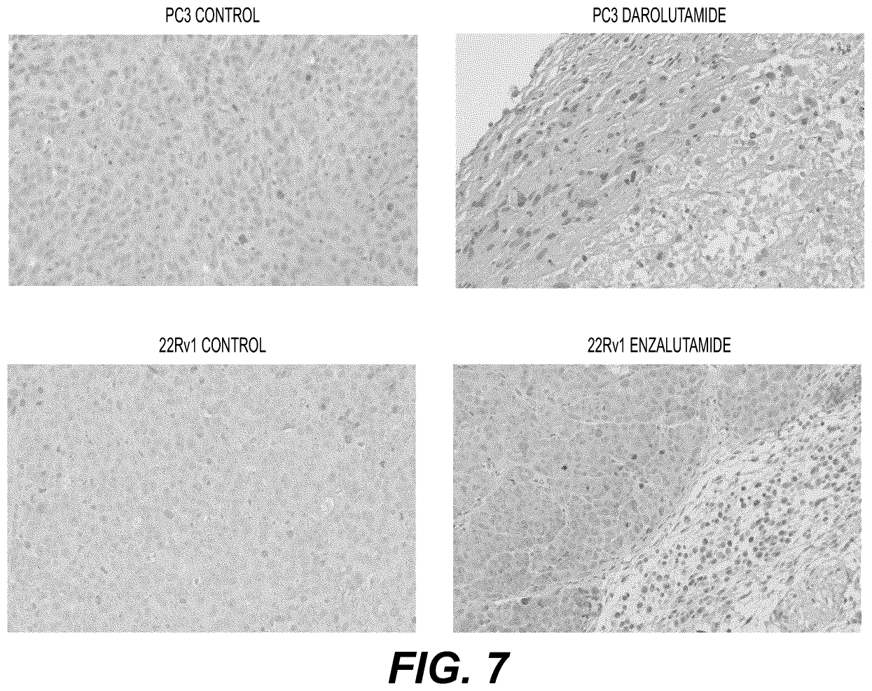

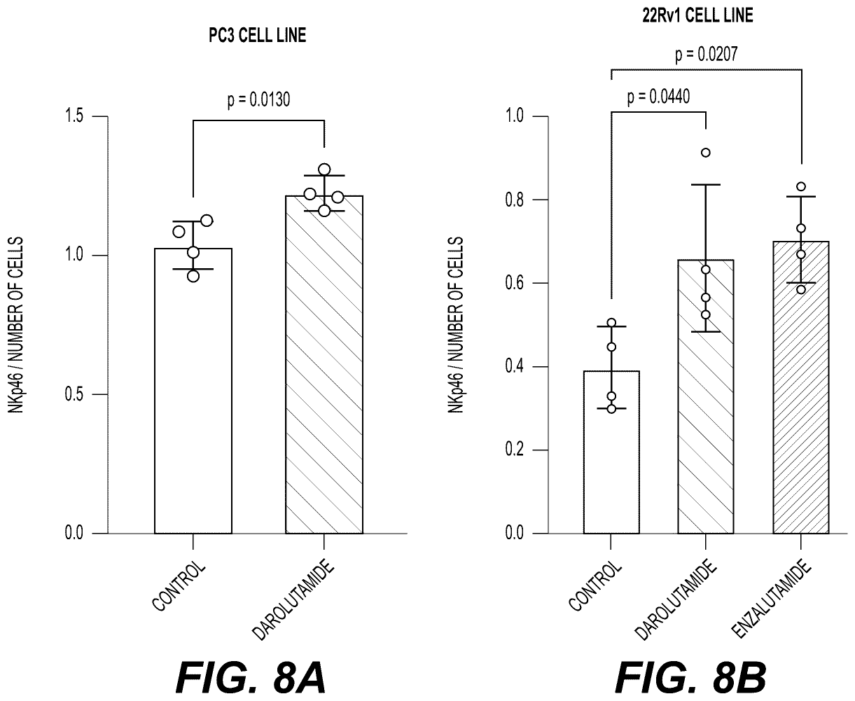

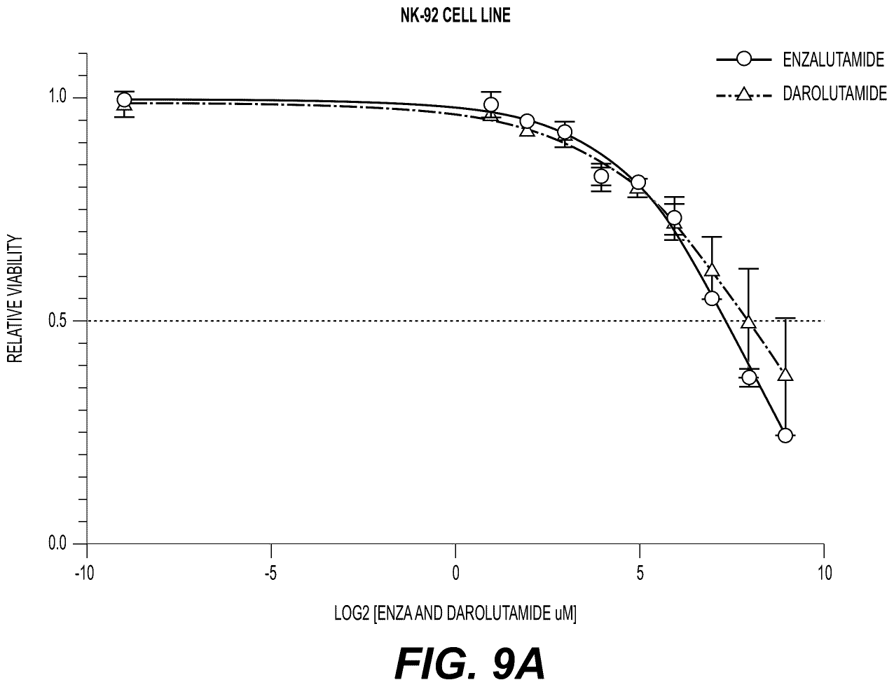

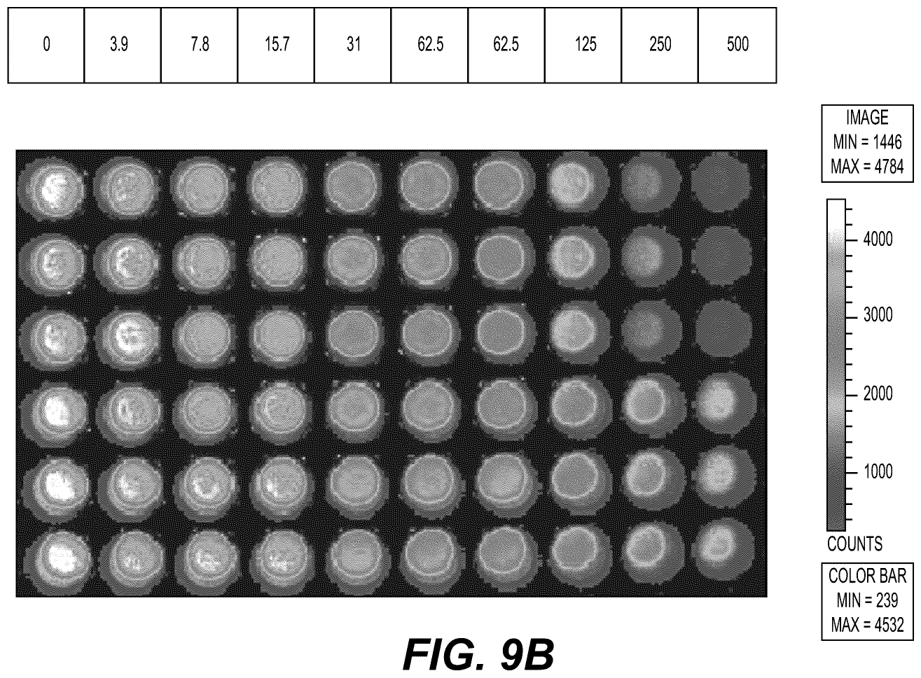

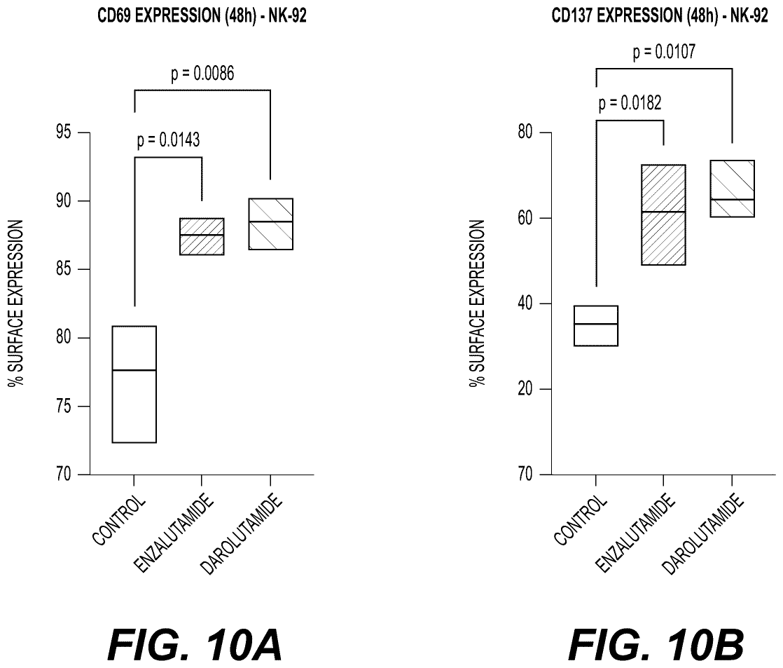

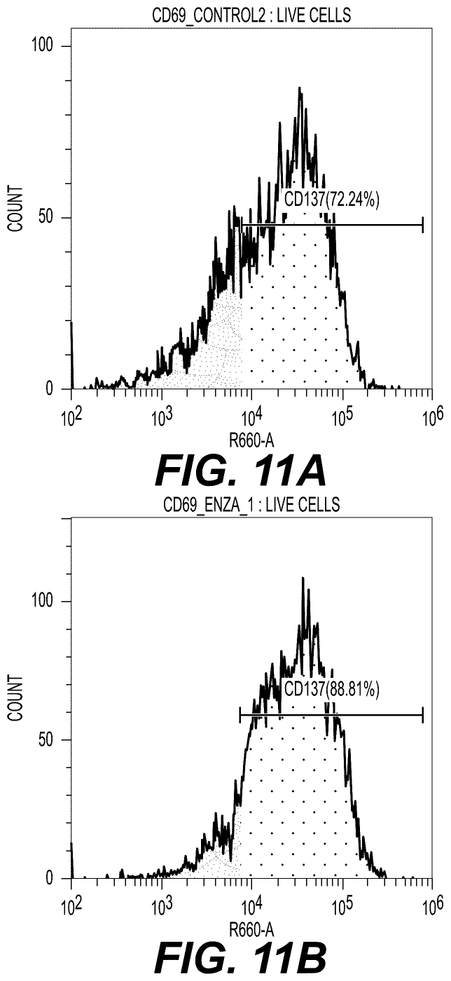

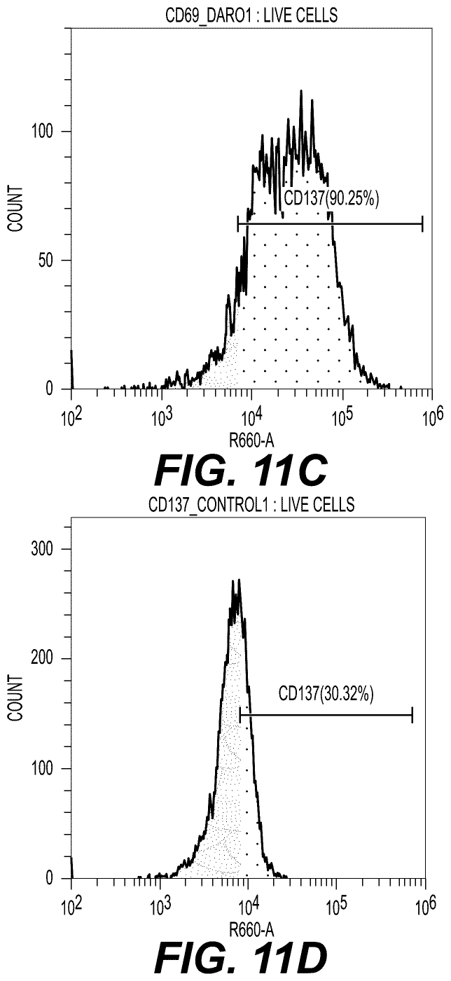

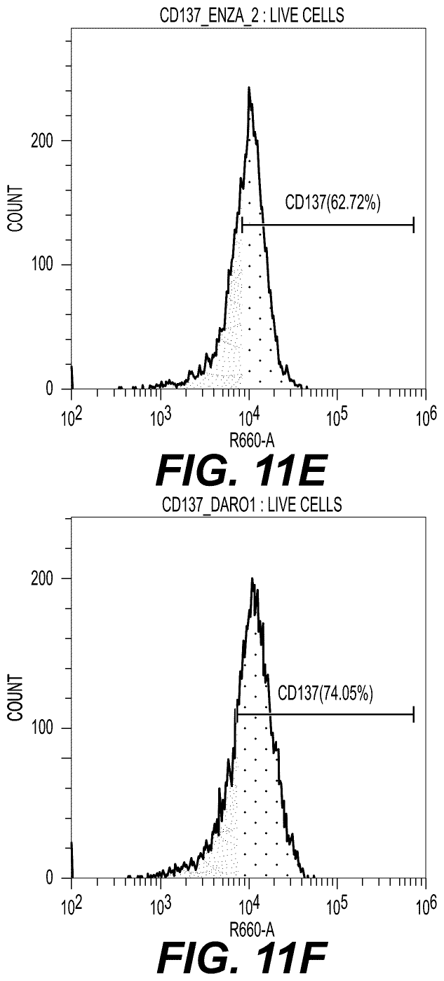

Adding anti-NKG2A mab monalizumab to darolutamide or enzalutamide therapy decreases median number of cells in a co-culture of PC3 and NK cells and LNCaP and NK cells as shown in FIG. 3A and FIG. 3B.

### Example 3

The blockade of the androgen receptor (AR) pathway is an effective treatment for prostate cancer (PCa), but many patients progress to metastatic castration-resistant prostate cancer (mCRPC). Therapies for mCRPC include AR inhibitors (ARi), chemotherapy, PARP inhibitors, and radioligands. Checkpoint inhibitor activity is limited to a small subset of MSI-H mCRPC. AR signaling modulates CD8+ T cell function, but its impact on NK cell (NKc) cytotoxicity is unknown. We investigated the effect of ARi on NKc activation, cytokine secretion, expression of inhibitory receptor NKG2A, and killing of PCa cells in vitro.

Methods: PCa cell lines (LNCaP, 22Rv1 [ARv7 mutation], DU145[AR-], PC3 [AR-]) were cocultured with NK-92 cells and treated with ARi (enzalutamide [enza] and darolutamide [daro]) or in combination with anti-NKG2A antibody monalizumab. Immune cell-mediated tumor cell killing assays and multiplexed cytokine profiling were performed. NKc expression of NKG2A and PCa cells expression of HLA-E were investigated by flow cytometry. The AR-negative cell lines (PC3 and DU145) were stably transduced with a functional AR pathway to evaluate the modulation of HLA-E by AR. The activation status of peripheral blood NKc isolated from patients with PCa before and post-initiation of androgen deprivation therapy (ADT) was investigated by flow cytometry.

Results: ARi significantly increased immune-mediated NK-92 cell killing of PCa cells independent of their sensitivity to androgen signaling. Cytokine analysis revealed that ARi-induced NKc activation is mediated by IFN-γ and TRAIL, as confirmed by blocking antibodies. ARi increased NKG2A expression in NK cells. Immune killing of PCa cells was enhanced with the combination of ARi and monalizumab. ARi also increased the expression of HLA-E, the ligand of the inhibitory NKG2A receptor, on PCa cell lines. Using AR-negative cell lines (PC3 and DU145) and stable transduction of AR, we demonstrate that androgen signaling regulates HLA-E expression. HDAC inhibitors (vorinostat and panobinostat) did not alter the androgen-induced expression of HLA-E in PCa cells. Mirroring the results from NK-92 cells, ADT also activated peripheral blood NK cells isolated from patients with metastatic PCa.

Thus ARi activates NK cells through upregulating IFN-γ and TRAIL and promotes the killing of PCa cells. This enhanced cytotoxic killing of PCa cells is augmented by monalizumab. ARi upregulates PCa cell's expression of HLA-E, suggesting a mechanism suppressing the innate immune response against PCa. These results support novel therapeutic strategies for PCa targeting NK activation with the combination of ARi and monalizumab.

Androgen receptor signaling blockade enhances NK cell-mediated killing of prostate cancer cells and sensitivity to NK cell checkpoint blockade. Nonetheless, ARi can potentially upregulate an NK cell inhibitor ligand (HLA-E), thus suppressing NK cell killing of PCa. This regulation is dependent on a functional AR signal on tumor cell lines. Adding an anti-NKG2a-HLA-E mAb with ARi further enhances the NK cell-mediated killing of PCa.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

Furthermore, references to patents and printed publications may have been made in this specification. Each of the above-cited references and printed publications are individually incorporated herein by reference in their entirety.

In closing, it is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

1. A method of treating prostate cancer in a patient in need thereof comprising administering a therapeutically effective amount of an androgen receptor inhibitor and an agent that activates natural killer (NK) cells to the patient.

2. The method of claim 1, wherein the androgen receptor inhibitor is selected from darolutamide or enzalutamide.

3. The method of claim 1, wherein the androgen receptor inhibitor is selected from flutamide, nilutamide, bicalutamide, apalutamide, proxalutamide, BMS-641988 or TRC-253.

4. The method of claim 1, wherein the agent that activates NK cells is selected from a natural killer cell receptor NKG2A inhibitor, or a tumor necrosis factor-related apoptosis-inducing ligand (TRAIL)-inducing compound.

5. The method of claim 4, wherein the NKG2A inhibitor is monalizumab.

6. The method of claim 4, wherein the TRAIL-inducing compound is a compound of formula

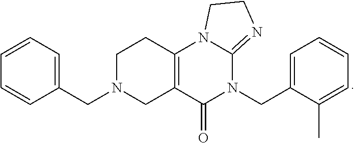 [SMILES: Cc1ccccc1CN1C(=O)C2=C(CCN(Cc3ccccc3)C2)N2CCN=C12]

7. The method of claim 1, comprising administration of one or more additional therapies selected from surgery, radiation therapy, poly (ADP-ribose) polymerase (PARP) inhibitors, checkpoint inhibitors, or chemotherapy.

8. The method of claim 1, wherein administering an androgen receptor inhibitor increases expression of non-classical human leukocyte antigen E (HLA-E) on NK cells.

9. The method of claim 1, wherein administering an androgen receptor inhibitor increases expression of programmed cell death ligand (PD-L1) on NK cells.

10. The method of claim 1, wherein administering an androgen receptor inhibitor increases secretion of interferon gamma (IFN-γ) from NK cells.

11. The method of claim 1, wherein administering an androgen receptor inhibitor increases secretion of granzyme B from NK cells.

12. The method of claim 1, wherein the prostate cancer is metastatic castration-resistant prostate cancer (mCRPC).

13. The method of claim 1, wherein the patient has been previously treated with androgen deprivation therapy (ADT).

14. A method for activating NK cells comprising contacting NK cells with an androgen receptor inhibitor.

15. The method of claim 14, wherein the androgen receptor inhibitor is selected from darolutamide or enzalutamide.

16. The method of claim 14, wherein the androgen receptor inhibitor is selected from flutamide, nilutamide, bicalutamide, apalutamide, proxalutamide, BMS-641988 or TRC-253.

17. A method for treating a cancer associated with androgen receptor signaling in a patient in need thereof comprising administering a therapeutically effective amount of an androgen receptor inhibitor and an agent that activates natural killer (NK) cells to the patient.

18. The method of claim 17, wherein the cancer is prostate cancer, breast cancer or melanoma.

19. A method for resensitizing treatment resistant prostate cancer cells, the method comprising modulating the expression of HLA-E in the prostate cancer cells.

20. The method of claim 19, wherein the expression of HLA-E in prostate cancer cells is modulated by contacting the cancer cells with an androgen receptor inhibitor and monalizumab.
